(19) United States
(12) Patent Application Publication
(10) Pub. No.: US 2024/0226372 A1
(43) Pub. Date: Jul. 11, 2024

(54) HYDROPHILIC POLY(THIOKETAL) URETHANE SCAFFOLDS AND METHODS OF USE THEREOF

(71) Applicant: Vanderbilt University, Nashville, TN (US)

(72) Inventors: Craig L. Duvall, Nashville, TN (US); Prarthana Patil, Nashville, TN (US); Mukesh K Gupta, Nashville, TN (US); Joshua Tyler McCune, Nashville, TN (US); Richard d'Arcy, Nashville, TN (US)

(21) Appl. No.: 18/288,034

(22) PCT Filed: Apr. 21, 2022
(86) PCT No.: PCT/US22/25856

§ 371 Date: Oct. 23, 2023

Related U.S. Application Data
(60) Provisional application No. 63/177,939, filed on Apr. 21, 2021.

(51) Int. Cl. A61L 27/18; A61L 27/56; A61L 27/60; C08G 18/52

(52) U.S. Cl. CPC A61L 27/18; A61L 27/56; A61L 27/60; C08G 18/52

(57) ABSTRACT

The presently-disclosed subject matter includes hydrophilic thioketal polymers and scaffolds formed therefrom.

### RELATED APPLICATIONS

This application claims priority from U.S. Provisional Application Ser. No. 63/177,939, filed Apr. 21, 2021, the entire disclosure of which is incorporated herein by this reference.

### GOVERNMENT INTEREST

This invention was made with government support under Grant No. R01 EB019409 awarded by the National Institutes of Health and Grant No. BMAT 1349604 awarded by the National Science Foundation. The government has certain rights in the invention.

### TECHNICAL FIELD

The presently-disclosed subject matter relates to poly(thioketal) urethane scaffolds. In particular, certain embodiments of the presently-disclosed subject matter relate to hydrophilic poly(thioketal) urethane and method of utilizing the same for wound treatment.

### BACKGROUND

Healing of wounds in healthy individuals occurs through four carefully orchestrated and partially overlapping phases of homeostasis, inflammation, proliferation, and remodeling. Under normal physiological environments, damage to epidermal barrier is efficiently restored whereas damage to deeper dermal injuries results in loss of functional tissue and excess scar formation. Various pre-existing morbidities result in the onset and persistence of chronic wounds, including venous and arterial blockages, diabetes, skin pressure and infection (FIG. 1).

In chronic wounds, healing is arrested in the inflammatory phase and is characterized by high levels of inflammatory cells, cytokines, reactive oxygen species (ROS) and bacterial infections. Additionally, upregulation of matrix metalloproteases (MMPS), local ischemia and wound hyperoxia further increase ECM degradation and oxidative stress pushing the wound into a prolonged cycle of inflammation resulting in impaired wound healing. These physiological aberration lead to failure of granulation tissue formation, wound contraction, and poor epithelialization of the wound. Potential strategies for the treatments for chronic wounds have been targeted towards sequestering MMPs and ROS, modifying the ECM to be more robust and stable to create an environment that is conducive to cell growth and migration (FIG. 1).

Another strategy for treatment of chronic wounds includes acellular dermal substitutes (FIGS. 2A-C). Bioresorbable (natural and synthetic) skin substitutes are currently marketed for the treatment of full thickness wounds. Most clinically used dermal substitutes are sourced from naturally occurring materials such bovine or porcine extracellular matrix (ECM) such as Integra Bilayer Wound Matrix. These materials are susceptible to enzymatic degradation through MMPs and rapid loss in mechanical properties resulting in wound contraction, scar formation and loss of native tissue function. ECM based dermal substitutes have been associated with high costs of production and can't be used to treat patients with bovine/porcine allergies.

More recently, synthetic polymers have been used as an alternative source to engineer skin substitutes. Synthetic polymers offer high tunability, reproducibility and control over molecular composition and architecture making them a favorable class of biomaterials for skin generation applications. Polymers such as poly (lactic acid) (PLA), poly (lactic-co-glycolic acid) (PLGA), and poly (caprolactone) (PCL), comprise a class of synthetic polymers known as polyesters. Polyesters are hydrolytically sensitive and undergo degradation into non-toxic components that are then cleared from the body. It is difficult to optimize the degradation properties of polyesters because they are not cell-responsive and because they are susceptible to autocatalytic degradation due to build-up of acidic degradation byproducts. This can lead to formulation of products that either rapidly lose mechanical integrity resulting in wound contraction, or that persist too long in the wound and cause a foreign body response, chronic inflammation, and fibrosis.

Poly(thioketal)-urethane (PTK-UR) has also been explored as a dermal substitute that can be used as a temporary closure for wounds and to provide mechanical scaffolding for the new granulation tissue. Existing PTK-URs have utilized mostly dithiol-based monomers and formed the thioketal (TK) bond during the condensation polymerization process. However, there are limitations to the generation of materials using dithiol monomers having more than two ethylene glycol (EG2) groups (e.g., poor polymerization control/yield, cycle formation vs forming linear chains). This limits the current PTK-UR materials to relatively hydrophobic formulations

Accordingly, there remains a need for effective wound treatment materials and strategies, in particular more hydrophilic scaffolds that provide a improved immune and wound healing response.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments, the presently-disclosed subject matter is directed to a hydrophilic thioketal polymer comprising a structure according to Formula I:

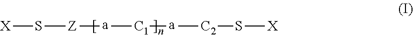

wherein a includes a thioketal; C₁ includes a zwitterionic monomer; C₂ includes poly(ethylene glycol) (PEG); x includes OH, NH₂, SH, hydroxyalkyl, aminoalkyl, or thioalkyl; Z includes —C₁—, —C₂—, or —C₁-a-C₂—; n is between 0 and 15; m is between 0 and 15; and if one of either n or m is 0 the other is at least 1. In some embodiments, the thioketal includes the following structure:

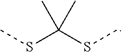

In some embodiments, the zwitterionic monomer includes:

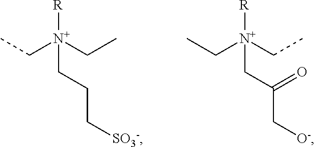

multiples thereof, or combinations thereof. In some embodiments, the PEG includes

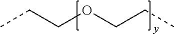

and y is between 0 and 2. In some embodiments, X includes a hydroxyalkyl. In some embodiments, the hydroxyalkyl includes a hydroxy(C₁-C₆)alkyl. In some embodiments, the hydroxyalkyl includes hydroxyethyl.

In some embodiments, n is 0. In some embodiments, Z is —C₂—. In some embodiments, the polymer includes:

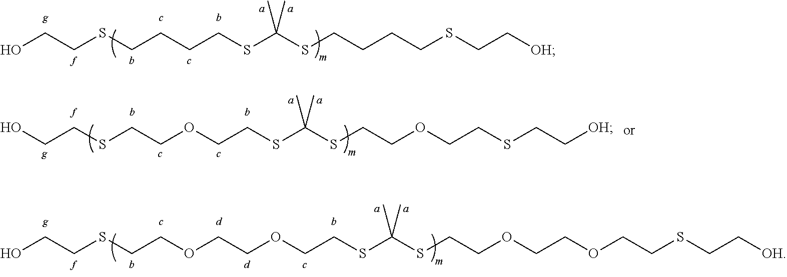

In some embodiments, both n and m are greater than 0. In some embodiments, Z is —C₂—. In some embodiments, the polymer includes:

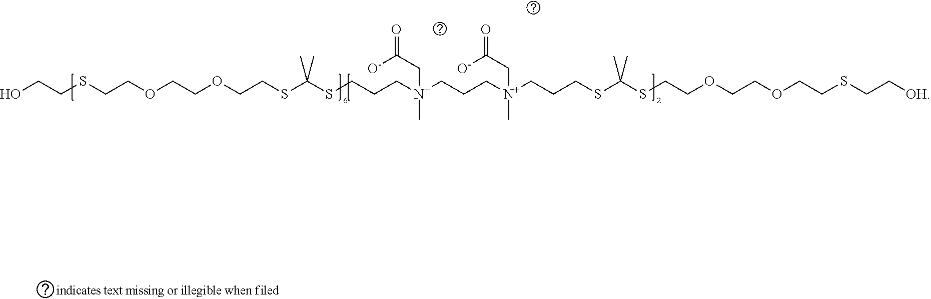

In some embodiments, Z is —C₁-a-C₂—. In some embodiments, the polymer includes:

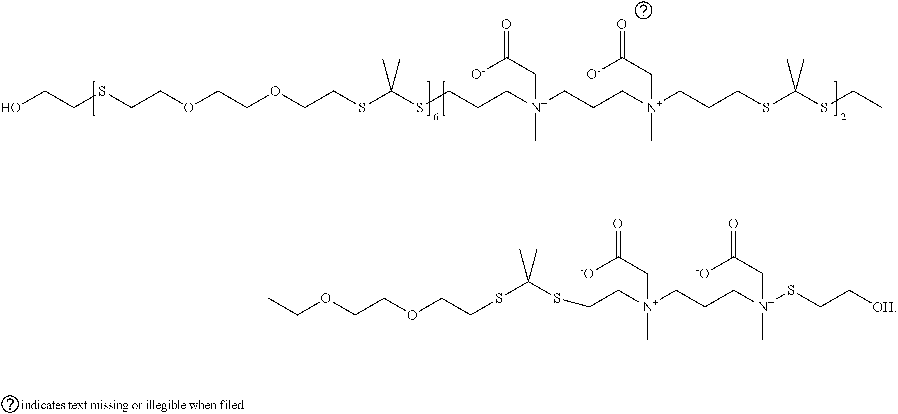

Also provided herein, in some embodiments, is a hydrophilic thioketal polymer including a structure according to Formula II:

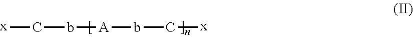

wherein A includes a thioketal crosslinker; b includes carbamate or carbonate; C includes poly(ethylene glycol) (PEG); x includes OH, NH₂, SH, hydroxyalkyl, aminoalkyl, or thioalkyl; and n is between 1 and 10. In some embodiments, the PEG includes

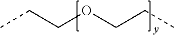

and y is between 3 and 15. In some embodiments, y is at least 7.

Further provided herein, in some embodiments, is a hydrophilic thioketal polymer including a structure according to Formula III:

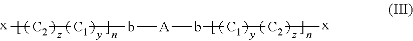

wherein A includes a thioketal crosslinker; b includes a 2° or 3° amide; C₁ includes a poly(peptide); C₂ includes a poly(peptoid); x includes OH, NH₂, SH, hydroxyalkyl, aminoalkyl, or thioalkyl; y is from about 0 to about 1; z is from about 1 to about 0; and n is between 1 and 15. In some embodiments, the polymer includes:

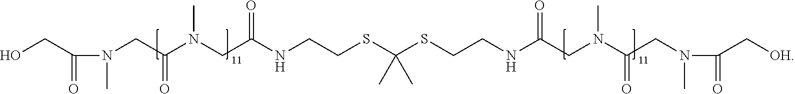

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description refers to the following drawings, wherein:

FIG. 1 shows a schematic illustrating proper and impaired wound healing.

FIGS. 2A-C show examples of prior attempts to address impaired wound healing.

FIG. 3 shows a schematic illustrating that reactive liquid molding of isocyanates and poly(thioketal) polyols results in covalently crosslinked 3D network with interconnected pores that facilitate tissue infiltration.

FIGS. 4A-B show schematics illustrating polyol synthesis. (A) Homobifunctional poly(thioketal) diol synthesis. (B) Thioketal diamine crosslinker and EG7 TK polyol synthesis.

FIGS. 5A-C show graphs and images illustrating PTK diol characterization. (A) Characterization of PTK diols using ¹H NMR. (B) Verification of terminal hydroxyl groups on the PTK through ATR-FTIR. (C) Quantification of terminal hydroxyls using ¹⁹F NMR.

FIGS. 6A-G show graphs and images illustrating increased EG content in PTK polyols. (A) Water contact angle measure of urethane films fabricated with polyester and poly(thioketal) polyols (Mean±SD, n=3). (B) Compressive Young's modulus measurement of hydrated PU scaffolds (Mean±SD, N=6 (2 scaffolds from 3 different batches). (C) Swell ratio measured 24 post incubation of PU scaffolds in PBS. (D) Cell Viability of NIH 3T3s measured 24 hours post sequential treatment with 50 μM H₂O₂ and polyol solution (matched TK content) (Mean±SD, n=4). (E) DPPH assay shows inhibition of free radicals by PTK-UR scaffolds treated with 200 μM of DPPH ethanol solution. (F) Dose dependent degradation of PTK-UR scaffolds measure in PBS, 0.2%, 2% and 20% H₂O₂ with 0.001, 0.01 and 0.1M CoCl₂. (G) SEM images showing loss of pore structure of PTK-UR scaffolds under oxidative conditions.

FIGS. 7A-G show graphs and images illustrating EG7 PTK-UR scaffolds facilitate tissue integration and neovascularization 10 days post implantation in porcine full thickness wounds. (A) Representative trichrome images (low mag and high mag) showing bulk integration of scaffolds and tissue infiltration into PU scaffold pores. (B) Quantification of tissue infiltration measured as % area occupied by granulation tissue (Mean±SD, n=3/4 wounds). (C) Quantification of bulk scaffold integration measured based on wound thickness (mm) occupied by scaffold (Mean±SD, n=3/4 wounds). (D) Representative images of neo-vasculature (VWF) within polyurethane scaffolds using. (E) Quantification of blood vessel area (positive brown pixels) within scaffold infiltrating granulation tissue. (F) Representative images showing CCR7+ macrophage infiltration into polyurethane scaffolds. (G) Clustergram analysis of inflammation-related gene expression in PU treated porcine wounds at POD 10. Gene expression is plotted as fold change relative to PE treatment.

FIGS. 8A-C show images and graphs illustrating increased rate of wound closure and re-epithelialization of porcine wounds treated with EG7 PTK-UR help restore barrier function of epidermal layer. (A) Representative photographic images of porcine wounds treated with polyurethane scaffolds comparing wound size and appearance up to 24 days post implantation. (B) Representative images showing CytoK14 positive keratinocytes covering newly formed granulation tissue post PU scaffold treatment. (C) Wound closure measured 24 days post wounding (Mean±SD, n=3-8 wounds) (top) and quantification of CytoK14 positive keratinocytes at the epithelial wound edge as a measure of re-epithelialization (Mean±SD, n=3-8 wounds) (bottom).

FIGS. 9A-B show images and graphs illustrating decreased inflammatory response associated with EG7 PTK-UR treated skin wounds promote functional tissue restoration. (A) Representative images showing CCR7, Arg-1 and CD3 positive immune cells associated with polyurethane material in granulation tissue 24 days post implantation. (B) Clustergram of inflammation-related gene expression in porcine wounds 24 days post PU treatment.

FIGS. 10A-E show images and graphs illustrating that EG7 PTK-UR scaffolds allow robust tissue regeneration and repair compared to more hydrophobic PTK-UR variants in vivo through increased ECM deposition and remodeling. (A) Representative trichrome images of neogranulation tissue 24 days post scaffold implantation. (B) Wound score compiled through blinded histomorphometric analysis of trichrome and H&E images of scaffold treated wounds. (C) Histological analysis of inflammation, granulation tissue, epithelialization and collagen deposition scored on a scale of 0-5. (D) Differential expression of ECM-related genes in PU scaffold treated wounds (Heat map is plotted as fold regulation relative to PE treatment). (E) qPCR analysis of fold change of ECM components and remodeling related genes normalized to PE treatment (Mean±SD, n=6-8 wounds).

FIGS. 11A-C show images and graphs illustrating that EG7 PTK-UR treated wounds show higher blood perfusion with similar rates of wound closure compared to clinically approved dermal substitutes. (A) Representative photographic images of porcine wounds treated with Integra BWM, Restrata, Novosorb and EG7 PTK-UR comparing wound size and appearance up to 31 days post implantation. (B) Quantification of wound area. (C) LDPI measurement of blood perfusion (normalized to unwounded skin).

FIGS. 12A-D show images and graphs illustrating effective immune modulation and ECM remodeling in PTK-UR treated wounds result in increased quality of wound repair compared to clinically approved polyester-based dermal matrix. (A) Representative histological images of wounds treated with PTK and PE based urethane foams. (B) Wound healing scores for PTK-UR and Novosorb treated wounds. (C) Fold regulation of inflammatory genes. (D) Heat map showing fold regulation of ECM-related genes.

FIGS. 13A-K show images and graphs illustrating that increasing EG content within PTK diols increases PU scaffold hydrophilicity and radical reactivity. (A) Schematic of controlled variation of the number (0-7) of ethylene glycol (EG) units in the PTK diol backbone. (B) Reactive liquid molding of isocyanates and poly(thioketal) diols yields covalently crosslinked three-dimensional polyurethane networks with interconnected pores (scanning electron microscopy (SEM) image) that permit cell infiltration. (C-D) Urethane (UR) film contact angle measurements (C) and correlation between measured contact angles and computed poly(thioketal) (PTK) diol Log P values (D) (ChemAxon). (E) UR scaffold modulus measured under compression in hydrated, aqueous conditions. (F) Radical scavenging capacity of PTK-UR compared to PE-UR scaffolds measured through DPPH inhibition. (G) Cytoprotective properties of PTK diols (matched thioketal content) determined after cell (NIH 3T3 fibroblasts) exposure to 25 μM or 50 μM 50 μM of H₂O₂ for 24 hours. (H) Ex vivo H₂O₂ concentration measured within polyester-urethane (PE-UR) and EG7 PTK-UR scaffolds explanted from the subcutaneous space in mice using Amplex Red. (I) Scaffold mass loss over 30 days in PBS, 0.2% H₂O₂ in 0.001 M CoCl₂, 2% H₂O₂ in 0.01 M CoCl₂, or 20% H₂O₂ in 0.1 M CoCl₂ at 37° C. (J) Degradation constants derived from the experimental data. (K) SEM images of PTK-UR scaffolds incubated in PBS or 2% H₂O₂/0.01 M CoCl₂ at day 0 and day 15; scale bar=200 μm. Data is presented as Mean±SD, n=3-4 scaffolds. ns, not significant. *P<0.05, **P<0.01, ***P<0.001, ****P <0.0001 by analysis of variance (ANOVA).

FIGS. 14A-H show images and graphs illustrating pig skin wound 10 days post implantation: hydrophilic EG7 PTK-UR chemistry shows better wound bed integration and neovascularization and a less pro-inflammatory wound microenvironment. (A) Representative trichrome images showing bulk integration of scaffold within the wound bed (low magnification, scale bar=1000 μm) and cellular infiltration and extracellular matrix (ECM) deposition within scaffold pores (high magnification, scale bar=250 μm). vWF immunohistochemistry (IHC) shows vascularization (brown) within the scaffold-infiltrating granulation tissue (scale bar=250 μm). Asterisks indicate scaffold remnants. (B) Quantification of bulk scaffold integration measured as wound thickness occupied by scaffolds. (C-D) Correlation between PTK diol ethylene glycol (EG) content and percent tissue infiltration within the scaffold pores (C) and PTK diol EG content and blood vessel area (D). (E) Representative MPO (neutrophils) and CD206 (M2 macrophages) IHC images of scaffold infiltrating tissue (scale bar=100 μm). (F-G) Quantified staining at day 10 for (F) MPO⁺ neutrophils and (G) CD206⁺ macrophages. (H) Bulk tissue gene expression analysis of inflammatory mediators in porcine skin wound bed 10 days post scaffold implantation. Color-coded heat map showing row normalized z-scores (n=4 wounds per group) of inflammatory marker gene expression.). Data presented as Mean±SD, n=3-4 wounds, ANOVA * **** P<0.05, ** P<0.01, *** P<0.001, ****P<0.0001.

FIGS. 15A-D show images and graphs illustrating that EG7 PTK-UR shows complete re-epithelization and overall enhanced wound healing than more hydrophobic scaffolds in day 24 pig skin wounds. (A) Representative images showing scaffold implantation and wound resolution over a period of 24 days. (B) Representative cytokeratin14 immunohistochemistry (IHC) showing wound re-epithelialization. (C) Wound closure measurements. Top: Quantified wound area (day 24) relative to initial wound size (day 0). Bottom: Percentage of wound length covered by cytokeratin14⁺(CytK14⁺) keratinocytes. (D) Representative trichrome images of granulation tissue analyzed 24 days post scaffold implantation (high mag, scale bar=2 mm and low mag, scale bar=250 μm). Wound score was assessed through treatment blinded histopathologist analysis of trichrome and H&E images for granulation tissue, collagen deposition, epithelialization, and inflammation, yielding a cumulative wound healing score for each scaffold type tested. Data presented as Mean±SD, n=6-10 wounds, * P<0.05, ** P<0.01, *** P<0.001, by analysis of variance (ANOVA).

FIGS. 16A-G show images and graphs illustrating that EG7 PTK-UR scaffolds elicit a less pro-inflammatory immune phenotype relative to more hydrophobic variants in day 24 pig skin wounds and day 21 mouse subcutaneous implants. (A) CCR7, CD3, and αSMA IHC of pig excisional skin wounds 24 days post treatment, scale bar=100 μm. (B) Quantification of CCR7 staining intensity as a function of distance from scaffold remnant edges. (C) Quantification of CD3⁺ pixel area within the wound. (D) qRT-PCR quantification of expression of pro-inflammatory genes TNFA, IFNG, CSF2, and CD40LG within pig wound scaffolds at day 24. (E) Scaffold-infiltrating myeloid and lymphoid populations quantified 21 days post subcutaneous implantation of EG2 PTK (empty bars) and EG7 PTK (pink filled bars) scaffolds in mice. (F) Percentage of FOXP3+ and GATA3+ infiltrating CD4 helper T cells in EG2 vs EG7 scaffolds. (G) Expression of CD301b in EG7 scaffold infiltrating CD206+ macrophages compared to EG2 PTK scaffolds. Data presented as Mean±SD, n=6-10 wounds, * P<0.05, ** P<0.01, *** P<0.001, **** P<0.0001 by analysis of variance (ANOVA).

FIGS. 17A-B show an image and graphs illustrating that EG7 PTK-UR scaffolds promote higher expression of pro-growth, ECM, and remodeling genes relative to more hydrophobic scaffolds in day 24 porcine wounds. (A) Bulk tissue differential gene expression of genes encoding ECM components, remodeling enzymes, and growth factors 24 days post-wound. Gene expression was analyzed and displayed as color-coded heat map showing log₂(FC) relative to PE 900t control. (B) Expression fold change (FC) of selected genes relative to 900t polyester (PE) treated wounds. Data presented as Mean±SD, n=4-10 wounds, * P<0.05, ** P<0.01, *** P<0.001, by analysis of variance (ANOVA).

FIGS. 18A-E show images and graphs illustrating that EG7 PTK-UR scaffold wound healing benchmarking versus Integra BWM and NovoSorb BTM in day 31 pig excisional wounds. (A) Images showing scaffold implantation and temporal closure of 2×1 cm porcine skin wounds treated with collagen-based Integra Bilayer Wound Matrix (BWM), polyester based NovoSorb Biodegradable Temporizing Matrix (BTM), or EG7 poly(thioketal)-urethane (PTK-UR) foams. (B) Kinetics of wound closure; arrows indicate timepoint of manufacturer-recommended removal of protective layer from Integra and NovoSorb. (C) Relative blood perfusion within scaffold-bearing wounds measured by laser doppler perfusion imaging (LDPI). (D) Trichrome and H&E images of wound sections 31 days post treatment with different dermal substitutes showing quality of infiltrating tissue and residual cell response to scaffold remnants (high mag, scale bar=2 mm and low mag, scale bar=250 μm). (E) Semi-quantitative analysis of wound healing through treatment-blinded pathohistological scoring of trichrome and H&E images for collagen deposition, epithelialization, and foreign body giant cells (FBGC), yielding a cumulative wound healing score. Data presented as Mean±SEM (B, C) and Mean±SD (E), n=6-10 wounds, * P<0.05, ** P<0.01, *** P<0.001, **** P<0.0001, by analysis of variance (ANOVA).

FIGS. 19A-D show images and graphs illustrating that EG7 PTK-UR scaffolds induce more vascularization and higher expression of wound healing-related and anti-inflammatory genes in day 31 pig wounds compared to NovoSorb. (A) vWF, CCR7, and CD3 immunohistochemistry (IHC) images of NovoSorb and EG7 PTK-UR treated wounds; scale bar =250 μm. (B) Quantification of vWF, CD3, and CCR7 positive pixels using ImageJ. (C) Relative expression of genes encoding extracellular matrix (ECM) components, remodeling enzymes, and growth factors related to wound healing and remodeling. (D) EG7 poly(thioketal)-urethane (PTK-UR)-treated wound expression of pro-inflammatory (black) and anti-inflammatory genes (green) (relative to NovoSorb). Data presented as Mean±SD, n=6-10 wounds, * P<0.05, *** P<0.001, by analysis of variance (ANOVA).

FIGS. 20A-K show images and graphs illustrating that EG7 PTK-UR scaffolds promote wound closure and re-epithelialization of porcine ischemic wounds at day 17. (A) Schematic of raised bipedicle ischemic flap model with excisional wounds. (B) Images over time of ischemic flap excisional skin wounds treated with NovoSorb or EG7 poly(thioketal)-urethane (PTK-UR). (C) Kinetics of ischemic wound surface area change after treatment with polyurethane foams; arrow indicates timepoint of removal of NovoSorb protective upper layer. (D) Blood perfusion quantified as total flux within scaffold implanted ischemic wound beds; arrow indicates timepoint of removal of protective layer from NovoSorb, a procedure that disrupts granulation tissue and stimulates a neovascularization response. (E-F) Cytokeratin14 (CytK14) immunohistochemistry (IHC) visualization (E) and quantification (F) of end point percent wound re-epithelization. (G) Representative microscopic images of wound sections stained with H&E and trichrome, showing the extent of cellular infiltration (pink/red in both images) and deposition of collagen (green in trichrome stain) within infiltrated voids of polyurethane foams. (H-I) Wound healing sub-category scores (H) and cumulative wound healing index (I) quantified by histopathological categories. (J-K)Immunohistochemistry (IHC) showing spatial organization and density of (J) MPO+ neutrophils and (K) CCR7+ macrophages. Data represented as Mean±SD, n=4 wounds, * P<0.05, ** P<0.01, by analysis of variance (ANOVA) for C and D and t-test for F, H, and I. Arrows in B, C, and D indicate the removal of the top layer of NovoSorb.

FIG. 21 shows a schematic and images illustrating the synthesis and H NMR characterization of EG0, EG1 and EG2 PTK diols. Synthesis scheme for condensation polymerization of BDT (EG0), MEE (EG1) and EDDT (EG2) dithiol monomers to obtain PTK dithiol polymers and their functionalization with bromoethanol to synthesize PTK diol polymer. On the right, ¹HNMR spectra of BDT PTK diol, MEE PTK diol and EDDT PTK diol showing accurate chemical composition.

FIG. 22 shows a schematic and images illustrating the synthesis and H NMR characterization of EG7 PTK diol. ROS-degradable thioketal diamine crosslinker and EG7 PTK diol through the reaction of TK diamine and amine reactive OH-PEG-NPC/NPC-PEG-NPC. On the right, ¹H-NMR spectroscopy of compounds 1, 2, 3, 5 and 6 are shown.

FIGS. 23A-C show graphs and a table illustrating characterization of PTK diols. (A) GPC elutograms for EG0, EG1, EG2, and EG7 polymers. (B) FTIR spectra of hydroxyl terminated PTK polymers with hydroxyl absorbance peak seen at 3400 cm-1 (black arrow). (C) Table showing number average molecular weight (M), dispersity index, thioketal repeating unit (RU), hydroxyl number (OH #, calculated through ¹⁹F NMR), and glass transition temperate (T_{g}) of EG0, EG1, EG2, and EG7 PTK diols.

FIGS. 24A-B show a graph and a table illustrating swell ratios and physical properties of PE and PTK-based urethane foams. (A) Swell ratio of PE and PTK based urethane foams hydrated in PBS. (B) Physical properties of PE-UR and PTK-UR scaffolds. Data is presented as Mean±SD. ***P<0.001, ****P<0.0001 by analysis of variance (ANOVA).

FIG. 25 shows representative SEM images of in vitro PTK and PE-UR scaffold degradation incubated in hydrolytic media (PBS) for 15 days. Scale bar=200 μm.

FIG. 26 shows representative SEM images of in vitro PTK and PE-UR scaffold degradation incubated in oxidative media simulated by 2% H₂O₂ 0.01M CoCl₂ for 15 days. Scale bar=200 μm.

FIG. 27 shows graphs illustrating in vitro degradation kinetics of PTK-UR scaffolds showing dose dependent degradation in oxidative media and relative stability in hydrolytic media over 30 days. Dashed lines represent MATLAB model-generated curves for degradation kinetics.

FIGS. 28A-B show graphs illustrating quantification of (A) tissue infiltration and (B) blood vessel area within scaffold infiltrating tissue 10 days post-wounding of porcine skin. Data presented as Mean±SD, n=3-4 wounds.

FIGS. 29A-B show images and a graph illustrating 8-OHdG IHC and DNA oxidation products of PTK-UR and PE-UR. (A) 8-OHdG IHC of PE-UR and EG7 PTK-UR treated day 10 porcine wounds. (B) DNA oxidation products in wounds treated with PTK-UR scaffolds compared to PE-UR. Mean±SD, n=9 high powered fields acquired from 2-3 wounds per scaffold type. Data presented as Mean±SD. P<0.05 by Welch's t-test.

FIGS. 30A-B show a graph and an image illustrating wound area and tissue infiltration of scaffold-treated porcine skin wounds. (A) Quantification of porcine skin wound area remaining open over a period of 24 days of scaffold-treated porcine skin wounds. (B) Representative trichrome image showing tissue infiltration within EG0 PTK-UR scaffold pores 20 days post implantation. Scaffolds were extruded from the wound post application. Data presented as Mean±SD (n=3-4 wounds per group).

FIGS. 31A-B show images and tables illustrating gating strategies and antibody clones. (A) Gating strategies for myeloid and lymphoid populations. Cells were gated on SSC and FSC, followed by doublet discrimination to isolate the single cell population based of FSC-A and FSC-H. Cells were gated on live immune cells (DAPI+CD45+) or (Viability dye+CD45+) prior to phenotypic specific gating. (B) Tables of antibody clones used in flow cytometry and immunophenotyping of myeloid and lymphoid cells

FIGS. 32A-B show images and graphs illustrating tissue infiltration, immune cell recruitment, and change in leukocyte sub-populations following EG2 and EG7 PTK-UR implantation. (A) 6 mm×1.5 mm EG2 and EG7 PTK-UR scaffolds implanted in subcutaneous pockets of C57BL/6 mice and explanted after 21 days. Representative H&E images showing tissue infiltration and immune cell recruitment into scaffold pores (high mag, scale bar=1 mm and low mag, scale bar=250 μm). (B) Change in leukocyte sub-populations in EG2 PTK (gray) and EG7 PTK (pink) cellular infiltrates measured 7, 14, and 21-days post implantation. Data presented as Mean±SD, n=6 mice, * P<0.05, ** P<0.01, *** P<0.001, **** P<0.0001 by analysis of variance (ANOVA).

FIGS. 33A-B show images illustrating treatment of wounds with various scaffolds. (A) Images of porcine skin wounds treated with Integra BWM, NovoSorb BTM or EG7 PTK-UR scaffolds showing remaining wound area and scaffold resolution. Arrows indicating removal of Integra BWM protective silicone layer on day 14 and BTM polyurethane layer on day 21. (B) Representative LDPI flux images showing blood perfusion within the wound. Arrows indicate removal of protective top layer of Integra and NovoSorb-treated wounds. n=6-10 wounds.

FIG. 34 shows a graph illustrating in vivo degradation rate of EG7 PTK-UR scaffolds in porcine excisional skin wounds. Data presented as Mean±SD, n=4-10 wounds, ** P<0.01, **** P<0.0001 by analysis of variance (ANOVA).

FIG. 35 shows images illustrating Arginase-1 IHC of NovoSorb- and EG7-PTK-UR-treated wounds. Scale bar=250 μm.

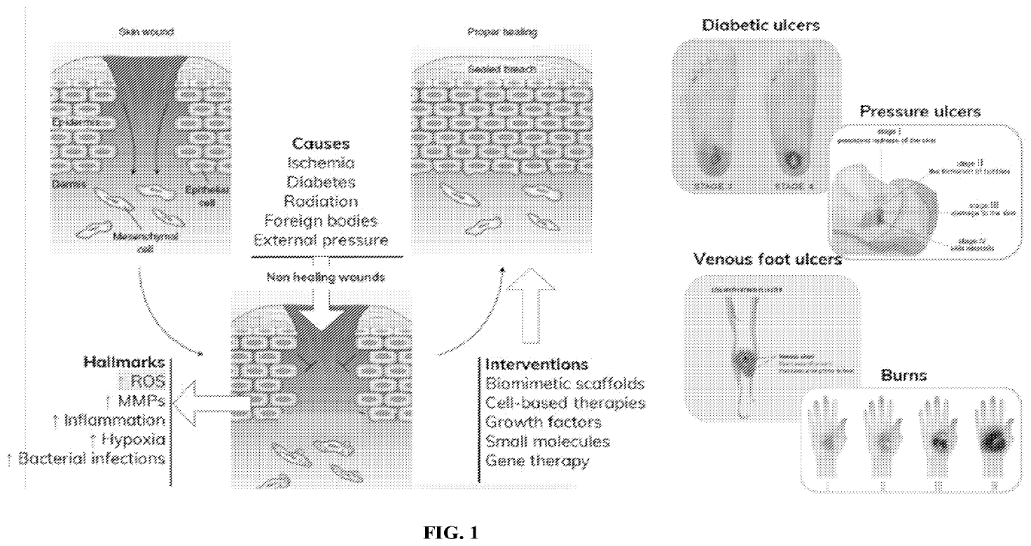

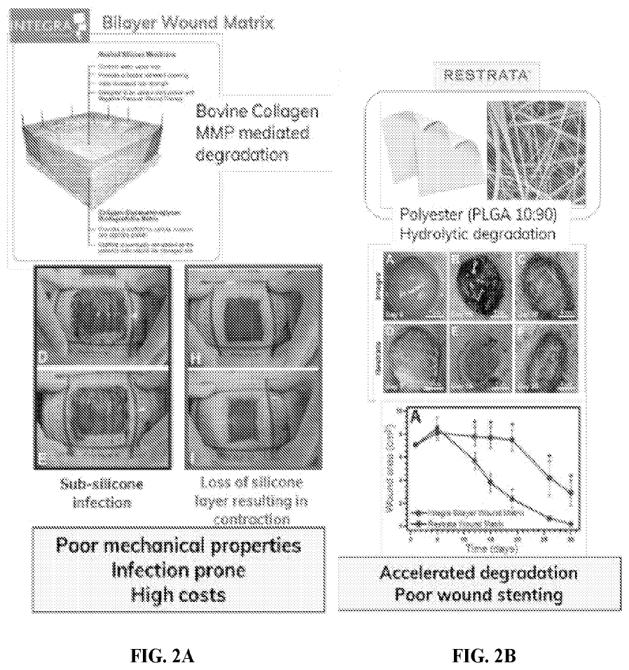

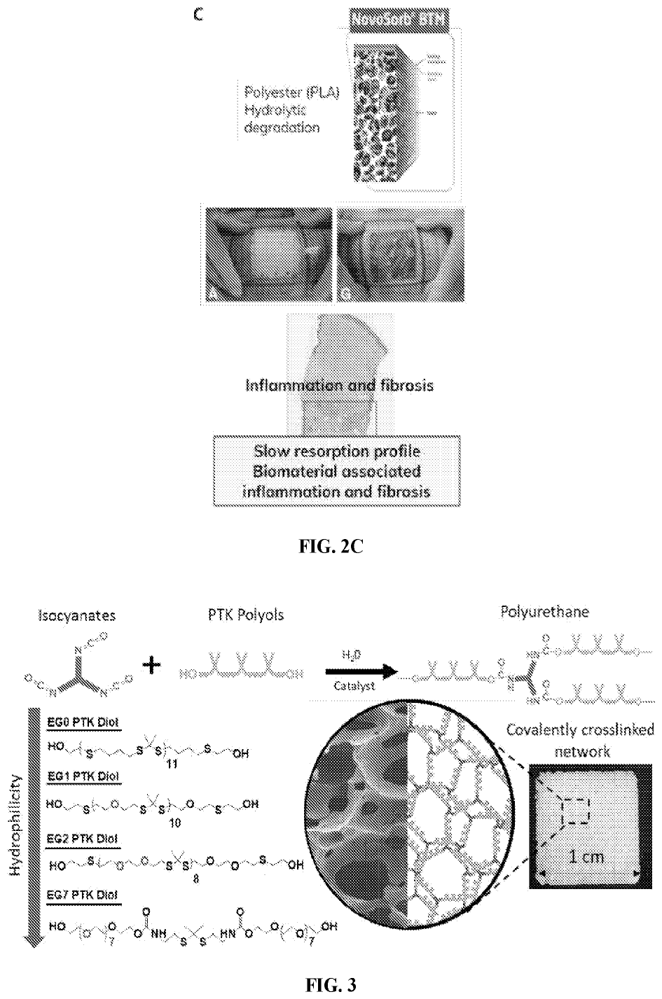

### DEFINITIONS

The term “bioactive agent” or “biologically active agent” is used herein to refer to compounds or entities that alter, promote, speed, prolong, inhibit, activate, or otherwise affect biological or chemical events in a subject (e.g., a human). For example, bioactive agents may include, but are not limited to osteogenic, osteoinductive, and osteoconductive agents, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug. In certain embodiments, the bioactive agent is a small molecule.

Antimicrobials used as bioactive agents in embodiments of the present invention may be selected from one that does little to no harm to the healing process. Clinically, antibiotics may be selected for their spectrum or ease of administration to the patient. When selecting an antibiotic for local delivery, the physical characteristics (charge and hydrophobicity) and state (liquid or powder) of the drug may also be considered. Additionally, the antimicrobials' effects on eukaryotic cells may be considered when developing an embodiment of the present invention, including a dual-delivery scaffold embodiment. In vitro studies that evaluated the effect of eight concentrations (ranging from 0 to 5,000 mg/ml) of 21 antibiotics on the viability and activity of osteoblasts found that vancomycin, a tricyclic glycopeptide antibiotic that is efficacious for treating infections caused by gram-positive bacteria such as Staph. aureus, may have the least detrimental effects on osteoblast function. All other antibiotics in the study reduced the alkaline phosphatase (ALP) activity at doses that were 10-50 times lower than that of vancomycin. Other studies also indicate that vancomycin has less adverse effects on osteoblasts than other commonly used antibiotics in vitro. Furthermore, vancomycin may not impede bone growth in fractures in vivo. Some embodiments comprise an antibiotic selected from the group consisting of clindamycin, cefazolin, oxacillin, rifampin, trimethoprim/sulfamethoxazole, vancomycin, ceftazadime, ciprofloxacin, colistin, and imipenem. Some embodiments comprise of silver ions or silver nanoparticles of different sizes.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formulary-20, published by the United States Pharmcopeial Convention, Inc., Rockville MD, 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21 C.F.R. §§ 330.5, 331 through 361, and 440 through 460, and drugs for veterinary use listed by the FDA under 21 C.F.R. §§ 500 through 589, all of which are incorporated herein by reference, are also considered acceptable for use in accordance with the present invention.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes. Some degradation may occur due to the present of reactive oxygen species.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “biomolecules” as used herein, refers to classes of molecules (e.g., proteins, amino acids, peptides, polynucleotides, nucleotides, carbohydrates, sugars, lipids, nucleoproteins, glycoproteins, lipoproteins, steroids, natural products, etc.) that are commonly found or produced in cells, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active fragments or subunits. In some embodiments, the biomolecule is a growth factor, chemotactic factor, cytokine, extracellular matrix molecule, or a fragment or derivative thereof, for example, a cell attachment sequence such as a peptide containing the sequence, RGD.

The term “carbohydrate” as used herein, refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

The term “composite” as used herein, is used to refer to a unified combination of two or more distinct materials. The composite may be homogeneous or heterogeneous. For example, a composite may be a combination of bone particles and a polymer; a combination of bone particles, polymers and antibiotics; or a combination of two different polymers. In certain embodiments, the composite has a particular orientation.

The term “contacting” refers to any method of providing or delivering a scaffold on to or near tissue to be treated. Such methods are described throughout this document, and include injection of a biodegradable scaffold on to a tissue wound and/or molding a biodegradable scaffold in a mold and then placing the molded scaffold on a tissue wound. In some embodiments contacting refers to completely covering a skin wound, and optionally the surrounding skin, with a biodegradable polyurethane scaffold. In some embodiments contacting refers to placing a biodegradable polyurethane scaffold between two or more bone fragments that have fractured. In various aspects, a scaffold can be contact an existing tissue wound, and in further various aspects a polyurethane scaffold can be contacted prophylactically; that is, to prevent a wound from forming on tissue.

The term “flowable polymer material” as used herein, refers to a flowable composition including one or more of monomers, pre-polymers, oligomers, low molecular weight polymers, uncross-linked polymers, partially cross-linked polymers, partially polymerized polymers, polymers, or combinations thereof that have been rendered formable. One skilled in the art will recognize that a flowable polymer material need not be a polymer but may be polymerizable. In some embodiments, flowable polymer materials include polymers that have been heated past their glass transition or melting point. Alternatively or in addition, a flowable polymer material may include partially polymerized polymer, telechelic polymer, or prepolymer. A pre-polymer is a low molecular weight oligomer typically produced through step growth polymerization. The pre-polymer is formed with an excess of one of the components to produce molecules that are all terminated with the same group. For example, a diol and an excess of a diisocyanate may be polymerized to produce isocyanate terminated prepolymer that may be combined with a diol to form a polyurethane. Alternatively or in addition, a flowable polymer material may be a polymer material/solvent mixture that sets when the solvent is removed.

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The terms “polynucleotide”, “nucleic acid”, or “oligonucleotide” as used herein, refer to a polymer of nucleotides. The terms “polynucleotide”, “nucleic acid”, and “oligonucleotide”, may be used interchangeably. Typically, a polynucleotide comprises at least three nucleotides. DNAs and RNAs are exemplary polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as RNAi, siRNA, or shRNA.

The terms “polypeptide”, “peptide”, or “protein” as used herein, include a string of at least three amino acids linked together by peptide bonds. The terms “polypeptide”, “peptide”, and “protein”, may be used interchangeably. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide” as used herein, refer to any polymer or oligomer of carbohydrate residues. Polymers or oligomers may consist of anywhere from two to hundreds to thousands of sugar units or more. “Oligosaccharide” generally refers to a relatively low molecular weight polymer, while “polysaccharide” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., reduced, oxidized, phosphorylated, cross-linked). Carbohydrate polymers or oligomers may include natural sugars (e.g., glucose, fructose, galactose, mannose, arabinose, ribose, xylose, etc.) and/or modified sugars (e.g., 2′-fluororibose, 2′-deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, poly(dextrose), and fructose. In some embodiments, glycosaminoglycans are considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

The term “porogen” as used herein, refers to a chemical compound that may be part of the inventive composite and upon implantation/injection or prior to implantation/injection diffuses, dissolves, and/or degrades to leave a pore in the osteoimplant composite. A porogen may be introduced into the composite during manufacture, during preparation of the composite (e.g., in the operating room), or after implantation/injection. A porogen essentially reserves space in the composite while the composite is being molded but once the composite is implanted the porogen diffuses, dissolves, or degrades, thereby inducing porosity into the composite. In this way porogens provide latent pores. In certain embodiments, the porogen may be leached out of the composite before implantation/injection. This resulting porosity of the implant generated during manufacture or after implantation/injection (i.e., “latent porosity”) is thought to allow infiltration by cells, bone formation, bone remodeling, osteoinduction, osteoconduction, and/or faster degradation of the osteoimplant. A porogen may be a gas (e.g., carbon dioxide, nitrogen, or other inert gas), liquid (e.g., water, biological fluid), or solid. Porogens are typically water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade quickly under physiological conditions. Exemplary polymers include polyethylene glycol, poly(vinylpyrollidone), pullulan, poly(glycolide), poly(lactide), poly(lactide-co-glycolide), other polyesters, and starches. In certain embodiments, bone particles utilized in provided composites or compositions may act as porogens. For example, osteoclasts resorb allograft and make pores in composites.

In some embodiments, porogens may refer to a blowing agent (i.e., an agent that participates in a chemical reaction to generate a gas). Water may act as such a blowing agent or porogen.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (s), defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation/injection. It will be appreciated by these of ordinary skill in the art that the porosity of a provided composite or composition may change over time, in some embodiments, after implantation/injection (e.g., after leaching of a porogen, when osteoclasts resorbing allograft bone, etc.). For the purpose of the present disclosure, implantation/injection may be considered to be “time zero” (To). In some embodiments, the present invention provides composites and/or compositions having a porosity of at least about 30%, at least about 40%, at least about 50%, at least about 60%, at least about 70%, at least about 80%, at least about 90% or more than 90%, at time zero. In certain embodiments, pre-molded composites and/or compositions may have a porosity of at least about 30%, at least about 40%, at least about 50%, at least about 60%, at least about 70%, at least about 80%, at least about 90% or more than 90%, at time zero. In certain embodiments, injectable composites and/or compositions may have a porosity of as low as 3% at time zero. In certain embodiments, injectable composites and/or compositions may cure in situ and have a porosity of at least about 30%, at least about 40%, at least about 50%, at least about 60%, at least about 70%, at least about 80%, at least about 90% or more than 90% after curing.

The term “scaffold” as used herein refers to a substance that can be used to treat tissue and/or a wound. In some embodiments the scaffold or graft is a material that can be placed on or near tissue to be treated. The terms “composite”, “scaffold”, and “graft” may be used interchangeably herein to refer to embodiments of the presently-disclosed subject matter.

The term “setting time” as used herein, is approximated by the tack-free time (TFT), which is defined as the time at which the material could be touched with a spatula with no adhesion of the spatula to the foam. At the TFT, the wound could be closed without altering the properties of the material.

The term “small molecule” as used herein, is used to refer to molecules, whether naturally-occurring or artificially created (e.g., via chemical synthesis), that have a relatively low molecular weight. In some embodiments, small molecules have a molecular weight of less than about 2,500 g/mol, for example, less than 1000 g/mol. In certain embodiments, small molecules are biologically active in that they produce a local or systemic effect in animals, such as mammals, e.g., humans. In certain embodiments, a small molecule is a drug. In certain embodiments, though not necessarily, a drug is one that has already been deemed safe and effective for use by an appropriate governmental agency or body (e.g., the U.S. Food and Drug Administration).

The terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “tissue” is used herein to refer to a population of cells, generally consisting of cells of the same kind that perform the same or similar functions. The types of cells that make the tissue are not limited. In some embodiments tissue is part of a living organism, and in some embodiments tissue is tissue excised from a living organism or artificial tissue. In some embodiments tissue can be part of skin, bone, an organ or the like.

The term “transformation” as used herein, describes a process by which a material is removed from an implant site and replaced by host tissue after implantation. Transformation may be accomplished by a combination of processes, including but not limited to remodeling, degradation, resorption, and tissue growth and/or formation. Removal of the material may be cell-mediated or accomplished through chemical processes, such as dissolution and hydrolysis.

The terms “treatment” or “treating” refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. For example, in some embodiments treatment refers to the healing bone tissue that is fractured and/or healing wounded skin tissue.

The term “wet compressive strength” as used herein, refers to the compressive strength of an osteoimplant after being immersed in physiological saline (e.g., phosphate-buffered saline (PBS), water containing 0.9 g NaCl/100 ml water, etc.) for a minimum of 12 hours (e.g., 24 hours). Compressive strength and modulus are well-known measurements of mechanical properties and is measured using the procedure described herein

The term “working time” as used herein, is defined in the ISO9917 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows. According to the present invention, the working time is measured by loading the syringe with the reactive composite and injecting <0.25 ml every 30s. The working time is noted as the time at which the material was more difficult to inject, indicating a significant change in viscosity.

The term “wound” as used herein refers to any defect, injury, disorder, damage, or the like of tissue. In some embodiments a wound can be a bone fracture. In some embodiments a wound is damaged skin or skin that must heal from a particular disorder.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Provided herein are thioketal (TK) polymers and long chain crosslinkers. In some embodiments, the TK polymer includes a carbobetaine poly(thioketal) (PTK) copolymer having the structure according to Formula I below:

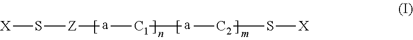

where a includes a thioketal; C₁ includes a zwitterionic monomer; C₂ includes poly(ethylene glycol) (PEG); X includes OH, NH₂, SH, hydroxyalkyl, aminoalkyl, or thioalkyl; Z includes —C₁—, —C₂—, or —C₁-a-C₂—; n is between 0 and 15; m is between 0 and 15; and if one of either n or m is 0 the other is at least 1. In some embodiments, a includes a thioketal having the following structure:

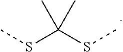

In some embodiments, the zwitterionic monomer of C1 includes one or more of the following structures:

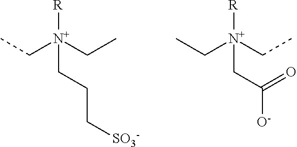

where R includes H, methyl, ethyl, isopropyl, or benzyl. In some embodiments, the PEG of C₂ includes the following structure:

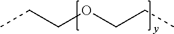

where y is between 0 and 2. In some embodiments, the alkyl portion of the hydroxyalkyl, aminoalkyl, or thioalkyl in X includes a (C₁-C₆) alkyl. For example, in one embodiment, X includes hydroxyethyl.

In some embodiments, n in the polymer according to Formula I is 0. In some embodiments, where n is 0, Z in the polymer according to Formula I is C₂. In some embodiments, where n is 0 and Z is C₂, X is hydroxyethyl. In such embodiments, examples of polymers according to Formula I include, but are not limited to:

| y = | Structure | Name |
|---|---|---|
| 0 | 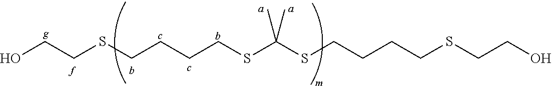 | EG0 |
| 1 | 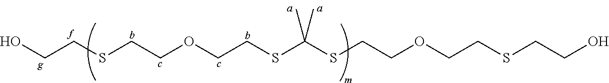 | EG1 |
| 2 | 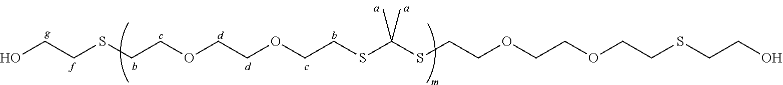 | EG2 |

Alternatively, in some embodiments, both n and m are greater than 0. In some embodiments, where n and m are both greater than 0, Z is —C₂—. In such embodiments, an example of a polymer according to Formula I includes, but is not limited to:

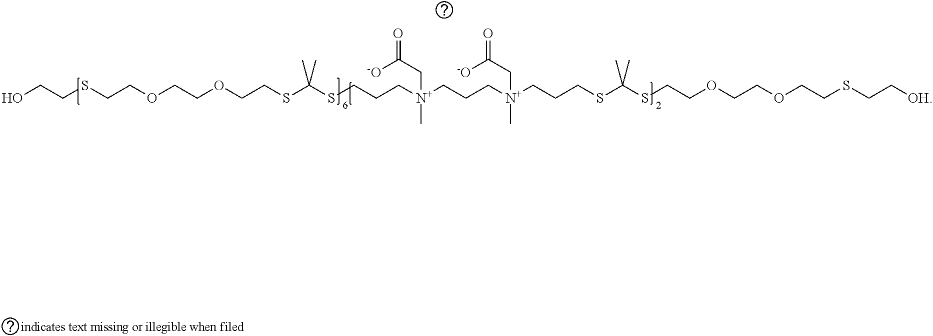

Additionally or alternatively, in some embodiments, where n and m are both greater than 0, Z is —C₁-a-C₂—. In such embodiments, an example of a polymer according to Formula I includes, but is not limited to:

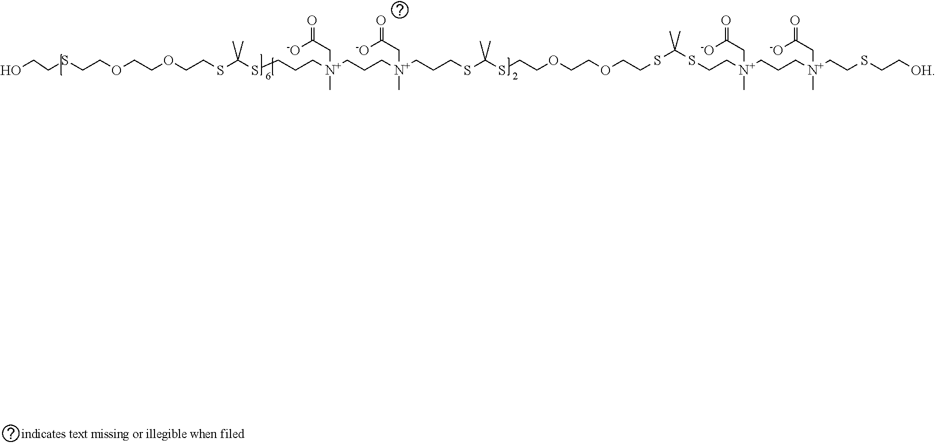

Although described herein primarily with respect to specific examples according to Formula I, as will be appreciated by those skilled in the art, the disclosure is not so limited and may include any other suitable combination of a, C₁, C₂, R, X, Z, n, m, and y, each of which is explicitly considered herein. For example, in some embodiments, a is thioketal, X is hydroxyethyl, Z is —C₂—, and C₂ is PEG, with any combination of R, C₁, n, m, and y. Other examples include, but are not limited to, polymers where a is thioketal, Z is —C₂—, C₂ is PEG, with any combination of R, X, C₁, n, m, and y; polymers where a is thioketal, Z is —C₁-a-C₂—, C₂ is PEG, with any combination of R, X, C₁, n, m, and y, or any other variation of elements.

In some embodiments, the TK polymer includes a PTK polymer having the structure according to Formula II below:

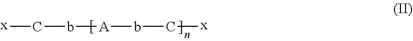

where A includes a thioketal crosslinker; b includes carbamate or carbonate; C includes poly(ethylene glycol) (PEG); x includes OH, NH₂, SH, hydroxyalkyl, aminoalkyl, or thioalkyl; and n is between 1 and 10. In some embodiments, A includes a thioketal having the following structure:

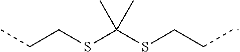

In some embodiments, the carbamate and/or carbonate of b includes the following structure:

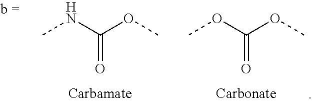

In some embodiments, the PEG of C includes the following structure:

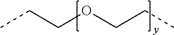

where y is between 3 and 15. In some embodiments, y is between 3 and 15, between 4 and 15, between 5 and 15, between 6 and 15, between 7 and 15, at least 7, or any combination, sub-combination, range, or sub-range thereof. In some embodiments, the alkyl portion of the hydroxyalkyl, aminoalkyl, or thioalkyl in X includes a (C₁-C₆) alkyl. For example, in one embodiment, X includes hydroxyethyl. For example, in some embodiments, the PTK polymer according to Formula II has the following structure:

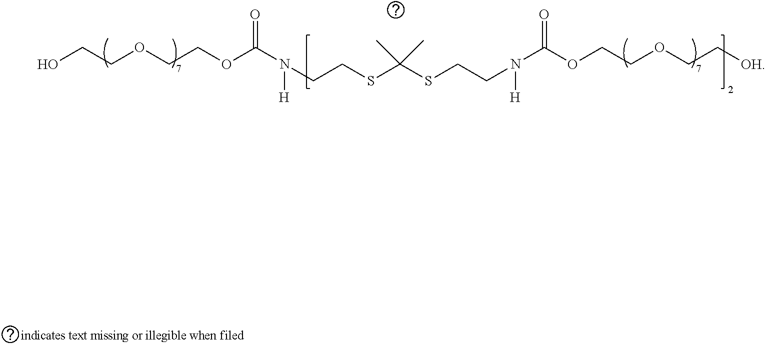

Although described herein primarily with respect to specific examples according to Formula II, as will be appreciated by those skilled in the art, the disclosure is not so limited and may include any other suitable combination of A, b, C, x, n, and y, each of which is explicitly considered herein.

In some embodiments, the TK polymer includes a psar-polypeptoid thioketal polymer having the structure according to Formula III below:

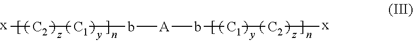

where A includes a thioketal crosslinker; b includes 2° or 3° amide; C₁ includes a poly(peptide); C₂ includes a poly(peptoid); x includes OH, NH₂, SH, hydroxyalkyl, aminoalkyl, or thioalkyl; y is from about 0 to about 1; z is from about 1 to about 0; and n is between 1 and 15. In some embodiments, A includes a thioketal having the following structure:

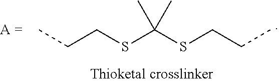

In some embodiments, the amide of b includes the following structure:

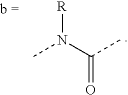

In some embodiments, the poly(peptide) of C₁ includes the following structure:

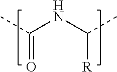

where R includes H, methyl, ethyl, isopropyl, or benzyl. In some embodiments, the poly(peptoid) of C₂ includes the following structure:

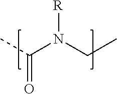

where R includes H, methyl, ethyl, isopropyl, or benzyl. In some embodiments, the alkyl portion of the hydroxyalkyl, aminoalkyl, or thioalkyl in X includes a (C₁-C₆) alkyl. For example, in one embodiment, X includes hydroxyethyl. For example, in some embodiments, the TK polymer according to Formula III has the following structure:

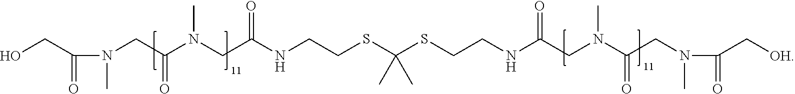

Although described herein primarily with respect to specific examples according to Formula II, as will be appreciated by those skilled in the art, the disclosure is not so limited and may include any other suitable combination of A, b, C₁, C₂, R, x, n, y, and z, each of which is explicitly considered herein.

Also provided herein are methods of synthesizing the TK polymers according to Formulas I-III. As discussed in detail below, the TK bonds can be formed during the PTK diol polymerization or can be integrated into an initiator/precursor utilized in the formation of the PTK diol. In some embodiments, the method of synthesizing the TK polymer according to Formula I includes forming the thioketal bond during the step-growth (A/B type) condensation polymerization reaction. For example, in some embodiments, the TK polymer of Formula I is synthesized according to the schematic below:

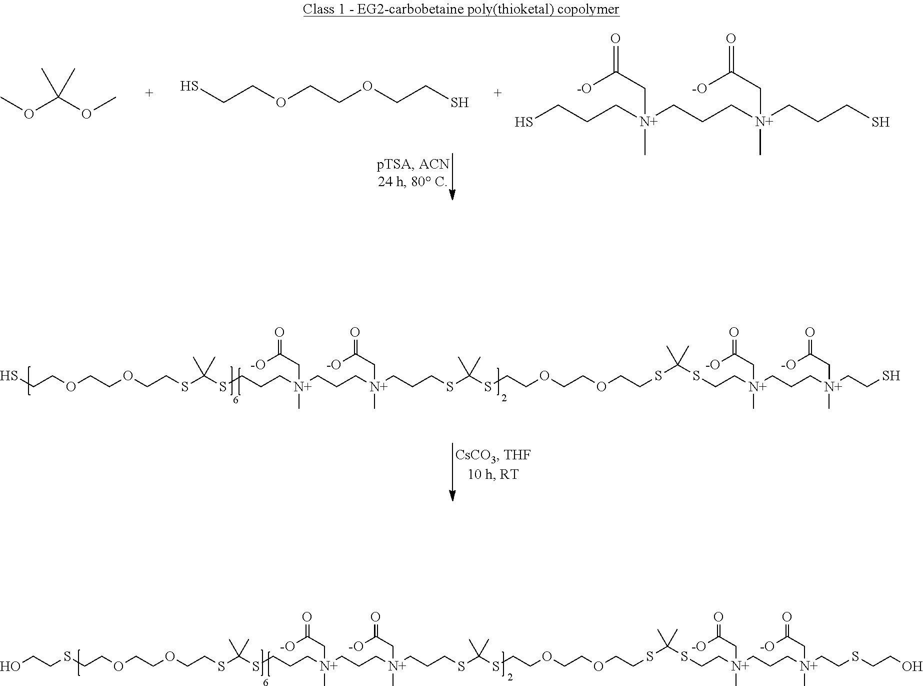

In contrast to existing polymers that are formed through polymerization of alkyl, PEG, and dimethoxypropane (DMP) monomers, which are more hydrophobic due to limitations with generation of materials using dithiol monomers with more than 2 ethylene glycol units (i.e., larger than EG2 in size), the polymers disclosed herein include a zwitterionic monomer that increases the hydrophilicity of the polymer. As will be appreciated by those skilled in the art from the structures and discussion herein, these polymers may be synthesized from polymerization of zwitterionic monomers or copolymerization of zwitterionic monomers and other dithiols (e.g., PEG).

In some embodiments, the method of synthesizing the TK polymer according to Formula II includes generating the polymer from a preformed short-chain TK crosslinker (e.g., TK diamine, diol, or diacid) conjugated with a mono or heterobifunctional macromere such as, but not limited to, NPC-PEG-NPC, NPC-PEG-OH [NPC (nitrophenylcarbonate) is an amine reactive group]. For example, in some embodiments, the TK polymer of Formula II is synthesized according to the schematic below:

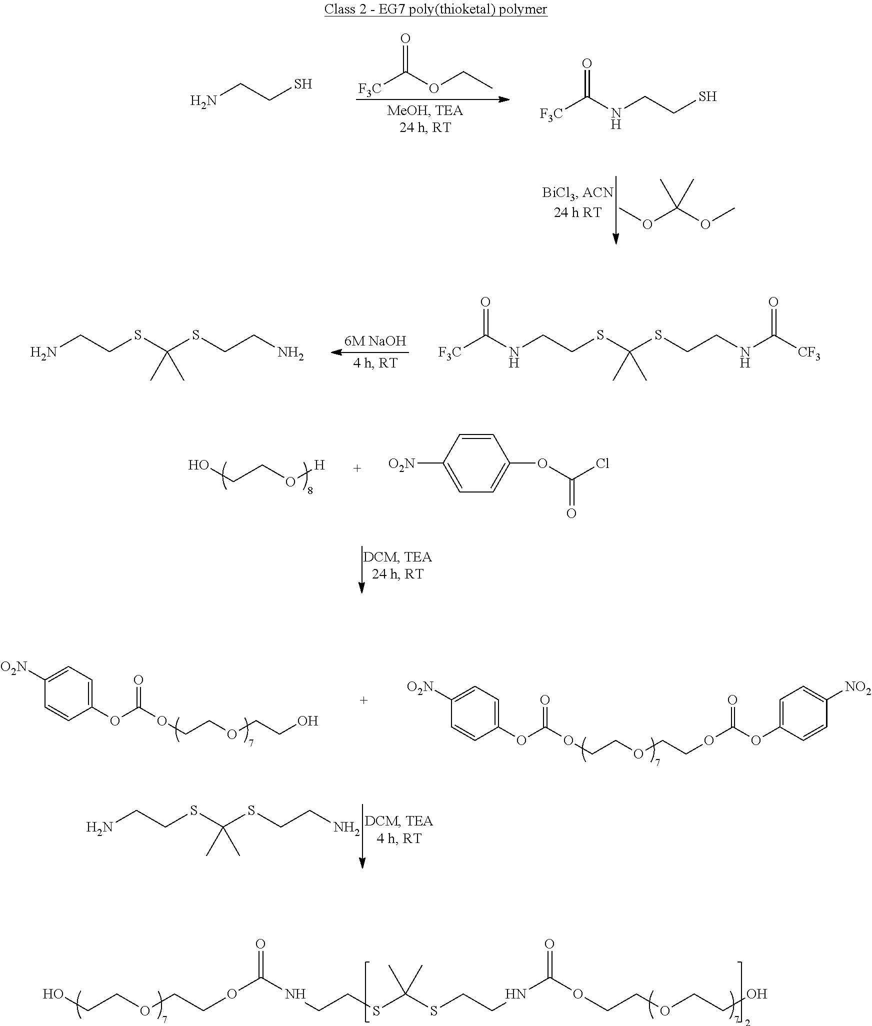

In contrast to existing PTK polymers, in some embodiments, the polymer according to Formula II includes a diamine in the backbone. Additionally, this process can be designed to yield just 1 or multiple backbone TK units per resultant diol. The number of TK units (and diol molecular weight) can be varied based on the ratio of hetero- vs homo-bifunctional macromers used in the initial step-growth addition reaction. The heterobifunctional macromers serve as “end-cappers”. If a 2:1 ratio of heterobifunctional macromer:TK crosslinker is reacted, it will yield “long chain TK diol” with one TK unit in the backbone. The homobifunctional PEG macromers can bridge two TK diamine crosslinker precursors resulting in longer chains with multiple TK units in the backbone of the PTK diol products to give an alternating ABA polymeric structure.

By generating the polymers from a preformed short-chain TK crosslinker, as opposed to previous synthesis methods where the thioketal bond is formed during the step-growth (A/B type) condensation polymerization reaction, the method disclosed herein permits the inclusion of additional ethylene glycol (EG) units. For example, whereas the previous methods were limited to generation of materials using dithiol monomers with 2 or less EG units (e.g., due to poor polymerization control/yield and/or cycle formation vs forming linear chains), the methods disclosed herein permit the formation of polymers having more than 2 EG units. Accordingly, in some embodiments, the polymers include at least 3 EG units, at least 4 EG units, at least 5 EG units, at least 6 EG units, or at least 7 EG units. The additional EG units provide increased hydrophilicity as compared to the relatively hydrophobic PTK diols with 2 or less EG units, which provides improved wound healing over existing PTK diols.

In some embodiments, the method of synthesizing the TK polymer according to Formula III includes ring-opening polymerization of N-carboxyanhydride or N-thiocarboxyanhydride peptide or peptoid monomers from small, pre-formed TK-diamine initiator to produce poly(peptide)s or N-substituted poly(peptoid)s. For example, in some embodiments, the TK polymer of Formula III is synthesized according to the schematic below:

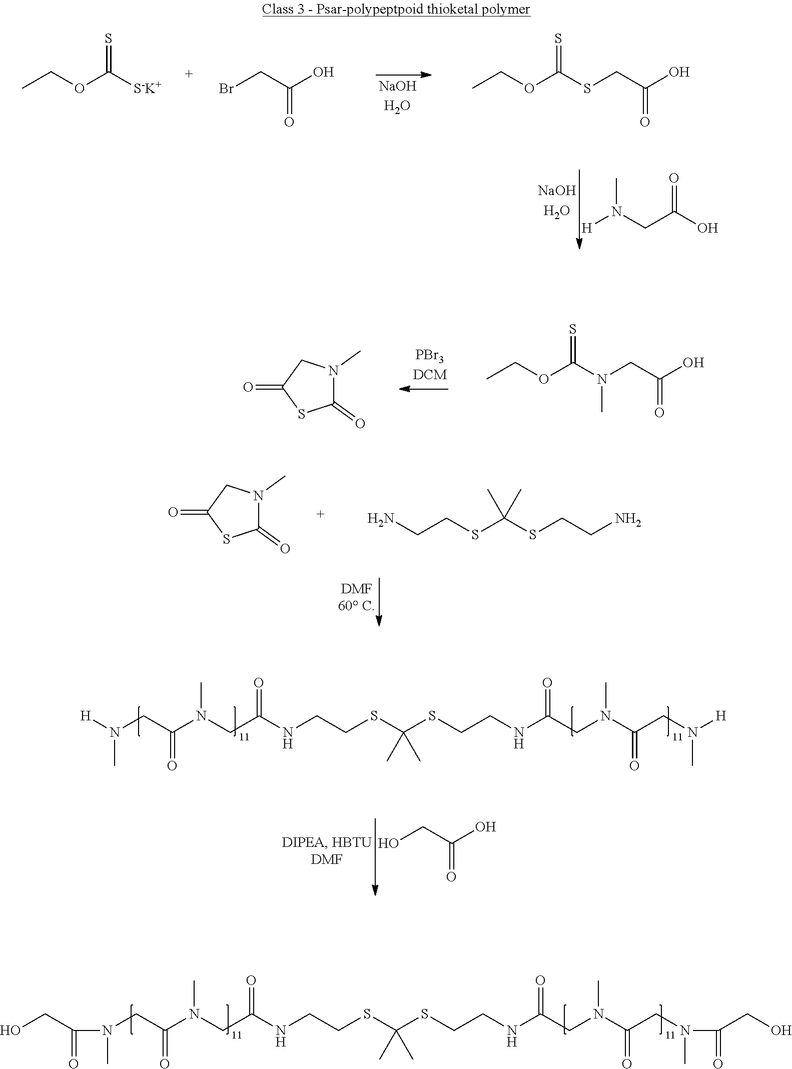

In some embodiments, the TK polymer according to one or more of the embodiments disclosed herein serve as precursor polymers required for the fabrication of polyurethane foams. In some embodiments, polyurethane scaffolds can be made by reacting the TK polymer with a polyisocyanate.

Suitable polyisocyanates or multi-isocyanate compounds include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. For example, in some embodiments, using lysine triisocyanate as a crosslinker, the TK polymers may be used to fabricate three-dimensional scaffolds with interconnected porous structure. Other suitable polyisocyanates used herein also include aromatic polyisocyanates. In some embodiments, the polyisocyanates include approximately 10 to 55% NCO by weight (wt % NCO=100*(42/Mw)). In some embodiments, polyisocyanates include approximately 15 to 50% NCO.

The isocyanates disclosed herein can react with the terminal functional group (e.g., hydroxyl) of the TK polymer. The relative rates of these reactions determine the scaffold morphology, working time, and setting time. The thioketal polymer and polyisocyanate can be mixed in any proportion that results in a scaffolds having desired characteristics in terms of strength, flowability, and the like. For example, the scaffolds can comprise about 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, 80 mol %, 85 mol %, 90 mol %, 95 mol %, TK polymer or polyisocyanate. Similarly to the PUR scaffolds discussed above, the ratio of TK polymer to polyisocyanate can be optimized so there is a deficiency or an excess of the number of reactive groups of the TK polymer in relation to the NCO equivalents on the polyisocyanate. On the other hand, in some embodiments there is an approximately stoichiometric ratio of TK polymer functional groups to NCO groups on the polyisocyanates. The molecular weights of the TK polymer, the subunits of the TK polymer, and/or the polyisocyanates can also be varied to manipulate the degradability, density, and other characteristics of the scaffolds. In some embodiments the present scaffolds are comprised of a network of TK polymers and polyisocyanates that are cross-linked (i.e., covalently bound) through a curing process. In some embodiments, pores in bone/polyurethanes composites in the present invention are interconnected and have a diameter ranging from approximately 50 to approximately 1000 microns.

In some embodiments, these PTK-UR scaffolds may be used for wound treatment. For example, in some embodiments, the scaffolds formed from the TK polymers promote tissue integration and vascularization within the wound. In some embodiments, the scaffolds formed from the TK polymers downregulate inflammatory genes in treated wounds, as compared to more hydrophobic PTK-Ur scaffold variants. In some embodiments, the scaffolds provide upregulation of genes associated with extracellular matrix deposition and remodeling.

In some embodiments, the TK polymer according to one or more of the embodiments disclosed herein is a fully synthetic, resorbable, porous poly(thioketal) urethane based dermal substitute. Additionally, in some embodiments and in contrast to existing products, the TK polymer is fully synthetic and does not contain any biological materials, such as collagen, making it suitable for applications in patients who have known allergies to bovine and porcine derived products. Unlike some ECM based products, the TK polymers disclosed herein do not undergo rapid matrix metalloprotease driven degradation that results in spontaneous contraction of the wounds. Furthermore, the TK polymer scaffolds have been shown to degradable through natural biological cues (reactive oxygen species), creating degradation and resorption kinetics to match that of native skin tissue regeneration, unlike current commercially available synthetic dermal substitutes that are made of polyesters which undergoes nonspecific hydrolytic degradation, resulting in an autocatalytic degradation profile (acidic degradation byproducts accelerate material degradation) that can cause sudden collapse and reduction in mechanical support provided by the scaffold. This degradation also avoids the extended persistence of scaffolds, such as that shown with polyester urethane based scaffolds which have exhibited scaffolds remnants up to 9 months post treatment that can be associated with foreign body response resulting in pain and discomfort for the patients.

In some embodiments, the TK polymers disclosed herein may be used to produce scaffolds for the treatment of full thickness excisional wounds. Additionally or alternatively, the TK polymers may be used for the treatment of second-degree burns, surgical and traumatic wounds, diabetic ulcers, venous ulcers, and pressure ulcers. Furthermore, the scaffolds formed with the TK polymers disclosed herein could be used along with Negative Pressure Wound Therapy (NPWT) or combined with drugs or antibiotics (integration of silver for example).

Further provided herein, in some embodiments, are methods of forming scaffolds form the polymers disclosed herein. In some embodiments, forming the scaffold includes reactive liquid molding. For example, in some embodiments, the polymer disclosed herein are mixed with a pore opener (e.g., calcium stearate), a catalyst (e.g., TEGOAMIN33 available from Evonik), a blowing agent (e.g., water), and a pore stabilizer (e.g., sulfated castor oil) for any suitable amount of time (e.g., 30 seconds at 3300 rpm). Next, lysine triisocyanate (LTI) is added and mixed for any suitable amount of time (e.g., 30 seconds at 3300 rpm). The mixture is then allowed to rise, set, and harden before the scaffolds are demolded and trimmed to shape. In some embodiments, isocyanate: hydroxyl ratio (NCO OH) is set to 115:100, where the OH equivalents are obtained from polyol's molecular weight and OH number obtained from ¹⁹F NMR analysis. Example compositions are shown in Table 1 in the Examples below.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

### Example 1

This Example describes the synthesis and characterization of poly(thioketal) polyols having increased ethylene glycol content. Poly(thioketal) polymers fall under an umbrella of stimuli responsive materials that have been shown to degrade in the presence of reactive oxygen species (ROS). Elevated levels of ROS in chronic wounds have made PTK polymers an attractive and cost-effective candidate for engineering biomaterials that can be used as dermal substitutes. PTK urethane scaffolds have been shown to degrade in the presence of hydrogen peroxide, superoxide, and hydroxyl radicals more appropriately tying scaffold degradation to ROS production during cellular infiltration, growth, and metabolic activity.

Reactive Liquid Molding of Isocyanates and Poly(Thioketal) Polyols Results in Covalently Crosslinked 3D Network with Interconnected Pores that Facilitate Tissue Infiltration (FIG. 3).

Fabrication of porous PTK-UR scaffolds. Trifunctional isocyanates react with hydroxyl groups on divalent polyols in the presence of a catalyst resulting in a network of covalent polyurethane bonds. Use of water as a blowing agent helps achieve a porous structure within the scaffolds that promote tissue infiltration and integration into the wound. The present inventors have synthesized a family of PTK polyols with increasing ethylene glycol (EG content) in their polymer backbone. It is believed that increasing PTK diol hydrophilicity leads to an increase in the hydrophilicity of bulk polyurethane scaffold, increased ROS reactivity, promotes better cell growth/infiltration, and increases solubility and clearance of breakdown products.

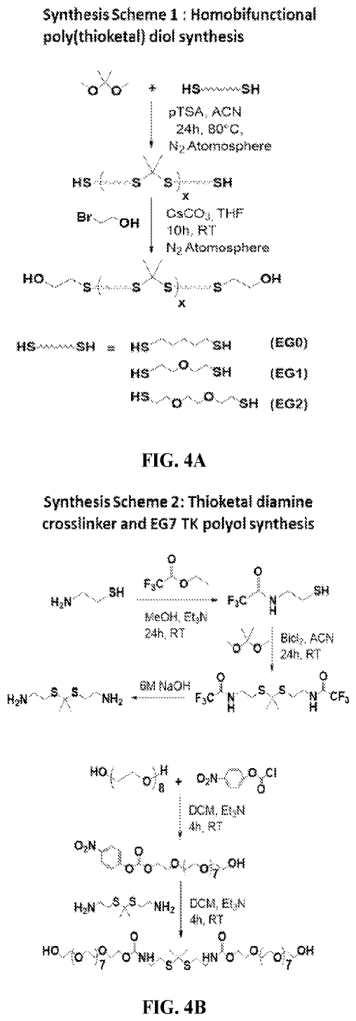

### Synthesis of EG0, EG1, EG2 and EG7 PTK Diols (FIGS. 4A-B).

EG0, EG1 and EG2 were synthesized through condensation polymerization of butane dithiol (BDT), 2-mercaptoethyl ether (MEE) and 2,2′-(Ethylenedioxy) diethanethiol (EDDT) respectively (FIG. 4A). The dithiol monomers were polymerized with 2,2-dimethoxypropane (DMP) in the presence of pTSA as a catalyst. Hydroxyl functionalization of PTK dithiols was carried out using 2-bromoethanol in the presence of CsCO₃ as a catalyst.

EG7 PTK diols were synthesized in a two-step process (FIG. 4B). First, a short diamine thioketal crosslinker was synthesized. Cysteamine was reacted with ethyl trifluoroacetate in the presence of triethylamine. This trifluoroacetate protected cysteamine was further reacted with DMP in the presence of BiCl₃ catalyst to obtain thioketal groups. Second, an amine reactive PEG 400 (Mn=400 Da) derivative was synthesized. PEG 400 was dried and reacted with nitrophenyl chloroformate to obtain a monofunctionalized PEG 400 phenylnitrocarbonate (PEG400 NPC). PEG 400 NPC was then reacted with thioketal diethylamine (TK crosslinker) to obtain EG7 PTK diol with terminal hydroxyl groups.

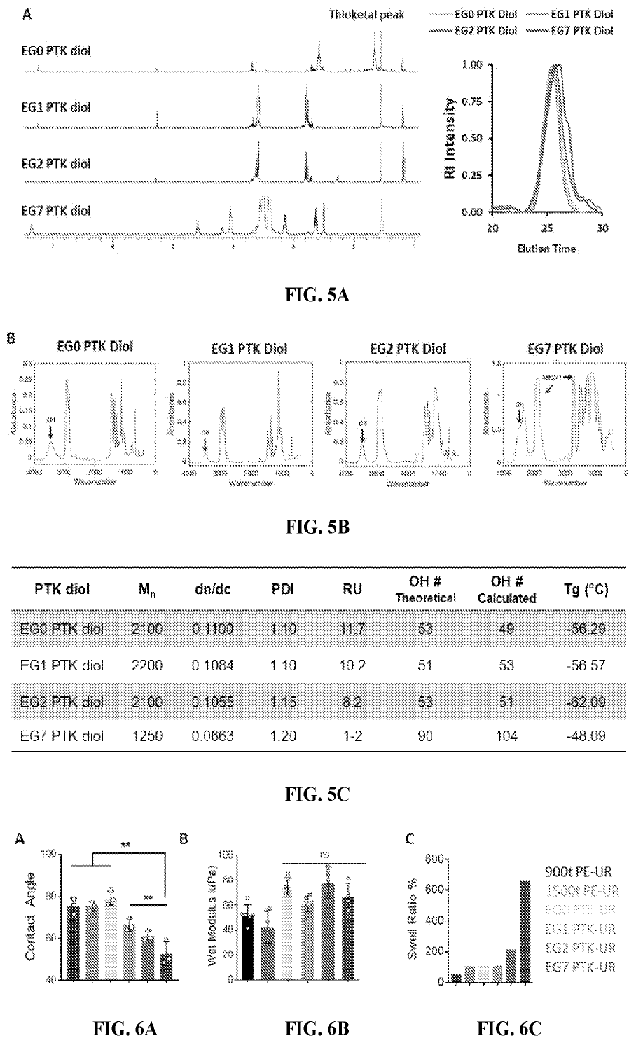

### PTK Diol Characterization (FIGS. 5A-C).

PTK diols were successfully synthesized and characterized using ¹H NMR to validate the presence of ROS response thioketal groups present in the polymer backbone (FIG. 5A). Size and dispersity of the resulting PTK diols was verified using GPC. All PTK diols demonstrated relatively monodisperse populations with low polymer dispersity (D). Presence of terminal hydroxyl groups on the PTK diols were verified through ATR-FTIR (FIG. 5B). The diagnostic hydroxyl (OH) absorbance peak is seen at 3400 cm⁻¹ (black arrow), and was present in all four polymers. Quantification of terminal hydroxyls using ¹⁹F NMR closely matched theoretical values (FIG. 5C).

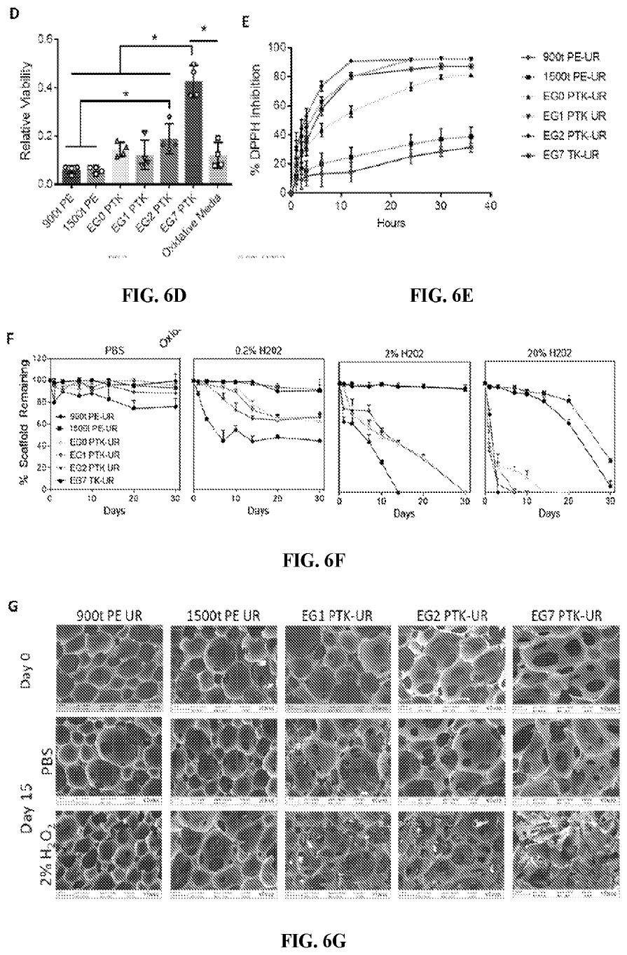

### Increased EG Content in PTK Polyol Allows for Modulation of Physical and Chemical Properties of PTK-UR Scaffolds (FIGS. 6A-G).

Porous PTK-UR scaffolds were fabricated using E0, EG1, EG2 and EG7 PTK diols and lysine triisocyanate (LTI). PTK-UR scaffold variants were benchmarked against 900 and 1500 polyester triol-LTI based polyester urethane scaffolds.

Hydrophilicity and wettability of polyurethane scaffolds were characterized through contact angle measurements. EG7 PTK-UR films exhibited the lowest contact angle of 500 with higher spreading of the water droplet whereas EG0 PTK-UR films were associated with higher contact angles of 80°, similar to that of 900t and 1500t PE films. The water contact angles of EG1 and EG2 was recorded as 70° and 60° respectively. Through progressive incorporation of EG repeats in the polymeric backbone, we were able to increase the overall hydrophilicity of the PTK-UR scaffolds.

Incorporation of increasing EG repeats in the PTK polyol monomeric backbone did not significantly alter the mechanical properties of the resulting urethane scaffolds. The Young's modulus of fully hydrated scaffolds ranged between 60-80 kPa.

To assess the degradation properties of PTK-UR scaffolds, 10 mg pieces were incubated in either PBS, 0.2, 2 or 20% H₂O₂ solutions, and the resulting mass loss was measured for up to 30 days. PU scaffolds fabricated from PTK diols demonstrated dose dependent degradation in oxidative media but were relatively stable in hydrolytic media. Higher mass loss was observed in EG7 PTK-UR scaffolds compared to EG0, EG1 and EG2 PTK urethane scaffolds incubated in 0.2 and 2% H₂O₂.

DPPH was used as a surrogate molecule to measure ROS scavenging capacity of PTK and PE UR scaffolds. Scaffold pieces were incubated in 200 μM DPPH solutions and compared to untreated DPPH solution to calculate relative inhibition of DPPH activity. PTK-UR scaffolds showed significantly higher DPPH inhibition compared to PE-UR scaffolds. No differences in DPPH inhibition were observed within the PTK-UR groups. To further resolve the protective capacity of PTK diols, 3T3 fibroblasts were sequentially treated with 50 μM H₂O₂ solution followed by PTK diols. EG7 PTK diol treatment groups demonstrated highest protection against cell death compared to EG0, EG1, EG2 PTK and PE treatment groups.

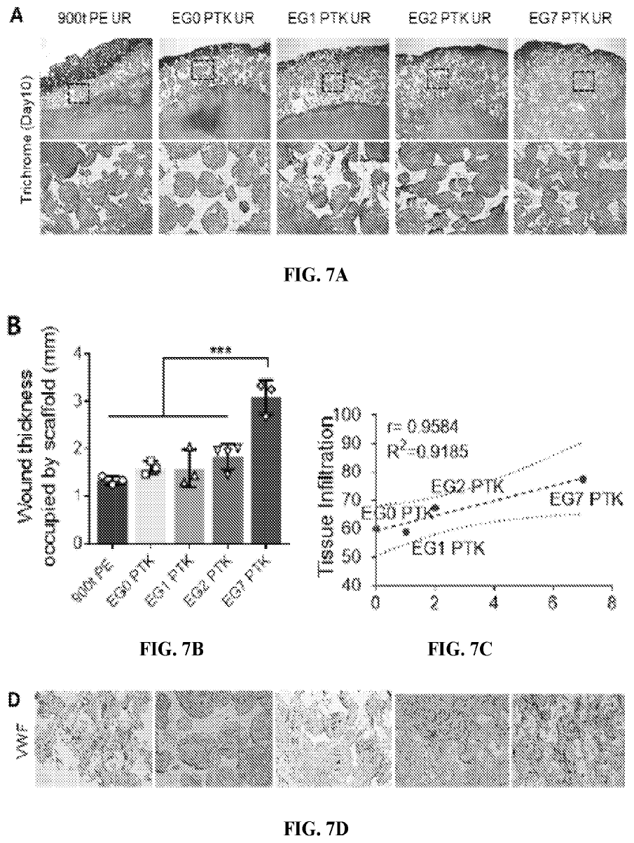

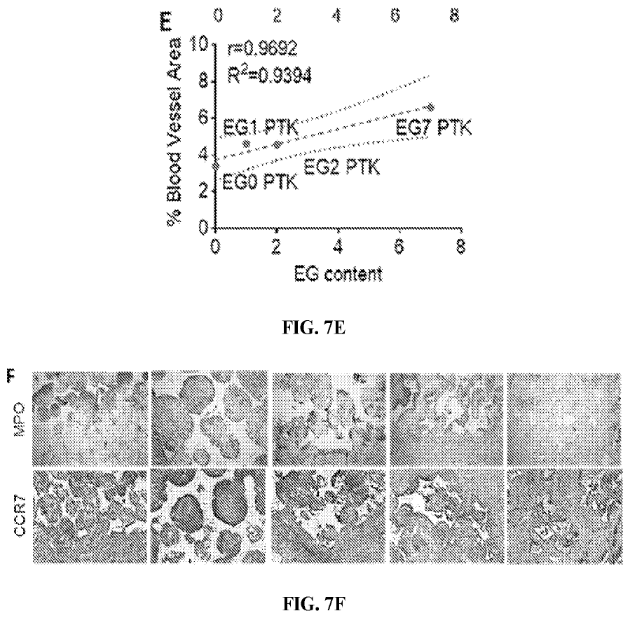

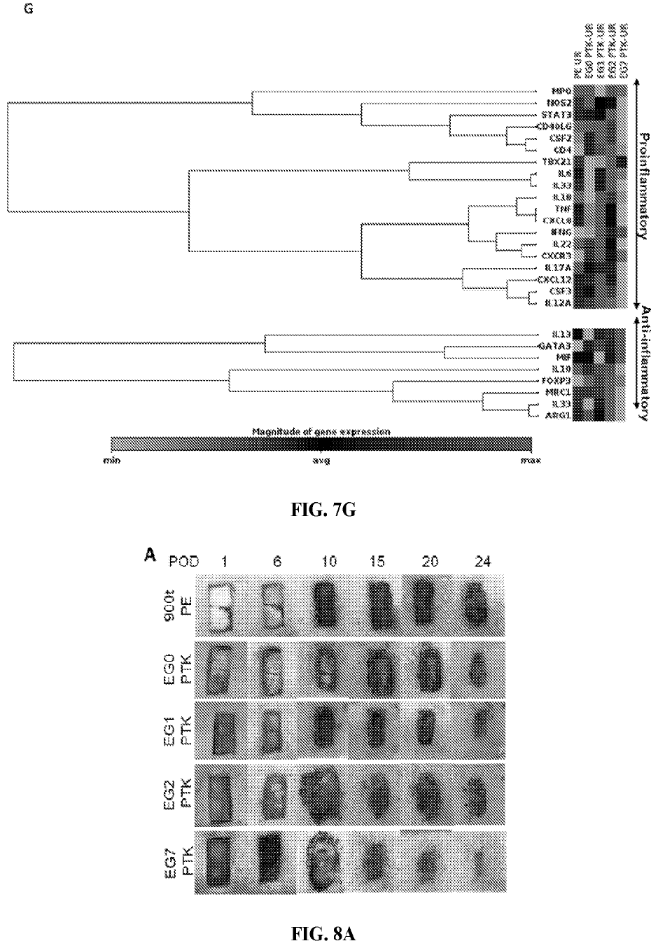

### EG7PTK-UR Scaffolds Facilitate Tissue Integration and Neovascularization 10 Days Post Implantation in Porcine Full Thickness Wounds (FIGS. 7A-G).

To investigate scaffold mediated cell infiltration in vivo, polyurethane scaffolds were implanted in excisional wounds created on dorsal region of pigs. Tissue infiltration into the pores of the scaffolds was observed in all PU treatments. EG7 PTK-UR scaffolds demonstrated the highest tissue infiltration compared to 900t PE and EG0, EG1, EG2 PTK scaffolds. In addition to infiltration into the pores, EG7 PTK-UR scaffold pieces were uniformly distributed throughout the thickness of the wounds compared to other scaffold treatments, suggesting robust bulk integration of scaffolds in the wound bed. Positive correlation between tissue infiltration and EG content in the polymeric backbone was observed.

Host endothelial recruitment and formation of blood vessels was also observed within the scaffold infiltrating granulation tissue. Quantification of blood vessels within this region showed a positive correlation between EG repeats and vascular density. We were able to show that by increasing the overall hydrophilicity of the PTK-UR scaffolds through increasing the number of EG repeat units in PEG (/using longer PEGs), we could increase scaffold-tissue integration and vascularization within the implant.

To assess biomaterial associated inflammation in excisional wounds, we performed immunohistology of scaffold infiltrating granulation tissue 10 days post implantation. Myeloperoxidase (MPO) staining significantly decreased across the EG PTK diol series with minimal staining observed in EG7 scaffold treatments compared to EG0 PTK-UR indicating decreased neutrophil infiltration and inflammation within the wound. Furthermore, EG7 PTK-UR scaffold application showed downregulated expression of inflammatory genes such as CSF2, TNFa, IL1B, NOS2, TBX21 and IL12A. We also observed upregulation of wound healing associated genes such as GATA3, IL3 and MIF indicating a transition from an inflammatory to a remodeling/healing phase in tissue repair.

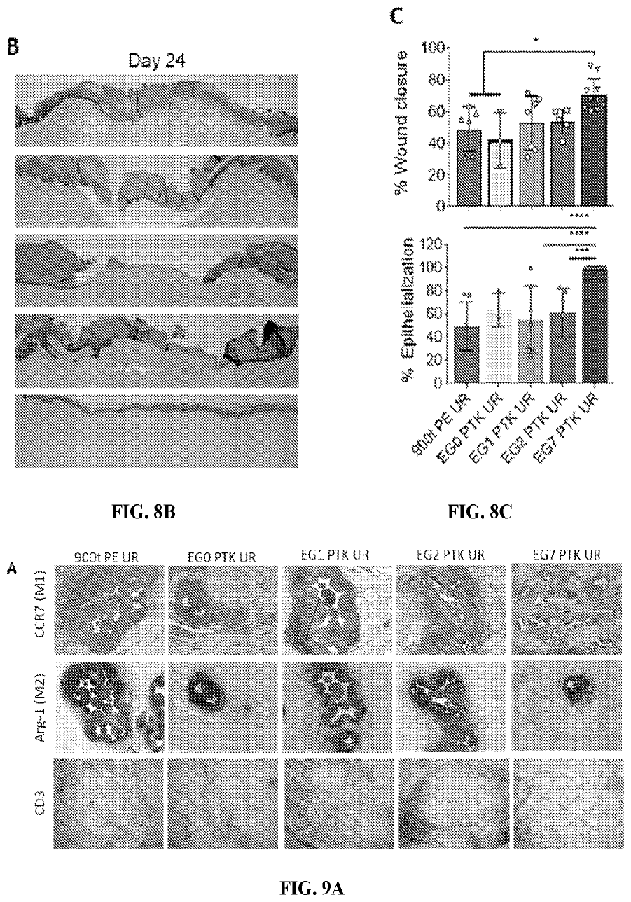

Increased Rate of Wound Closure and Re-Epithelialization of Porcine Wounds Treated with EG7PTK-UR Help Restore Barrier Function of Epithelial Epidermal Layer (FIGS. 8A-C).

To resolve wound healing outcomes of polyol chemistries, we looked at the tissue response to implanted polyurethane scaffolds in porcine excisional wounds. Wound closure was evaluated over a period of 24 days post-surgery. Highest wound closure (72%) was observed in EG7 PTK treated wounds compared to only 40% and 50% wound closure observed in EG0 PTK and 900t PE treated wounds. Almost all EG7 PTK treated wounds were fully re-epithelialized with a uniform and thin layer of CytoK14+ epithelial cells indicating complete restoration of epithelial barrier. In comparison, incomplete and hyperproliferative epidermis was associated with 900t PE, EG0, EG1 and EG2 PTK treated wounds indicating inflammation and poor epidermal migration.

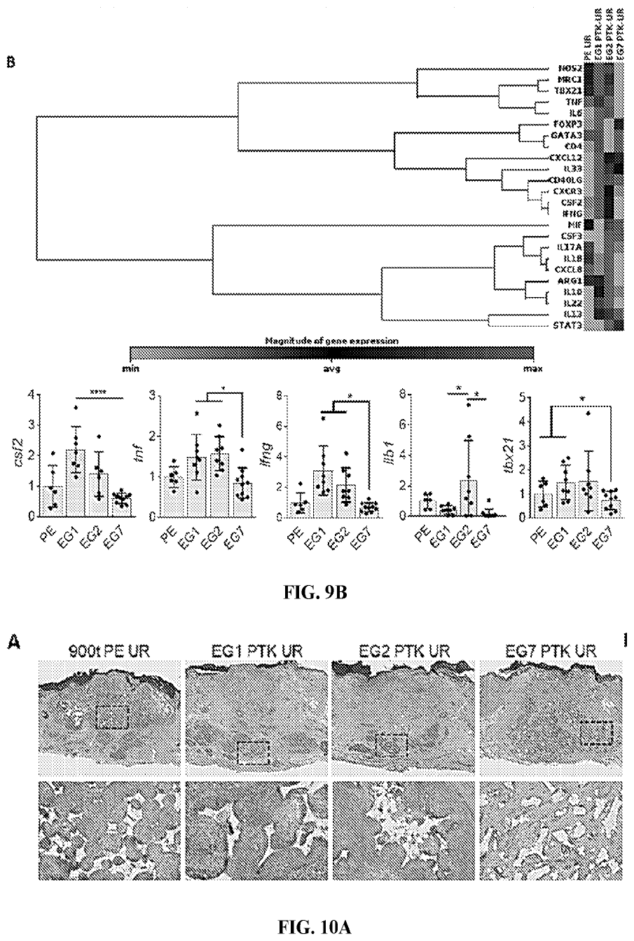

Decreased Inflammatory Response Associated with EG7PTK-UR Treated Skin Wounds Promote Functional Tissue Restoration (FIGS. 9A-B).

We investigated long term inflammatory response associated with polyurethane scaffolds in the wound bed. We observed significant decrease in the intensity of CCR7, a pro-inflammatory marker present primarily on activated pro-inflammatory macrophages around EG7 PTK scaffold remnants compared to more hydrophobic PTK and PE urethane treatments indicating decreased inflammatory response associated with scaffold remnants.

Gene expression analysis of granulation tissue showed downregulation of inflammatory genes in the EG7 PTK-UR treated wound such as NOS2, TBX21, TNFa, IL1b, IL6, CSF2, IFNg compared to more hydrophobic variants. In addition, upregulation of GATA3, IL-13 and MIF, genes associated with anti-inflammatory response was observed in EG7 treatments compared to EG1 and EG2 PTK urethane scaffolds.

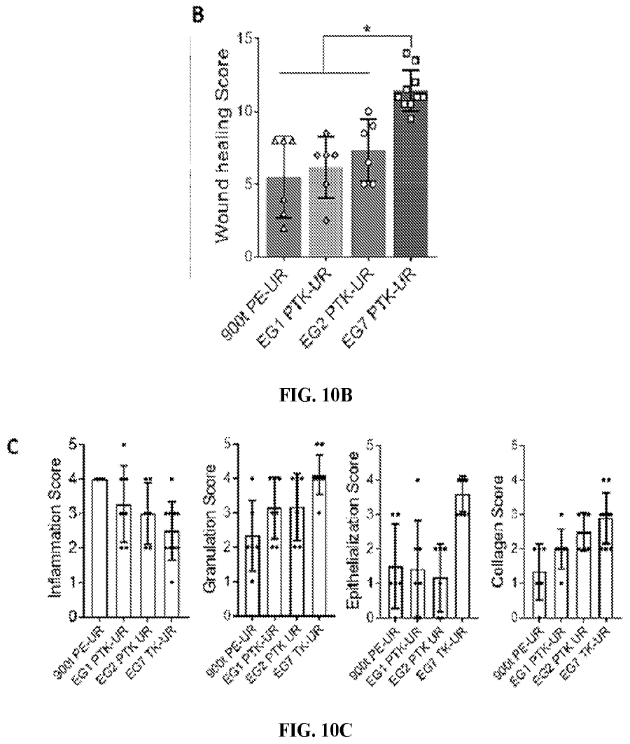

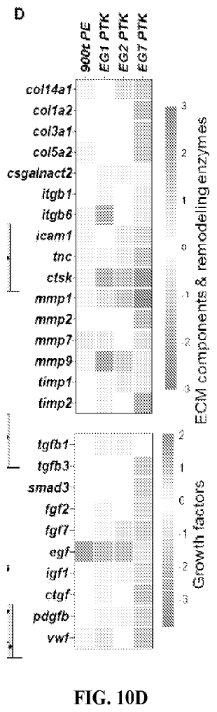

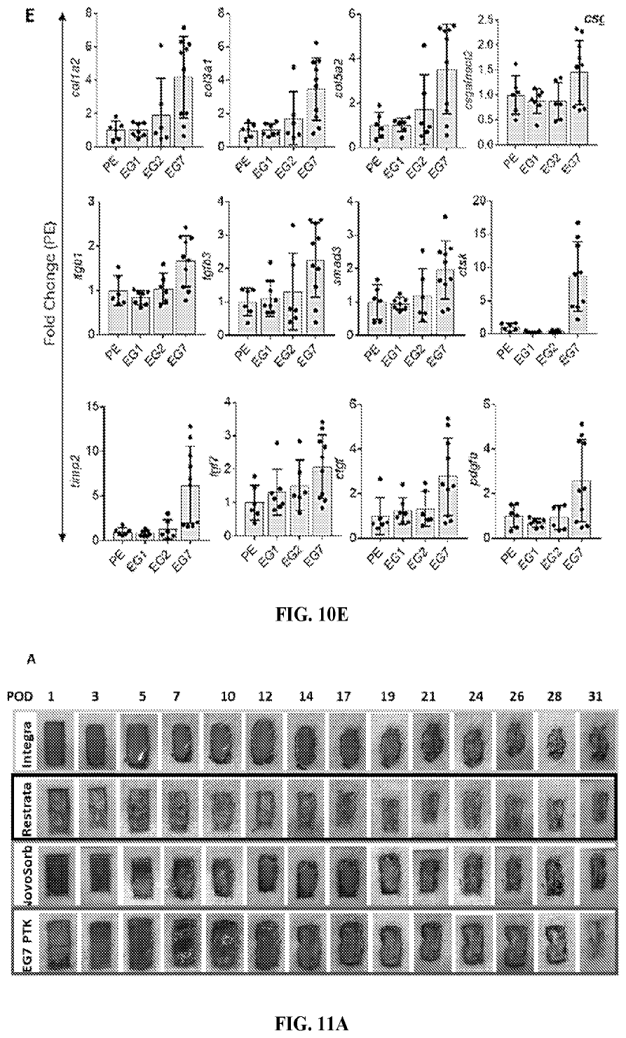

### EG7 PTK UR Scaffolds Allow Robust Tissue Regeneration and Repair Compared to More Hydrophobic PTK-UR Variants In Vivo Through Increased ECM Deposition and Remodeling (FIGS. 10A-E).

In addition to biomaterial associated inflammation, we investigated the effects of scaffold chemistry on tissue repair and wound restoration. Treatment blinded histomorphometry analysis of trichrome and H&E stained tissue sections was used to assign wound healings scores to granulation tissue formation, epithelialization, collagen deposition, vascularization and inflammation. EG7 PTK-UR treated wound outperformed PE-UR, EG1 and EG2 PTK-UR treatments with a higher overall wound healing score. EG7 treated wounds showed higher granulation tissue formation and collagen deposition along with robust re-epithelialization of the epidermis compared to more hydrophobic scaffold variants.

To further study the differential expression of genes involved in wound repair and regeneration, we performed qRT-PCR on mRNA harvested from scaffold treated wounds 24 days post implantation. Within EG7 PTK-UR treated wounds, we observed a relative upregulation of genes associated with extracellular matrix proteins, cell adhesion complexes and remodeling enzymes required for the formation of robust and organized skin tissue. In addition to ECM related genes, we observed an upregulation of growth factors such as TGFB, SMAD3, FGF7 and PDGF in EG7 PTK treatments compared to 900t PE and EG1 and EG2 PTK-UR scaffolds indicating increased cell proliferation, vascularization and collagen remodeling.

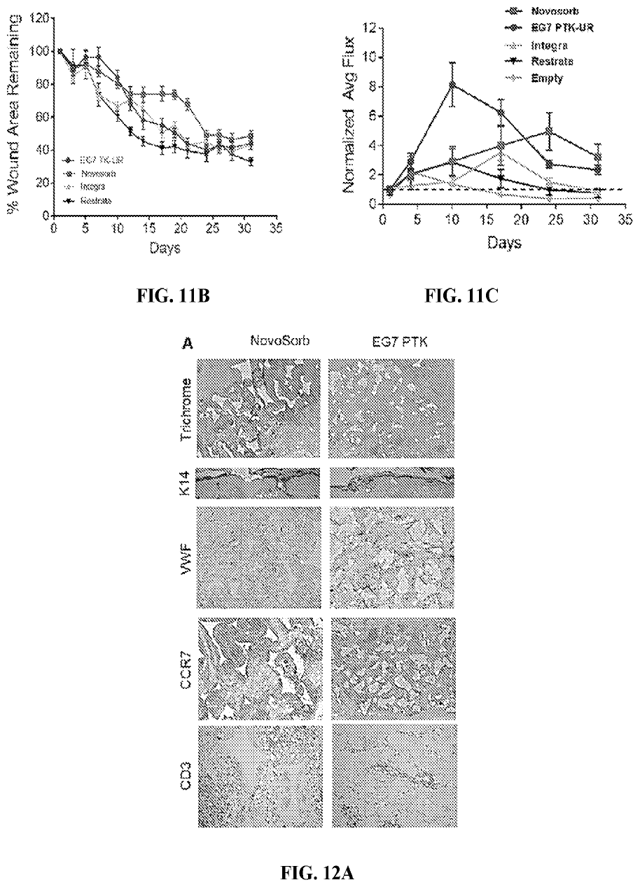

EG7 PTK-UR Treated Wounds Show Higher Blood Perfusion with Similar Rates of Wound Closure Compared to Clinically Approved Dermal Substitutes (FIGS. 11A-C).

We compared wound closure rates and blood perfusion of EG7 PTK-UR scaffolds to Integra Bilayer Wound Matrix, Restratra and NovoSorb, three clinically approved dermal substitutes. All treatments promoted wound healing over a period of 30 days.

Restrata provided minimal mechanical stenting to the wounds. EG7 PTK-UR and Novosorb scaffolds provided necessary wound stenting in the first 10 days of treatment. Adequate stenting is required for formation of granulation tissue and deposition of collagen to replace lost tissue. Bilayer architecture of Novosorb provided additional stenting of the wounds, however removal of the protective bonded top layer (as per manufacturer guidance) of Novosorb 3 weeks after application resulted in rapid decrease in wound area as well as re-wounding and bleeding of wounds, which could result in loss of new granulation tissue. Wound closure between Integra BMW and EG7 PTK-UR treated wounds were similar 30 days after treatment.

Laser doppler perfusion imaging (LDPI) of wounds showed significantly higher blood perfusion in EG7 PTK-UR treatments compared to other treatments up to 10 days post treatment demonstrating robust tissue integration and vascularization within the scaffold.

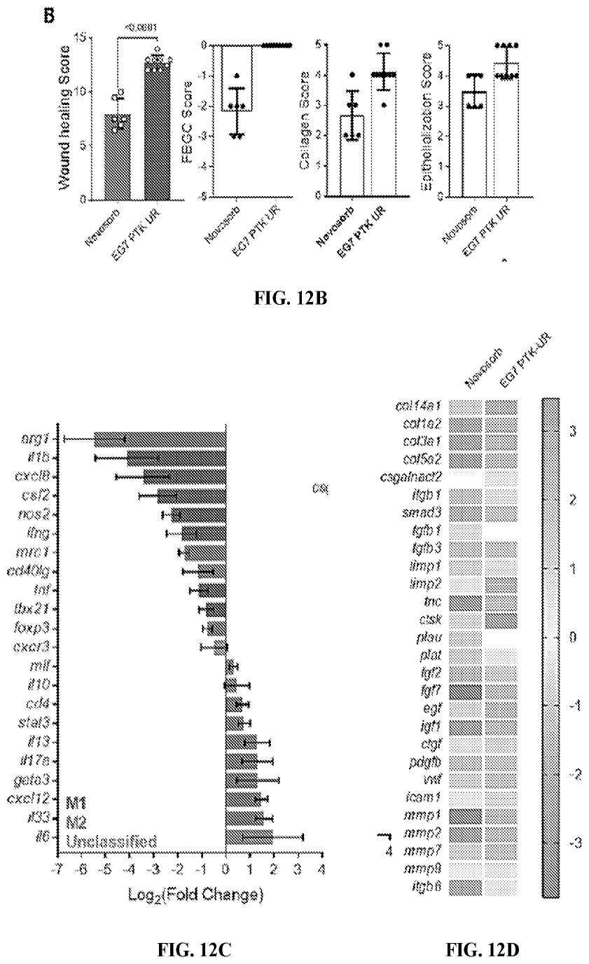

### Effective Immune Modulation and ECM Remodeling in PTK-UR Treated Wounds Result in Increased Quality of Wound Repair Compared to Clinically Approved Polyester-Based Dermal Matrix (FIGS. 12A-D).

We further investigated differences in tissue response towards EG7 poly(thioketal) and polyester based NovoSorb. Both scaffolds demonstrated robust infiltration and scaffold integration over a period of 30 days post implantation. However, Novosorb scaffold remnants were associated with highly cellular granulation tissue compared to PTK-UR samples suggesting persistent inflammation surrounding implanted scaffold (Trichrome). In addition to low cellularity and higher collagen deposition, a thin uniform epithelial layer was observed in wounds treated with EG7 PTK-UR scaffolds (CytoK14). In comparison, Novosorb treated wounds demonstrated thicker and hyperproliferative epithelium indicating persistent epidermal inflammation and poor keratinocyte migration. Increased vasculature was also observed in PTK scaffolds compared to NovoSorb (VWF). Moreover, EG7 PTK-UR scaffold pieces were associated with fewer CCR7+ proinflammatory macrophages as well as fewer CD3+ T cells compared to Novosorb suggesting decreased long-term inflammation associated with scaffold remnants.

Treatment blinded histopathological scoring of H&E and trichrome sections of scaffolds treated wounds showed an overall higher wound healing score of PTK-UR treated wounds including reduced foreign body giant cells score and higher collagen and epithelialization scores compared to Novosorb. In addition to histological assessment, gene expression profiling of wound tissue demonstrated overall downregulation of pro-inflammatory genes and upregulation of pro-healing type 2 genes in EG7 PTK treated wounds compared to Novosorb indicating higher resolution of inflammation within the wound bed. Additionally, EG7 PTK-UR treated wounds were characterized by upregulation of genes associated with ECM components and remodeling enzymes suggesting higher quality of wound repair compared to polyester based Novosorb treatment.

### Example 2

This Example describes how hydrophilic polythioketal urethane foam scaffolds promote skin wound healing in pigs. Porous, resorbable biomaterials can serve as temporary scaffolds that support cell infiltration, tissue formation, and remodeling of nonhealing skin wounds. Synthetic biomaterials are less expensive to manufacture than biologic dressings and can achieve a broader range of physiochemical properties, but opportunities remain to tailor these materials for ideal host immune and regenerative responses. Polyesters are a well-established class of synthetic biomaterials, however acidic degradation products released by their hydrolysis can cause poorly controlled autocatalytic degradation. Here, we systemically explored reactive oxygen species (ROS)-degradable polythioketal (PTK) urethane (UR) foams with varied hydrophilicity for skin wound healing. The most hydrophilic PTK-UR variant, with 7 ethylene glycol (EG7) repeats flanking each side of a thioketal bond, exhibited the highest ROS reactivity and promoted optimal tissue infiltration, extracellular matrix (ECM) deposition, and re-epithelialization in porcine skin wounds. EG7 induced lower foreign body response, greater recruitment of regenerative immune cell populations, and resolution of type 1 inflammation compared to more hydrophobic PTK-UR scaffolds. Porcine wounds treated with EG7 PTK-UR foams had greater ECM production, vascularization, and resolution of pro-inflammatory immune cells compared to polyester UR foam-based NovoSorb Biodegradable Temporizing Matrix (BTM)-treated wounds, and, and greater early vascular perfusion and similar wound resurfacing relative to clinical gold standard Integra Bilayer Wound Matrix (BWM). In a porcine ischemic flap excisional wound model, EG7 PTK-UR treatment led to higher wound healing scores driven by lower inflammation and higher re-epithelialization compared to NovoSorb BTM. PTK-UR foams warrant further investigation as synthetic biomaterials for wound healing applications.

### Introduction

Chronic wounds affect about 4.5 million people in the United States, resulting in considerable economic burden of $96.8 billion per year (1, 2). Diabetes, obesity, and vascular diseases predispose patients to delayed or incomplete skin wound closure that can lead to, lower limb amputation (1, 3, 4) and is about associated with high recurrence and mortality rates. (5, 6). Nonhealing wounds have persistent, low-grade inflammation with an influx of polymorphic mononuclear leukocytes (PMNs) that secrete inflammatory cytokines, proteolytic enzymes, and reactive oxygen species (ROS) (7). Nonhealing wounds driven by vascular insufficiencies are often compounded by ischemia-reperfusion injury which triggers ROS generation and tissue damage (8), while bacterial infection can further exacerbate the local inflammatory imbalance (10). Excess ROS causes oxidative stress which diminishes critical wound healing processes such as angiogenesis, extracellular matrix (ECM) deposition, and the healing capacity of fibroblasts and keratinocytes (9).

The standard of care for treating chronic wounds includes tissue debridement, moisture control, and use of advanced wound dressings with or without active compounds to reduce infection (11). Wound dressing W biomaterials comprise either naturally derived or synthetic polymers that act as a provisional matrix, providing mechanical support and promoting cellular infiltration, neovascularization, ECM deposition, and consequently accelerated defect repair (12). Acellular wound dressings based on animal-derived ECM components are well-established. The first of its kind, Integra Bilayer Wound Matrix (BWM), designed by Yannas et al. (13), has a long clinical track record; in diabetic foot ulcers, Integra BWM achieves full closure in 51% of wounds by 16 weeks, compared to 32% by standard wound care (14). Similarly, OASIS ULTRA wound dressings manufactured from porcine small intestinal submucosa (SIS) decrease time required for complete wound closure and lower reoccurrence of venous ulcers (15). ECM-based dressings have been adopted for clinical use (16), but they suffer from reliable raw material sourcing, reproducible processing techniques, cross-species immunogenicity risk, and exuberant costs (17).

Fully synthetic dermal materials provide to ECM-based dressings reproducible and cost-effective product fabrication, while also affording controlled tuning of chemical composition, microarchitecture, physiochemical properties, and degradation profiles. Hydrolytically degradable polyesters (PE) are the main class of synthetic polymers that have so far been applied clinically as resorbable wound dressings (21), with Restrata and NovoSorb Biodegradable Temporizing Matrix (BTM) being two examples. Restratais an electrospun poly (lactic acid-glycolic acid) (PLGA) (10:90) and polydioxanone mat, while NovoSorb is a polyurethane (PU) foam made from ethyl lysine diisocyanate, lactic acid/ethylene glycol chain extender, and polycaprolactone polyol with a removable polyurethane overlayer (22, 23). Both products have gained broad United States food and drug administration approval for indications spanning traumatic and post-surgical wounds, second degree burns, pressure ulcers, and diabetic ulcers.

Highly porous polyurethane foams, such as Novosorb, offer: interconnected pores that allow for efficient cell infiltration and low material mass, which reduces production costs as well the quantity of potentially inflammatory or cytotoxic degradation products. Polyurethane materials are also tunable, through variation of either the polyol or isocyanate-containing component. Isocyanates have been paired with aliphatic PE such as poly(lactic acid) (PLA) (25), PLGA, polycaprolactone (PCL) (26), and their blends for many tissue engineering applications (27, 28, 29, 30). However, degradation of PE based materials releases acidic byproducts which can activate an uncontrolled autocatalytic degradation mechanism (31) and trigger inflammation (32).

Hydrolytically stable, stimuli-responsive polymeric biomaterials that are degraded by cell-generated stimuli are a promising alternative to PE-based materials (33). We previously innovated a class of polythioketal (PTK)-based polyurethane scaffolds that provided more temporally controlled degradation kinetics compared to PE materials in rat wounds and promoted robust tissue integration both in rat and porcine skin wounds (34, 35). We have also shown that PTK crosslinked polyethylene glycol (PEG)-)maleimide hydrogels promote tissue infiltration and neovascularization in mice, confirming the biocompatibility of hydrophilic PTK-based materials in physiologically relevant environments (36).

Here, we posited that scaffold hydrophilicity is an important parameter for optimization of wound healing performance of PTK urethane (PTK-UR) dermal substitutes. This hypothesis was motivated by previous studies showing that integration of PEG can increase scaffold degradation (37) and increase tissue infiltration of hydrolytically degradable PE-UR scaffolds in vivo (25), and observations that hydrophilic polymers such as PEG and zwitterions can decrease fibrosis and trigger a regenerative immunophenotype (38). Toward this end, we generated a library of ROS-responsive PTK-UR foams with controlled variation of the PTK diol component to contain different lengths of ethylene glycol (EG) units between the thioketal (TK) bonds in the polymer backbone. We hypothesized that increased hydrophilicity of PTK-UR scaffolds would increase TK group reactivity with water soluble ROS, thus improving scaffold ROS scavenging and ultimately promoting a faster and more favorable rate of material resorption in vivo. The primary goals of these studies were to establish the functional importance of PTK-UR scaffold hydrophilicity on wound healing and to benchmark leading PTK-UR formulations against current standards of care in clinically relevant porcine skin wound repair models that recapitulate full-thickness and ischemic skin wounds.

### Results

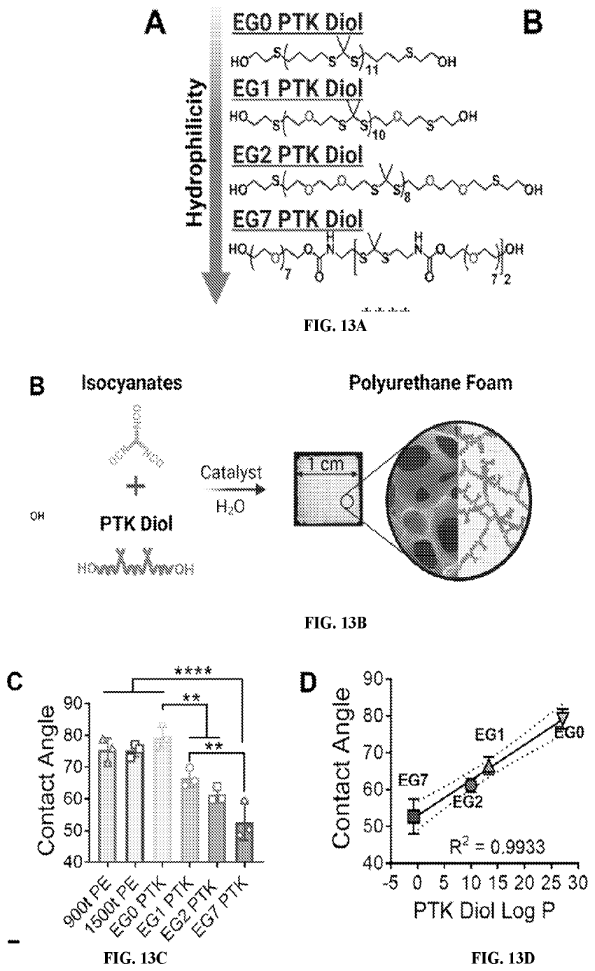

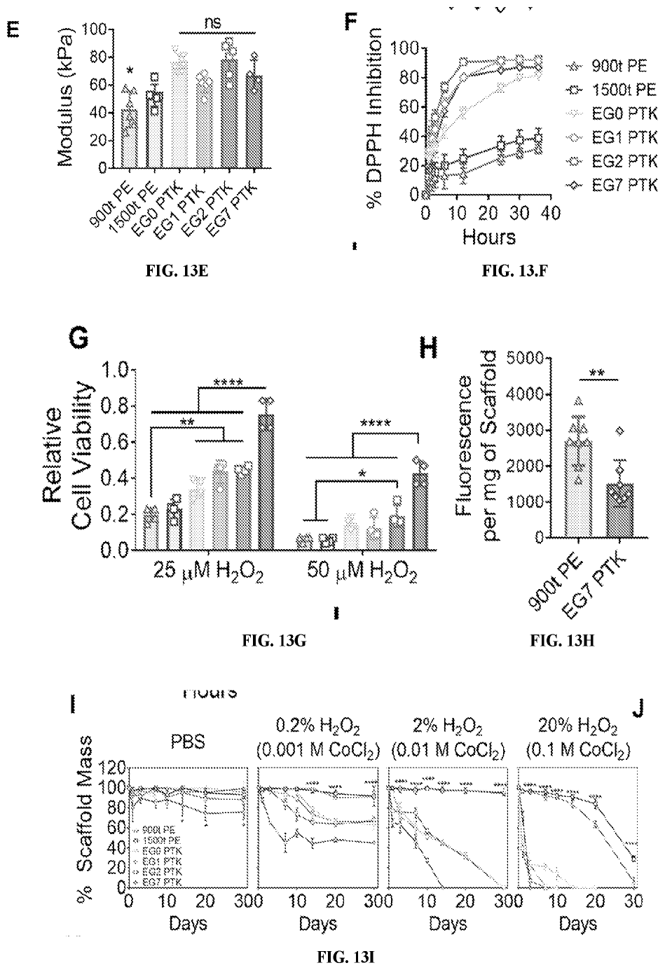

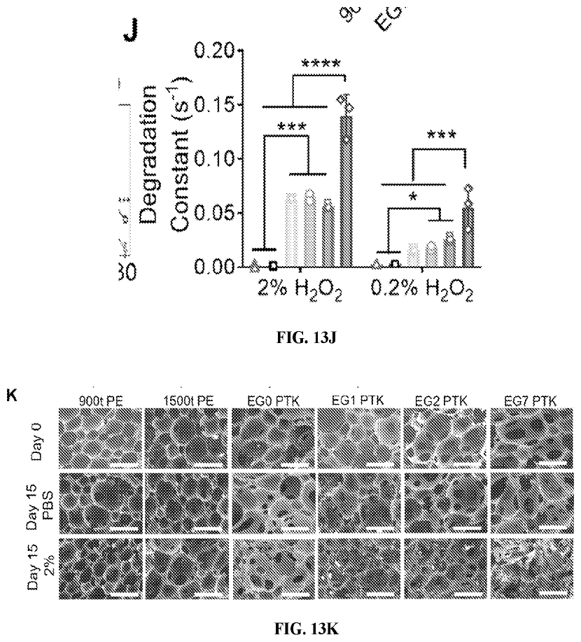

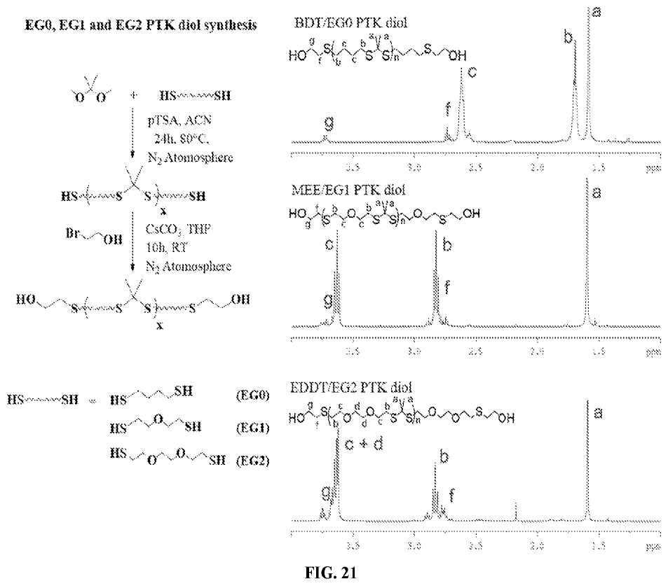

Synthesis and characterization of PTK diol To increase polyol hydrophilicity, we incorporated increasing units of EG between thioketal (TK) bonds in the PTK diol backbone using three different dithiol monomers with zero (EG0), one (EG1) or two (EG2) EG repeats. Using previously reported polymerization techniques (34, 35, 39, 40), we), we synthesized EG0, EG1, and EG2 PTK dithiols through condensation polymerization of butanedithiol (BDT), mercaptoethyl ether (MEE), and 2,2′-(ethylenedioxy) diethanethiol (EDDT) monomers, respectively (FIGS. 13A and 21). Purified dithiol polymers were effectively functionalized with 2-bromoethanol to obtain EG0, EG1, and EG2 PTK diols. The formation of TK bonds was confirmed through ¹H NMR; 6 at 1.6 ppm (FIG. 21). Dithiol monomers containing an EG content greater than 2 did not efficiently polymerize with this condensation reaction, resulting in poor conversion, likely forming cyclic structures from individual monomers rather than linear chains of multiple monomers.

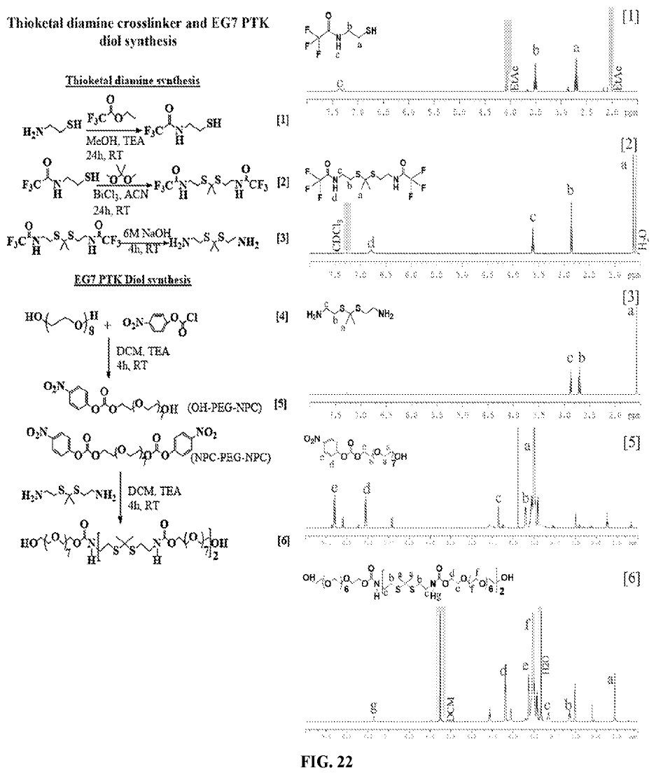

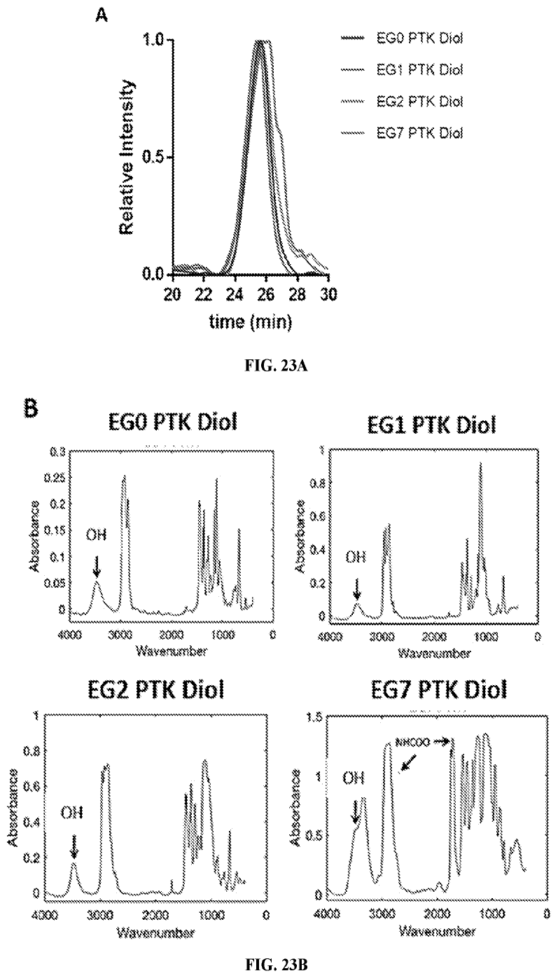

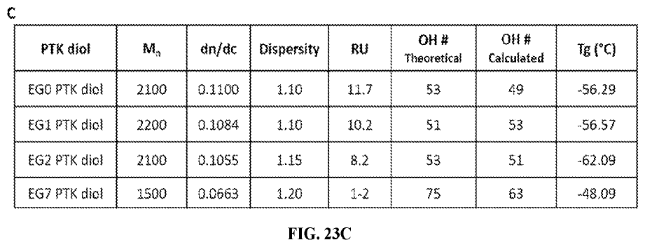

To address the drawbacks of this approach and to further increase the EG content, we developed a new, alternative polymerization strategy based on polyaddition reaction of amine reactive heterobifunctional PEG and a small thioketal diamine crosslinker/monomer. The thioketal diamine was synthesized from a protected cysteamine derivative and dimethoxy propane (DMP) as confirmed by ¹H NMR spectroscopy (FIG. 22) (41). To obtain PTK diol with seven EG repeats between TK groups in the polymer backbone (EG7 PTK diol), monofunctional PEG400 nitrophenylcarbonate (HO-PEG-NPC) was first synthesized through the reaction of 400 gmol⁻¹ PEG diol (PEG400) with p-nitrophenyl chloroformate at a stoichiometric ratio of 1:1 yielding a mixture of primarily OH-PEG-NPC and small percentage of homobifunctional PEG400 dinitrophenylcarbonate (NPC-PEG-NPC) macromers. The bifunctional PEG macromers functioned as “chain-extenders” through reaction with multiple thioketal diamines (FIG. 22). Gel permeation chromatography (GPC) analysis revealed that all PTK diol polymers chain had relatively low dispersity (Ð<1.2) and a number average molecular weight (Mₙ) of between 1500-22002 gmol⁻¹ (FIG. 23A). The resulting EG0, EG1, EG2, and EG7 PTK diols were composed respectively of 11.7, 10.2, 8.2, and 2 thioketal units per polymer chain (FIG. 23B), and the presence of terminal hydroxyl functional (OH) groups was verified by their characteristic FTIR absorbance peak at 3400 cm⁻¹. Hydroxyl functionalization was quantified indirectly through ¹⁹F NMR upon reaction with 4-fluorophenyl isocyanate. Experimentally determined hydroxyl numbers closely matched theoretical values indicating successful hydroxyl functionalization (˜85%) and presence of terminal OH groups for isocyanate reactivity in subsequent foaming reactions (FIG. 23C).

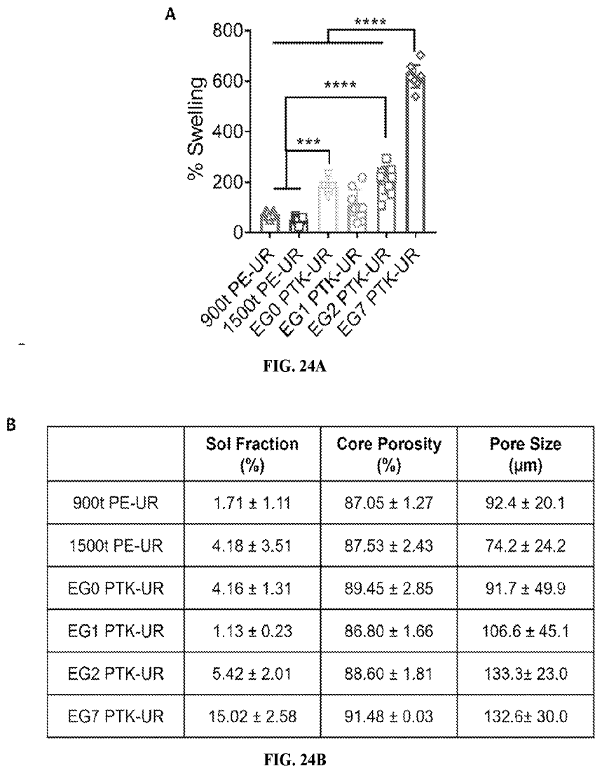

To create a polyurethane foam with interconnected porous structure, the terminal OH groups on PTK diols or PE trimeric polyols (trimer t, 60% E-caprolactone, 30% glycolide, and 10% D, L-lactide composition) were reacted with isocyanate groups present on a lysine triisocyanate (LTI) crosslinker in the presence of TEGOAMIN33 as a catalyst and water as a blowing agent (FIG. 13B). LTI was used based on our previous porcine ischemic wound healing studies which showed favorable response to PTK-UR scaffolds made with LTI as compared to scaffolds formulated using a non-degradable isocyanate component (35). Two PE-based triols with molecular weights 900 gmol⁻¹ (900t PE) and 1500 gmol⁻¹ (1500t PE) were used to fabricate PE-UR scaffold controls. Formulation specifications for each foam are provided in Table 1. PTK-UR scaffolds with similar porosity (86.8%-91.5%) and comparable pore sizes (74.2-133.3 μm) were fabricated from the respective EG0, EG1, EG2, and EG7 PTK diols. Efficient covalent crosslinking between OH and NCO-groups of LTI was confirmed based on the low soluble fractions (sol fractions) for each scaffold (FIGS. 24A-B).

TABLE 1

| | 900t | 1500t | EG0 | EG1 | EG2 | EG7 |
|---|---|---|---|---|---|---|
| | PE | PE | PTK | PTK | PTK | PTK |
| Polyol | 100 | 100 | 100 | 100 | 100 | 100 |
| Calcium stearate | 4 | 4 | 4 | 4 | 4 | 4 |
| TEGOAMIN33 | 2.3 | 2.3 | 2.3 | 2.3 | 2.3 | 2.3 |
| Water | 1.5 | 1.5 | 1.5 | 1.5 | 1.5 | 0.5 |
| Sulfated caster | 1 | 1 | 1.5 | 1.5 | 1.5 | 2 |
| oil | | | | | | |
| LTI | 51.8 | 38 | 27.6 | 27.6 | 27.6 | 23 |

Contact angle measurements on PU films were performed to experimentally gauge scaffold hydrophilicity; EG7 PTK-UR demonstrated the lowest contact angle (52.7±5.8°) compared to EG0 (79.2±3.3°), EG1 (66.5±2.9°), and EG2 (61.2±2.3°) (FIG. 13C). The measured contact angles positively correlated with the theoretical partition coefficients (Log P) of PTK diols (FIG. 13D). Determination of the swelling ratio for the PTK-UR materials (FIGS. 24A-B) also supported that our EG series yielded a set of PTK-UR scaffolds with controlled variation of relative hydrophilicity. Mechanical properties (compressive modulus determined under hydrated conditions) of the scaffolds were all similar with EG0, EG1, EG2, and EG7 PTK-UR scaffolds showing moduli of 75.0±7.8, 61.4±6.6, 78.5±12.3, and 67.1±10.7 kPa, respectively (FIG. 13E); these properties are a good approximation of the reported tensile modulus of human skin (60-850 kPa) (42, 43). While the wet modulus of all PTK-UR formulations did not significantly differ, the modulus of the 900t (P<0.05 versus all PTK-UR formulations) and 1500t PE-UR (1500t vs EG0, 1500t vs EG2, P<0.01) scaffolds showed a small but significant decrease compared to the PTK-URs. These data collectively indicate that the PTK-UR scaffold formulations have similar mechanical and structural properties, allowing for controlled study of the biological effects of relative scaffold hydrophilicity.

PTK-UR Scaffold Hydrophilicities Positively Correlate with EG Content and Log P Values

TK bonds are broken through irreversible reaction with ROS such as hydroxyl radicals (·OH), superoxide (O₂⁻), hydrogen peroxide (H₂O₂) and hypochlorite (ClO⁻) (34, 40, 44). This provides an opportunity to create TK-based biomaterials whose ROS-dependent degradation mechanism also provides antioxidant function. Using 1,1-diphenyl-2-picrylhydrazyl (DPPH) as a model free radical, the antioxidant activity of PTK-UR scaffolds was significantly higher than PE-UR scaffolds (P<0.0001 at 3, 6, 12, 24, 30, and 36 hours, FIG. 13F). The predominantly organic solvent mixture used to perform the DPPH assay (80:20 ethanol: H₂O) does not accurately reflect in vivo conditions, and experimental results may not reflect the differences in TK bond accessibility that occur in a fully aqueous system. Despite these conditions, EG1, EG2, and EG7 PTK-UR formulations showed significantly (P<0.001) higher radical scavenging compared to EG0 PTK-Urs (at 6, 12, and 24 hours) in this polar solvent mixture (FIG. 13F). To evaluate the effect of PTK composition on antioxidant function and cytoprotection in aqueous environments more directly, we exposed NIH 3T3 murine fibroblasts to varying concentrations of H₂O₂(25 μM and 50 μM) in the presence of the series of PTK diols (normalized thioketal molar content). All PTK diol treatments offered significant (P<0.05) protection from H₂O₂(25 μM)-mediated cell toxicity compared to 900t PE treatments. At the higher concentration of H₂O₂ (50 μM), EG7 PTK formulation rescued cell viability to a significantly (P<0.0001) higher degree than all other treatment groups, offering nearly 2-fold greater viability than the next most hydrophilic polythioketal (EG2 PTK) (FIG. 13G). These data highlight an important cytoprotective benefit of ROS-scavenging in the PTK diols and the correlation of PTK radical reactivity with hydrophilicity. To further evaluate the antioxidant activity of EG7 PTK-UR scaffolds, we implanted 900t PE-UR and EG7 PTK-UR foams in the subcutaneous space of C57BL6 mice. Scaffolds explanted 4 days post-implant showed that the EG7 PTK-UR foams had significantly (P<0.01) lower ROS concentrations than 900t PE-UR scaffolds measured by Amplex Red (an H₂O₂ probe) (FIG. 13H). These data suggest that presence of accessible thioketals in more hydrophilic polyurethane scaffolds provide antioxidant activity.

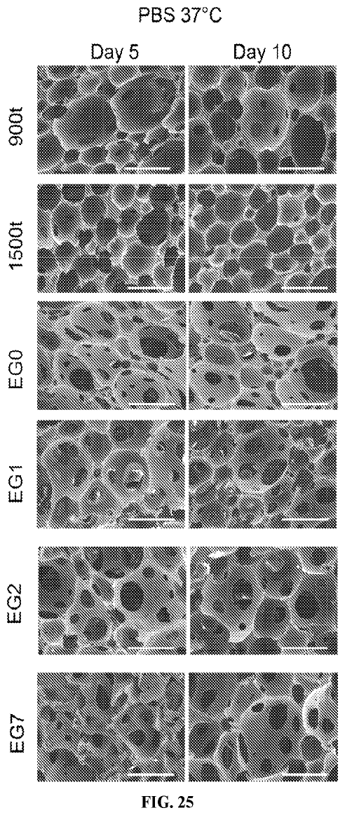

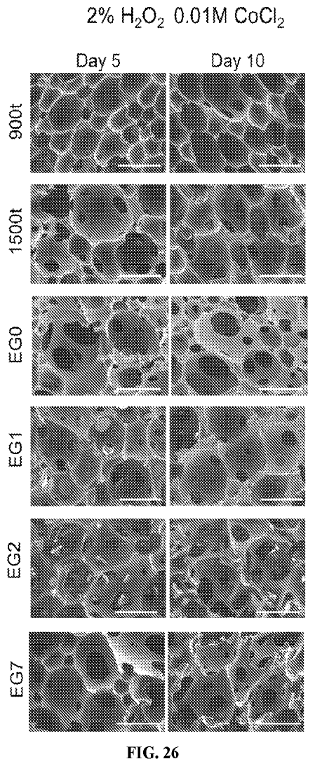

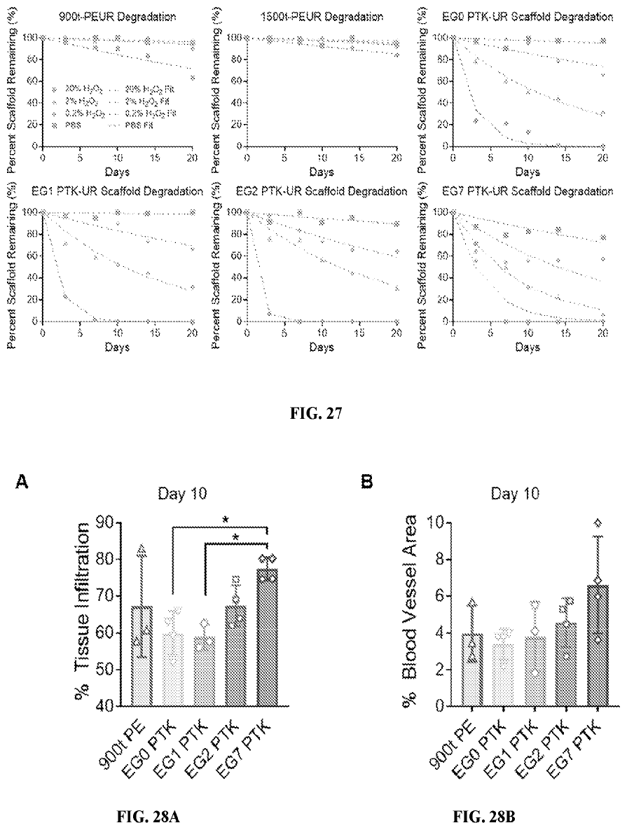

To investigate ROS-dependent degradation kinetics of PTK-UR and PE-UR scaffolds, we incubated PU samples in phosphate-buffered saline (PBS) or increasing concentrations of oxidative media (FIG. 13I). PTK-UR scaffolds incubated in hydrolytic media (PBS) remained intact with minimal mass loss over the course of the experiment. There was small but significant (P<0.05) mass loss of EG7 PTK-UR scaffolds in PBS at days 14, 20, and 30 due to the mechanics of manipulating the significantly (P<0.0001) more swollen EG7 PTK-UR scaffolds (FIG. 24A). However, unlike 900t and 1500t PE-UR scaffolds, all four PTK-UR foam compositions degraded in oxidative media (0.2% and 2% H₂O₂) simulated by ·OH from the Fenton reaction between H₂O₂ and catalytic amounts of CoCl₂. The PTK-UR scaffold mass loss was ROS concentration-dependent for all compositions. There were no significant differences in rate of degradation between the different PTK-UR scaffold formulations at the highest hydroxyl radical concentrations. However, at lower concentrations (0.2% and 2% H₂O₂), significantly (P<0.01) higher mass loss in EG7 PTK-UR scaffolds was recorded compared to EG0, EG1, and EG2 PTK-UR formulations, despite the EG7 PTK diol having the lowest TK bond backbone density contributing to oxidative degradation. Modeling analysis showed that the EG7 PTK-UR scaffolds had a higher degradation rate constant than all the PTK-UR formations at 0.2% and 2% ROS concentrations and that, under mild ROS conditions (0.2% H₂O₂), the rate of degradation increased with an increase in EG content (FIGS. 13J and 27). Morphological changes such as loss in porous architecture and qualitative increase in pore size were observed in scanning electron microscopy (SEM) images of PTK-UR scaffolds incubated in oxidative media (FIGS. 13K and 25-26). No apparent changes were visible in PBS-incubated samples, suggesting hydrolytic stability. Together with the DPPH and cytoprotection assays, these data demonstrate that thioketal groups in the more hydrophilic (higher EG content) scaffolds are more accessible to ROS scavenging.

### Effects of Scaffold Chemistry on Porcine Excisional Wound Healing

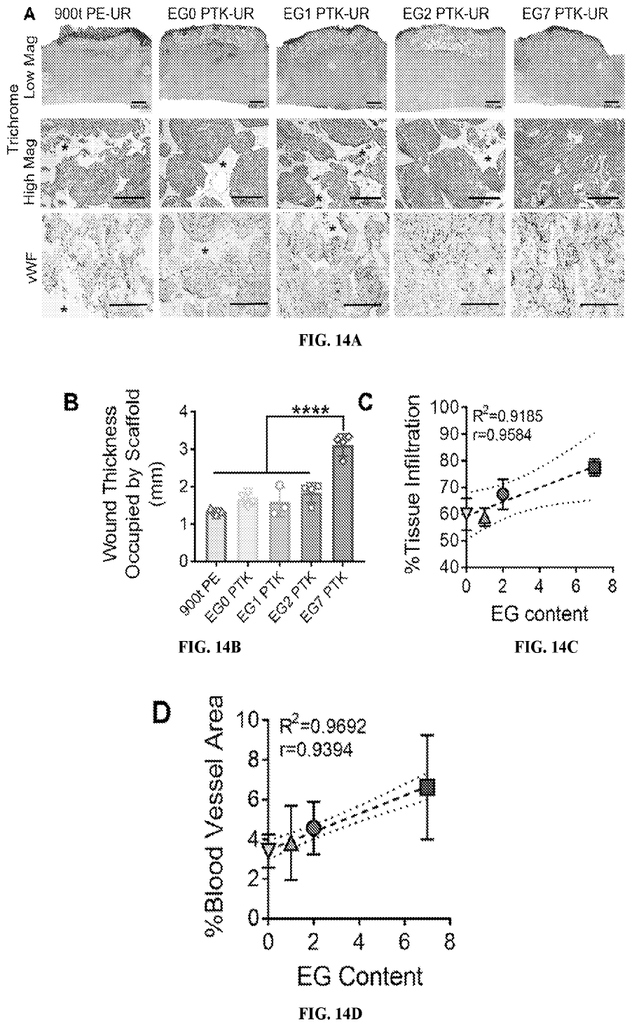

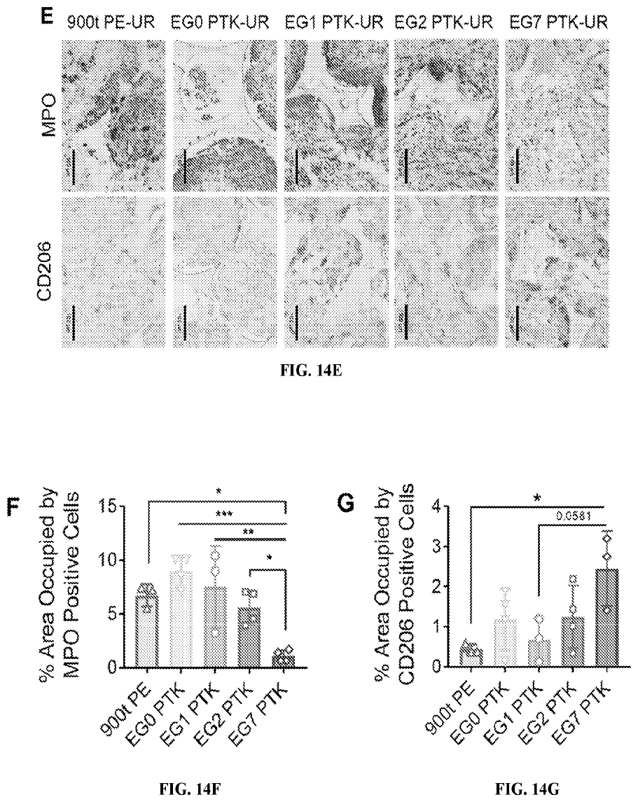

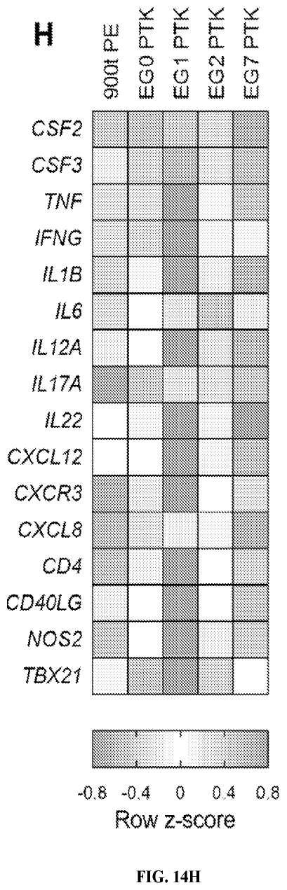

To investigate the tissue response to synthetic PU scaffolds, we implanted 900t PE-UR and PTK-UR scaffolds in rectangular 2 cm by 1 cm full thickness skin wounds on the dorsal region of Yorkshire pigs. 10 days after scaffold implantation, we observed varying degrees of scaffold integration within the wound bed (FIG. 14A, top row). An increase in tissue integration and homogeneity of distribution throughout the wound bed was observed for the wounds treated with EG7 PTK-UR compared to all other treatments (FIGS. 14A-B). Tissue infiltration within the pores of the scaffolds also positively correlated (R²=0.92) with EG content in the PTK diol used to fabricate the scaffolds (FIGS. 14C and 28A). The highest tissue infiltration was seen in EG7 PTK treatments (77%±3%) compared to the more hydrophobic PTK chemistries (FIG. 28B). Vascular density, visualized through immunohistochemistry (IHC) for von Willebrand factor (vWF), also positively correlated with EG content (FIGS. 14A, 14D, and 28B) and thus overall scaffold hydrophilicity.

### Scaffold Hydrophilicity Affects Inflammatory Cellular Infiltrates and Wound Microenvironment

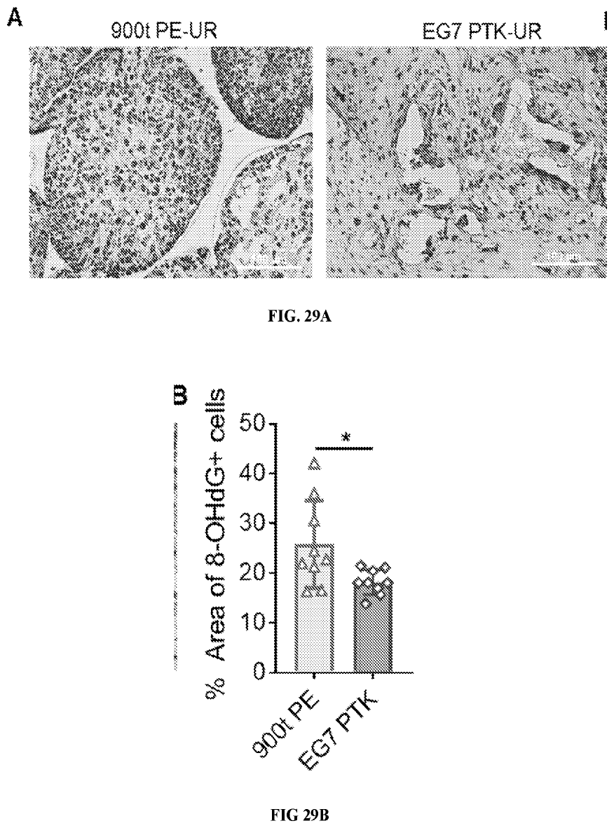

Inflammatory responses to implanted biomaterials can dictate the quality of tissue repair in skin wounds (45-47). We investigated spatial distribution of inflammatory cell infiltrates within implanted PU scaffolds 10 days post implantation. All five PU treatments recruited inflammatory cells, including myeloperoxidase (MPO)+ neutrophils and CD206+ macrophages, into the scaffolds (FIG. 14E). Active recruitment of neutrophils from the bloodstream to implanted biomaterials can propagate inflammation through secretion of pro-inflammatory cytokines, interleukins, and ROS (48, 49). MPO staining was localized around scaffold remnants, suggesting recruitment of neutrophils to the scaffold-tissue interface. EG7 PTK scaffolds were associated with significantly (P<0.05) fewer neutrophils (FIG. 14F) compared to all other treatments. A complementary immunohistochemical analysis for 8-hydroxy-2′-deoxyguanosine (8-OhdG), a marker for free radical-induced oxidative stress (50), showed significantly lower staining in EG7 PTK-UR-treated wounds compared to polyester based 900t PE-UR treatments (FIGS. 29A-B). Macrophage polarization can also play an important role in biomaterial integration and wound healing (45, 51, 52). We investigated the recruitment of M2 macrophages within implanted PU foams and saw a significant (P<0.05) increase in CD206⁺ macrophages in EG7 PTK-treated wounds compared to 900t PE treatment (FIGS. 14E and G). To complement histological analysis of wound tissue, we performed bulk tissue gene analysis of inflammation-associated transcripts; EG7 PTK-UR scaffold-treated wounds were associated with consistently decreased expression of inflammation-related mRNAs including colony stimulating factors (CSF2, CSF3), tumor necrosis factor-α (TNFA), interleukin 1β (ILIB), interleukin 12A (IL12A), inflammatory chemokine ligand 8 (CXCL8), and nitric oxide synthase 2 (NOS2) compared to all other treatment groups (FIG. 14H).

### EG7 PTK-UR Scaffolds Promote Wound Closure, Re-Epithelialization, and Tissue Repair in Pigs

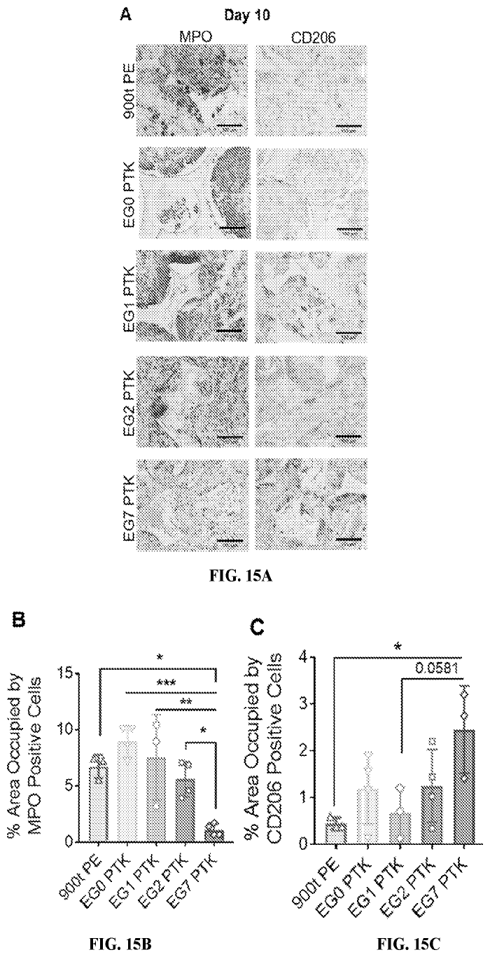

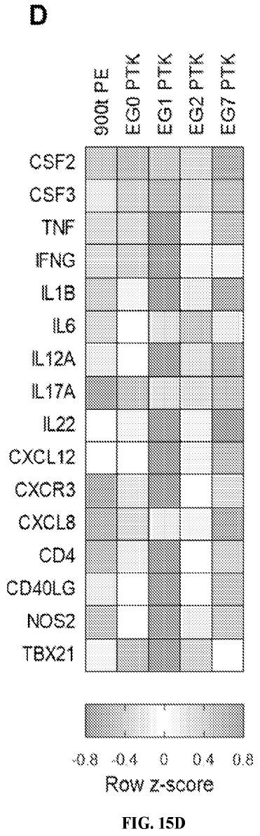

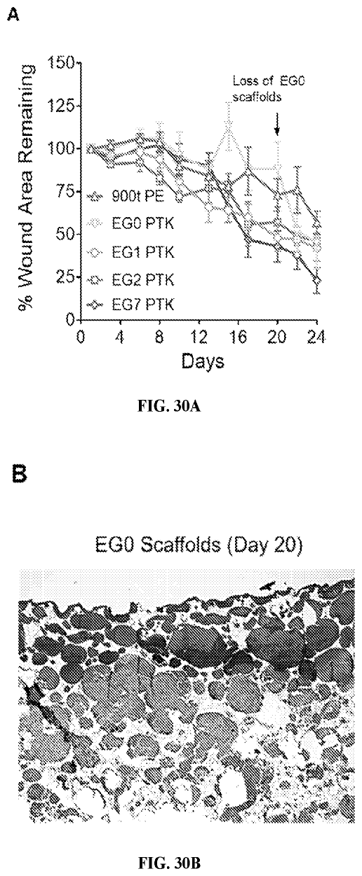

Next, we investigated wound closure rates of PU foam-treated pig skin wounds over an extended time course (24 days). Wound area for all scaffold treatments decreased over a period of 24 days (FIGS. 15A and 30A). 24 days after surgery, EG7 PTK-treated wounds achieved 70.5% closure compared to only 52.9% and 53.8% closure observed in EG1 and EG2 PTK treatments, as well as 49.2% for 900t PE (FIG. 15C). All EG0 PTK-UR scaffolds were extruded from the wounds 20-22 days post implantation, resulting in spontaneous wound contraction (FIG. 30A). We observed poor EG0 scaffold integration with skin wounds despite initially similar percentage of tissue infiltration between EG0 and EG1 scaffolds pores (FIGS. 28A and 30B) emphasizing the importance of scaffold hydrophilicity in biomaterial-tissue integration. Due to the premature extrusion and loss of EG0 scaffolds, this treatment group was not included in subsequent analyses.

Complete wound closure is associated with migration of epidermal cells from the surrounding wound margins towards the center, fully covering the granulation tissue with new epithelium. (53). We, therefore, sought to evaluate epidermal layer re-establishment of PU-treated excisional pig wounds through visualization of keratin14+ basal keratinocytes. Cytokeratin14 IHC revealed that at day 24, 90% of EG7 PTK-UR-treated wounds had completely epithelialized, with 99.2% of the wound covered with a thin, uniform neo-epidermal layer (FIGS. 15B-C). In contrast, none of the other PU treatments resulted in complete re-epithelialization of the wounds, with an average of 49.6%, 55.0%, and 61.0% wound closure observed in 900t PE, EG1, and EG2 PTK treatments, respectively (FIG. 15C). In addition to incomplete epithelialization of wounds with these treatments, excessive epidermal thickening suggests an immaturity of the re-epithelialization process due to persistent inflammation and presence of hyperproliferative keratinocytes with minimal migration (7, 54, 55).

Histological analysis of the skin wounds at the day 24 endpoint revealed tissue integration and scaffold resorption across all 4 PU treatments (FIG. 15D, low mag.). EG1 PTK and EG2 PTK scaffold remnants were primarily present in the lower portion of the granulation tissue, surrounded by a dense halo of immune cells. On the contrary, EG7 PTK-UR scaffolds were uniformly distributed within the granulation tissue and were not associated with dense pockets of inflammatory cells (FIG. 15D, high mag). To further investigate the effects of scaffold chemistry on different aspects of skin repair and restoration, trichrome and H&E sections were semi-quantitatively assessed by a treatment-blinded histopathologist (scoring rubric shown in Table 2). Scoring of the wounds indicated that EG7 PTK-UR-treated wounds had higher quality granulation tissue (FIG. 15E), more extensive ECM deposition and reconstruction of dermal architecture (FIG. 15F) compared to wounds treated with 900t PE. There was also an overall increase in quality and quantity of granulation tissue and collagen deposition associated with higher EG content, as indicated by granulation and collagen scores across the EG series. We also observed significantly (P<0.01) higher epithelialization of EG7 PTK-treated wounds compared to all other PU treatments (FIG. 15G). EG7 PTK-treated wounds were associated with lower inflammatory infiltrate, including mononuclear cells and foreign body giant cells (FBGCs), compared to 900t PE (FIG. 15H). The cumulative wound score revealed significantly (P<0.01) improved quality of wound healing and repair of EG7 PTK-treated wounds compared to more hydrophobic PE, EG1, and EG2 PTK formulations (11.5±1.34, 5.5±2.5, 6.9±2.7, and 7.33±2.1, respectively, FIG. 15I).

TABLE 2

| | | Granulation | | | |
|---|---|---|---|---|---|
| | | Tissue | | | |
| | Neovascularization | Maturation | Epithelialization | Inflammation | Collagen |
| 1 | Little evidence of | Little | Little migration | Mostly resolved | Little or no |
| | endothelial cell | evidence of | of epidermis | inflammatory | evidence of |
| | ingrowth into | mesenchymal | from wound | response. No | collagen |
| | wound site | cell | edges over | foreign body giant | fibers. |
| | | infiltration; | granulation | cells | Provisional |
| | | eschar | tissue | | matrix |
| | | predominant | | | consists of |
| | | | | | fibrin. |
| 2 | Angiogenic | Active | Initial | Residual | Initial signs |
| | response at the | expansion of | progression of | inflammatory | of collagen |
| | wound margins and | mesenchymal | epidermal | infiltrate with some | accumulation |
| | base; some | infiltration | resurfacing with | giant cells and | consisting of |
| | extravasation | from the base | less than 50% | mild mononuclear | very fine |
| | | and margins | coverage and | cell infiltrate | fibrils in a |
| | | of the wound, | hyperplasia | | random |
| | | accompanied | | | orientation. |
| | | by neutrophils | | | |
| | | and | | | |
| | | mononuclear | | | |
| | | cells | | | |
| 3 | >50% of | Continued | 50-90% coverage | Substantial | Moderate |
| | granulation tissue | filling of the | of wound site. | inflammatory | amount of |
| | shows | wound site | Clear polarized | infiltrate | collagen |
| | neovascularization | and | differentiation of | dominated by | deposition |
| | | displacement | epidermis | mononuclear cells | |
| | | of the eschar | | but significant | |
| | | above the | | persistence of | |
| | | wound | | polymorphonuclear | |
| | | surface | | cells, frequently | |
| | | | | near wound surface | |
| 4 | Extensive | Accumulation | 90-100% | Heavy | Loosely |
| | vascularization with | of | coverage of | inflammatory | assembled |
| | pruning and | extracellular | wound site with | infiltrate resulting | parallel |
| | maturation of | matrix, | initiation of rete | in impairment of | collagen |
| | neovessels | reduced | peg formation | healing | fibers |
| | | density of | and advanced | progression, and | |
| | | fibroblasts, | epidermal | invasion of | |
| | | and evidence | differentiation | adjacent | |
| | | of collagen | | unwounded tissue. | |
| | | fiber | | | |
| | | organization | | | |
| 5 | Advanced vascular | Extensive | Full keratinized | Wound site | Highly |
| | maturation with | reconstruction | epidermis with | dominated by | aligned |
| | thinning of capillary | of dermal | rete pegs and | inflammatory cells, | collagen |
| | networks | architecture | return of | including | fiber |
| | | with absence | epidermal | neutrophils, with | networks |
| | | of | hyperplasia to | consequent | with |
| | | inflammatory | thickness of | retardation of | tendency to |
| | | infiltrate | adjacent, | either granulation | form |
| | | | unwounded skin | tissue or epidermal | reticular |
| | | | | development. | arrays. |
| | | | | | Reduced |
| | | | | | fibroblast |
| | | | | | density. |

### Modulation of Innate and Adaptive Immune Response by EG7PTK-UR Scaffolds in Pig Skin Wounds

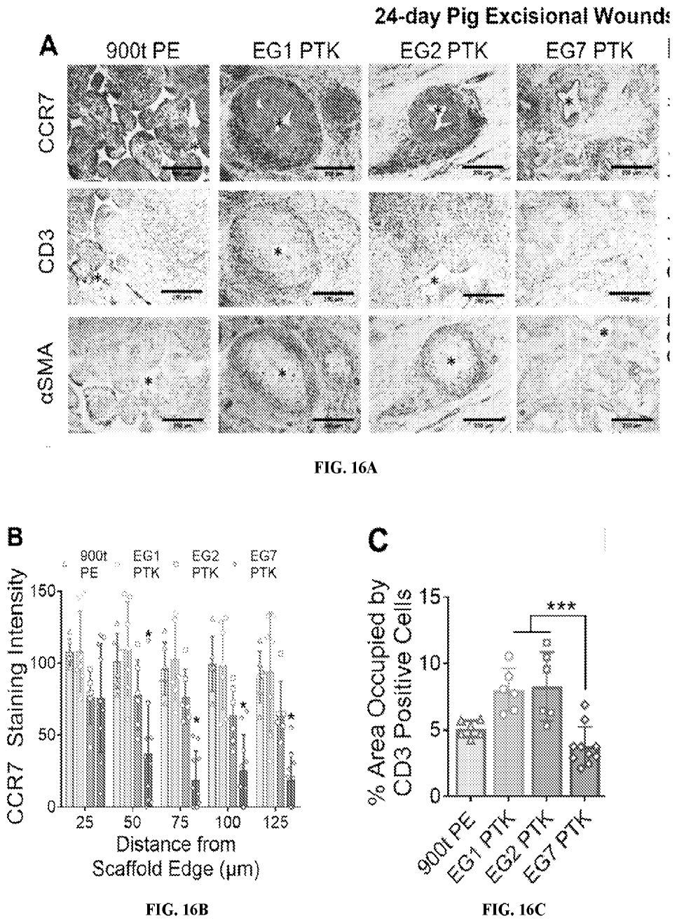

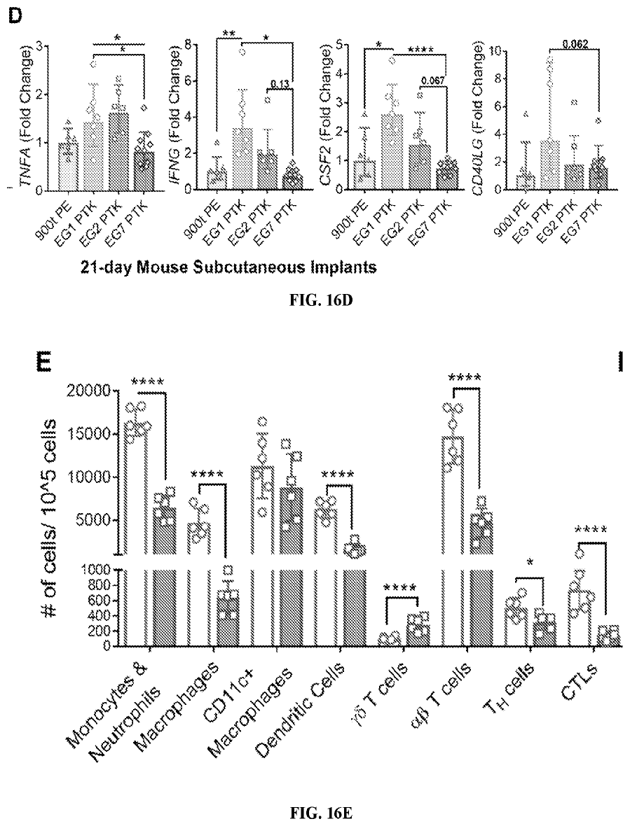

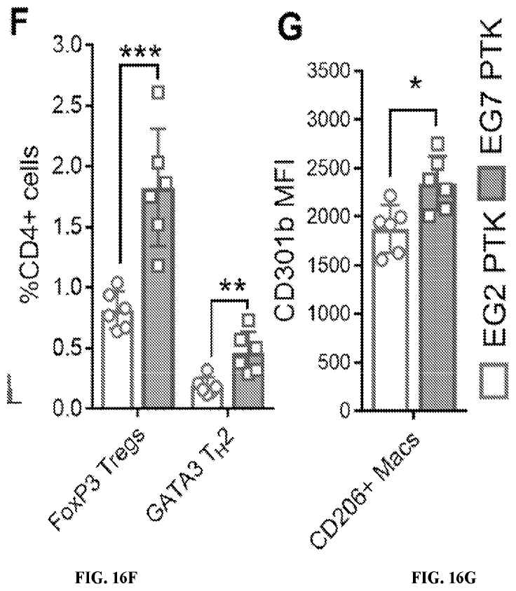

Inflammatory response to scaffold chemistry involving both innate and adaptive immune cells can impact biomaterial-tissue integration and wound repair (49, 56-58). We hypothesized that increased hydrophilicity of PU scaffolds contributes to decreased pro-inflammatory microenvironment within the wound, ultimately decreasing biomaterial-associated fibrosis. Accordingly, we evaluated the effects of scaffold chemistry on the recruitment of immune cells at the scaffold-tissue interface. 24 days after PU scaffold implantation, we confirmed the presence of both innate (CCR7⁺ M1 macrophages) and adaptive (CD3⁺ T cells) immune cells within the tissue margins of all implanted scaffold formulations (FIG. 16A). CCR7 IHC revealed a dense association of M1 macrophages with scaffold remnants, where CCR7 staining intensity decreased as a function of distance from the scaffold edge. There was also significantly (P<0.05) lower staining intensity surrounding EG7 PTK scaffold remnants compared to 900t PE, EG1, and EG2 PTK scaffolds at distances greater than 50 μm from scaffold-edge (FIG. 16B). Unlike EG7 PTK scaffolds, the other treatment groups were associated with a dense layer of M1 polarized macrophages and FBGCs surrounding scaffold remnants within the wound bed, suggesting a persistent inflammatory response to the scaffold and/or its degradation products. To evaluate the adaptive immune response, we also quantified the density of CD3 positive T cells surrounding scaffold pieces at day 24, finding significantly (P <0.001) lower density surrounding EG7 PTK scaffold remnants compared to EG1 and EG2 PTK scaffolds (FIG. 16C). T cells are known to produce IL6, IL12, and IL17 proinflammatory molecules in response to synthetic biomaterial implants, propagating inflammation, and fibrosis (59, 60). In addition to immune cells, we also looked at αSMA expression in myofibroblasts surrounding scaffolds. We observed structured organization of αSMA⁺ myofibroblasts surrounding EG1 PTK and EG2 PTK scaffold fragments but not EG7 PTK scaffolds (FIG. 16A). The concentric, spatial organization of M1 macrophages, myofibroblasts, and T cells suggests an active foreign body response against the more hydrophobic PTK-UR scaffold chemistries, similar to the response elicited by elicited by non-degradable implants (46, 61). These results suggest that the higher ROS degradability and scavenging potential of the more hydrophilic PTK-UR scaffolds is a critical factor in reducing the foreign body response to the biomaterials. It is also possible that the types of proteins that adsorb preferentially to hydrophilic versus hydrophobic material surfaces plays a role in downstream macrophage polarization (62-64) and ultimate healing response. To further evaluate the inflammatory microenvironment within PU scaffold treated wounds, we quantified bulk tissue expression amounts of proinflammatory genes (FIG. 16D). We observed significantly (P<0.05) lower expression of TNFA, a pro-inflammatory cytokine, in EG7 PTK treated wounds compared to EG1 and EG2 PTK treatments. Similar lower expressions were seen for proinflammatory mediators IFNG and CSF2.

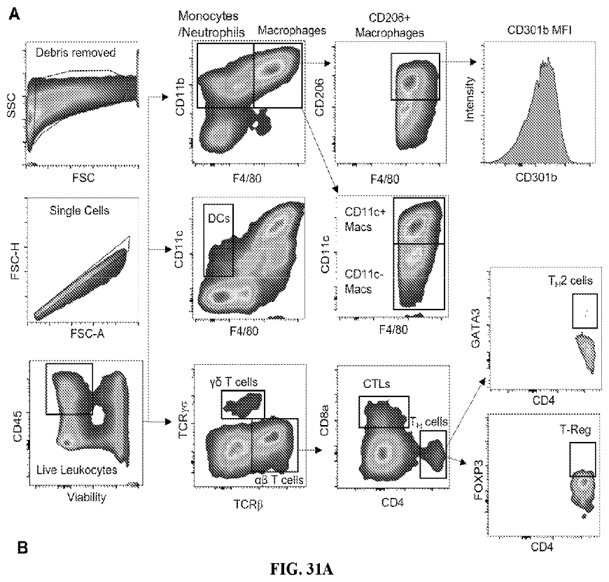

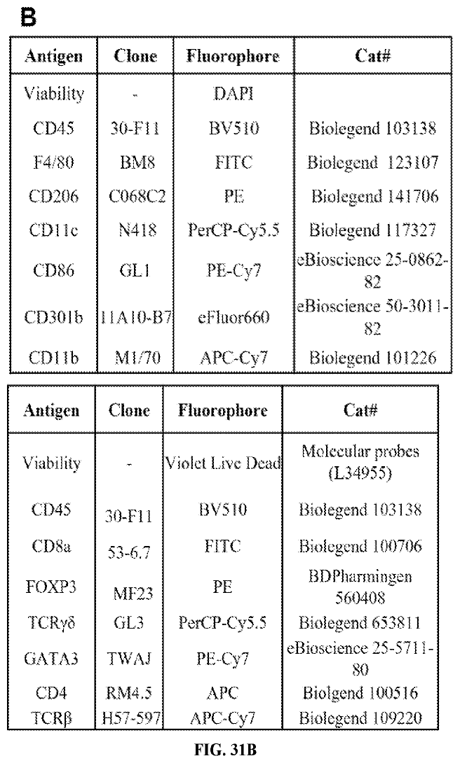

Due to loss in spatial resolution of inflammatory regions in bulk tissue gene expression analysis and limited availability of porcine specific antibodies for IHC, we utilized a mouse subcutaneous model to further characterize the immune cell populations and phenotypes that infiltrate the scaffolds. This flow cytometry study (gating strategies outlined in FIGS. 31A-B) focused on EG2 versus EG7 PTK-UR scaffolds, as these materials both effectively integrate in vivo while still showing significantly different hydrophilicity-driven biological responses. Scaffolds were implanted into ventral subcutaneous pockets in C57BL6 mice, and scaffold infiltrating cells were analyzed at 1, 2, and 3 weeks post-implantation. After 1 week, we observed similar proportions of CD45⁺leukocytes, CD11b⁺CD11c⁻F4/80⁺macrophages (20% of all leukocytes), CD11b⁺F4/80⁻ monocytes/neutrophils (˜40% of all leukocytes), and CD11c⁺F4/80⁻ dendritic cells (˜15% of all leukocytes) within the EG2 and EG7 scaffolds (FIGS. 32A-B). Over a period of 3 weeks, infiltrating leukocyte populations significantly (P<0.01) decreased in EG7 PTK scaffolds but remained constant in EG2 PTK scaffolds, suggesting an unresolved inflammatory response. We observed a significant (P<0.01) decrease in CD11b⁺CD11c⁻F4/80⁺macrophage population in EG7 PTK scaffolds compared to EG2 scaffolds. Decreases in CD11b⁺F480⁻ monocyte and neutrophil populations were also observed. After 3 weeks of scaffold implantation, we observed significantly (P<0.001) lower numbers of myeloid populations including CD11b⁺F4/80⁻ monocytes and neutrophils, CD11b⁺CD11c⁻F4/80+ macrophages, and CD11c⁺F4/80⁻ dendritic cells (DCs), as well as lymphoid-derived αβ⁺T cells, CD4⁺helper T cells (T_{H}), and CD8⁺cytotoxic T lymphocytes (CTLs) present in EG7 PTK cellular infiltrates compared to EG2 PTK (FIG. 16E). On further analysis of T cell phenotypes, we saw a significantly higher percentage of CD4⁺FoxP3⁺regulatory T cells (T-regs) (P<0.001) and CD4⁺GATA3⁺type-2 helper T cells (T_{H}2) (P<0.01) within EG7 PTK scaffolds compared to EG2 PTK-UR scaffolds (FIG. 16F). We also detected a higher number of γδ T (P<0.001) cells associated with EG7 PTK scaffolds; These cells are a source of fibroblast growth factor 9 (FGF9) and keratinocyte growth factor (KGF), which are both known to support re-epithelialization and hair follicle neogenesis after wounding (65, 66). We observed significantly (P<0.05) higher expression of CD301b, a macrophage galactose-type C-type lectin 2 (Mgl2) on CD206⁺macrophages isolated from EG7 PTK compared to EG2 PTK scaffolds (FIG. 16G). CD206⁺CD301b⁺macrophage subtypes support effective skin repair through the production of platelet derived growth factors (PDGF), transforming growth factor (TGFβ), and insulin like growth factor (IGF), resulting in fibroblast proliferation, migration, and collagen accumulation (67-69).

### EG7 PTK-UR Dermal Substitute Enables Effective Tissue Repair

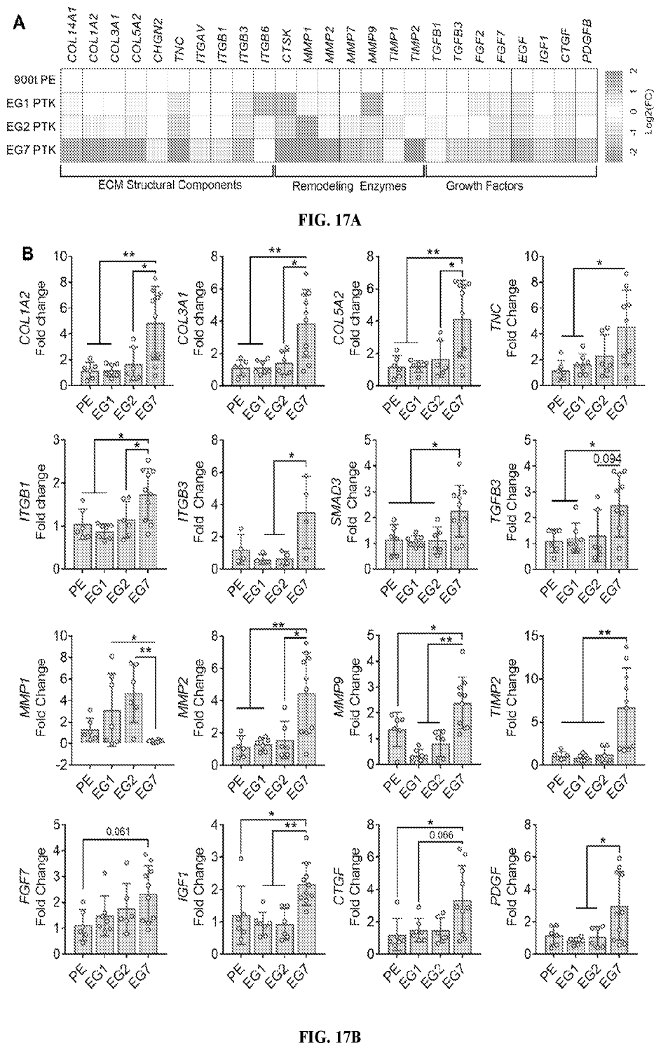

In addition to analysis of immune response, we measured the effects of scaffold chemistry on the expression of genes related to granulation tissue formation, proliferation, and remodeling processes of wound healing in day 24 pig wounds (FIGS. 17A-B). We observed that EG7 PTK scaffold-treated wounds had about 4-fold higher expression of genes encoding ECM proteins that comprise granulation tissue, including collagen I, III, V, and XIV (COL1A2, COL3A1, COL5A2, COL14A1) and tenascin (TNC). Integrins such as integrin β₁ (ITGB1) (P<0.05), integrin β₃ (ITGB3) (P<0.05), and integrin αᵥ (ITGAV) (P<0.05), known to play important roles in cell adhesion, migration, and proliferation (70, 71) were also significantly up regulated in EG7 PTK treatments. Transforming growth factor β (TGF-β) pathway-related genes such as SMAD3 (P<0.05) and TGFB3 (P<0.05), known to promote collagen biogenesis and polarization of macrophages to M2 pro-healing phenotype (72), were also significantly upregulated in EG7 PTK-treated wounds compared to other treatments. Matrix metalloproteinases (MMP2/7/9), which are known to play important roles in collagen remodeling and recruitment of endothelial cells for neovascularization (73, 74), were significantly (P<0.05) upregulated in response to EG7 PTK treatments with respect to other PE and PTK treatments. EG7 samples expressed lower amounts of MMP1, a type 1 collagenase upregulated in chronic wounds (75), relative to EG1 and EG2 PTK treatments. We also observed an increase in TIMP2 (tissue inhibitor of metalloproteinase), an important inhibitor of MMPs produced by basal keratinocytes, in EG7 PTK-treated wounds. Combined with the MMP expression data, this suggests that a homeostatic balance between MMPs and their inhibitors was achieved, reflective of active remodeling of wounds in a more mature stage of healing for the EG7 treated group (76). Similar trends of increased gene expression were observed in EG7 samples for genes such as insulin like growth factor (IGF), fibroblast growth factor 7 (FGF7), connective tissue growth factor (CTGF), and platelet derived growth factor (PDGF), soluble mediators that promote cell proliferation and migration to drive skin wound granulation, vascularization, and re-epithelialization (77).

### Benchmarking EG7 PTK-UR Against Clinically Approved Dermal Substitutes

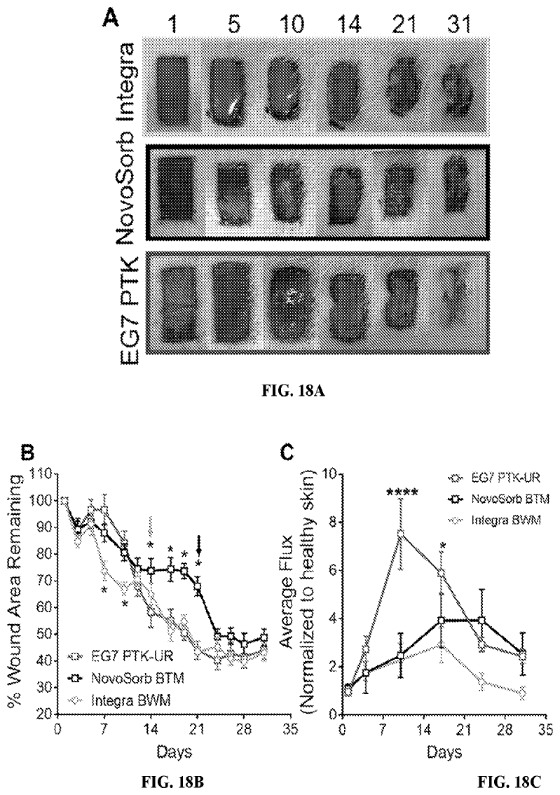

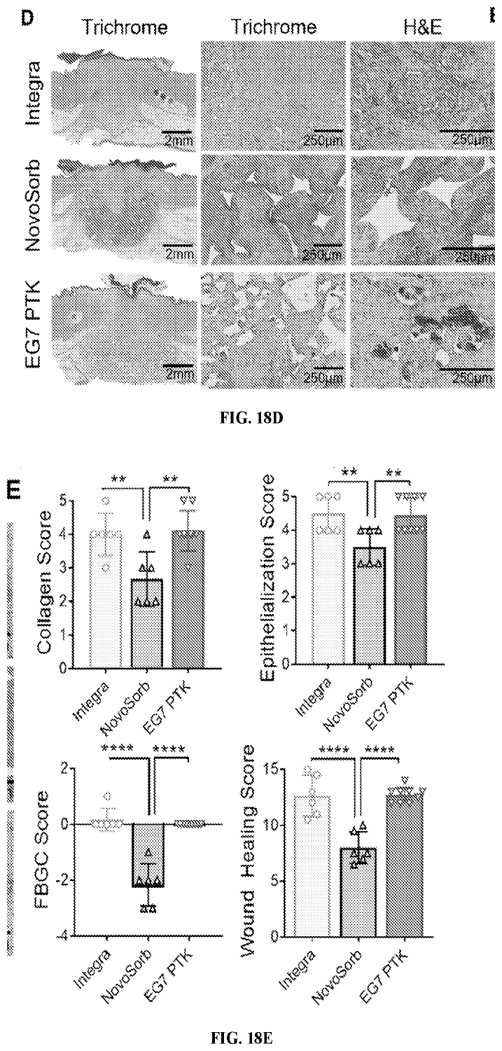

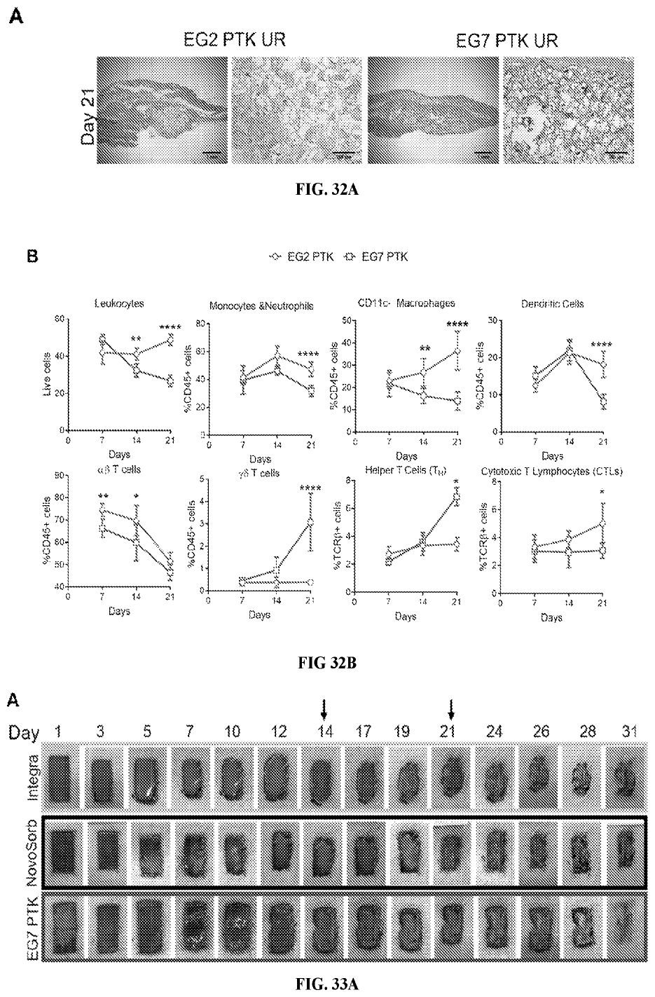

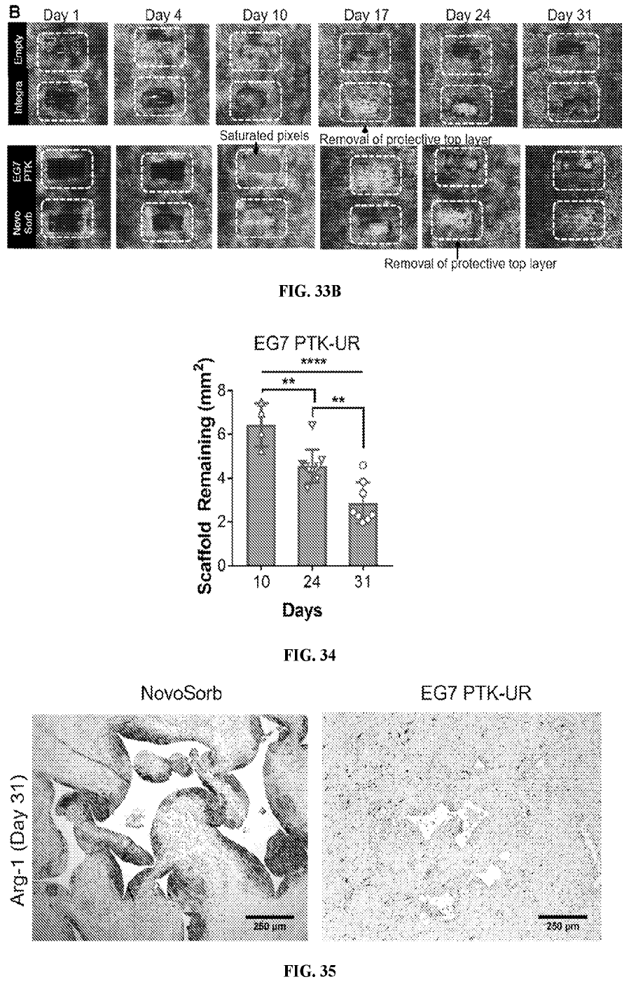

Performance of the EG7 PTK-UR scaffolds was tested in pig skin wounds in comparison to clinically approved dermal substitutes: collagen and proteoglycan-based Integra BWM (Integra Life Sciences), and PE-UR polyurethane NovoSorb BTM (PolyNovo). We implanted porcine full thickness skin wounds with EG7 PTK, NovoSorb, or Integra and measured wound closure over time as well as histological outcomes at 31 days post-implantation. All 3 dermal substitutes facilitated tissue infiltration and wound closure over a period of 31 days (FIGS. 18A and 33A). Integra provided significantly less wound stenting than EG7 PTK scaffolds for the first 10 days, while the commercial foam Novosorb and EG7 PTK performed similarly in this metric. In later stages, EG7 PTK-treated wounds resolved at a similar rate to Integra with no significant differences in the rate of wound closure. However, we observed significantly (P<0.05) increased rates of wound closure in EG7 PTK and Integra vs NovoSorb at days 14, 17, 19, and 21 post-implantation. Upon removal of the non-degradable polyurethane layer (performed according to manufacturer's guidelines), the wound area of NovoSorb-treated wounds significantly decreased (P<0.05), suggesting rapid loss of stenting provided by the protective layer, followed by consequent wound contraction (FIG. 18B). In addition to wound closure, we measured vascular perfusion of scaffold-treated wounds using laser doppler perfusion imaging (LDPI) (FIG. 33B). EG7 PTK-UR-treated wounds were significantly (P<0.0001) more perfused than Integra and NovoSorb at earlier time points (day 10 and day 17), suggesting higher neovascularization (FIG. 18C). Removal of the temporary epidermal layer equivalent membranes from Integra and NovoSorb (FIG. 18B, indicated by arrows) resulted in disruption of granulation tissue, causing re-wounding and increased perfusion within the wounds at days 17 and 24 respectively (FIG. 33B).

In all 3 treatment groups, histological examination of scaffold-treated wounds revealed formation of granulation tissue along with the re-establishment of an epidermal layer. Trichrome and H&E-stained sections revealed dense cellularity around NovoSorb scaffold remnants along with the formation of FBGCs that were minimally present or absent in EG7 PTK-treated wounds (FIG. 18D). EG7 PTK-UR-treated wounds were composed of denser, more organized collagen deposition, along with decreased cellularity around scaffold remnants. These observations indicate that the EG7 PTK-UR-treated wounds were more mature and had transitioned into a more advanced remodeling phase relative to Novosorb-treated wounds. Treatment-blinded histopathological scoring of these sections revealed similar collagen deposition, re-epithelialization, and FBGC density between Integra and EG7 PTK treated wounds (FIG. 18E). In contrast, NovoSorb-treated skin wounds showed significantly (P<0.01) lower collagen deposition with loosely arranged fibers, incomplete re-epithelialization, more persistent inflammatory infiltrate, and formation of FBGCs. Cumulative wound healing scores revealed significantly (P<0.0001) decreased quality of wound healing of NovoSorb-treated wounds compared to Integra and EG7 PTK-UR treatments, with no significant differences detected between the latter two materials. The collective EG7 PTK-UR histology from all time points was also aggregated and analyzed for in vivo scaffold degradation over time, showing that EG7 PTK-UR scaffolds underwent relatively constant resorption over time that is still ongoing 31 days after implantation (FIG. 34).

### Effective Dermal Repair and Immune Modulation by EG7 PTK-UR Treatments Compared to NovoSorb

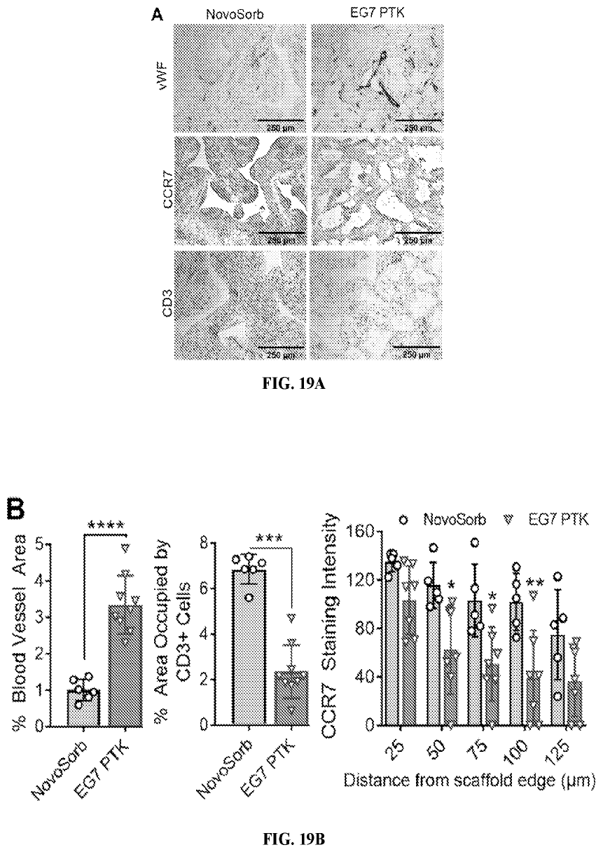

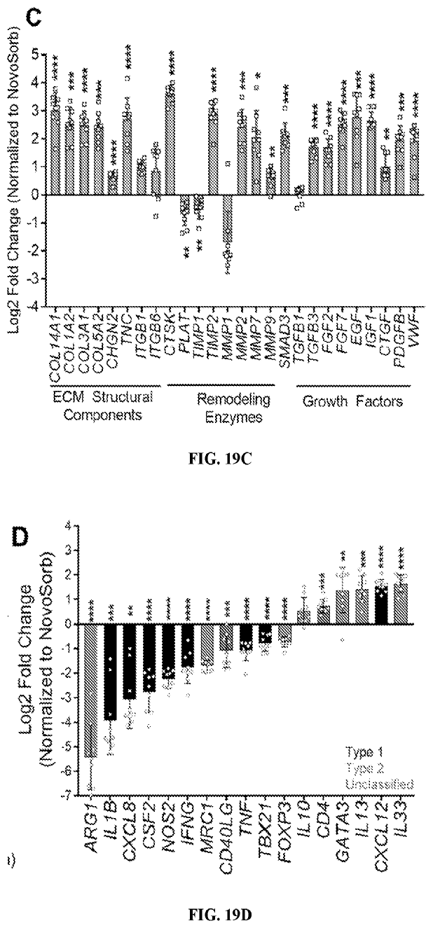

Next, we thoroughly characterized the microenvironment of EG7 PTK-UR scaffold-treated pig wounds versus the more structurally analogous, synthetic Novosorb PU foam-treated wounds. To complement the LDPI data, we measured vasculature within scaffold-treated wounds using vWF IHC and observed significantly (P<0.0001) higher blood vessel density within EG7 PTK-treated wounds compared to NovoSorb (FIGS. 19A-B). To further examine the differences in dermal repair and remodeling between the two foam dermal substitutes, we analyzed expression of genes involved in ECM deposition and remodeling along with growth factors implicated in dermal wound healing. Transcriptional analysis revealed significantly (P<0.05) higher expression of COL1A2, COL3A1, COL5A2, COL14A1, and TNC in EG7 PTK-treated wounds compared to NovoSorb (FIG. 19C). Additionally, significantly (P<0.05) higher expression of ECM remodeling enzymes including cathepsin K (CTSK), MMP2, MMP7, and TIMP2 were seen in EG7 PTK-treated wounds compared to NovoSorb. Factors involved in collagen biosynthesis and ECM remodeling such as TGFB3 and SMAD3 were also significantly (P<0.05) higher in EG7 PTK-treated wounds. The expression of mRNA encoding various growth factors including FGF2, FGF7, epidermal growth factor (EGF), IGF1, CTGF, and PDGF, implicated in endothelial, fibroblast, and keratinocyte proliferation and migration (77), were significantly higher in EG7 PTK-treated wounds compared to NovoSorb at day 31 post treatment.

Deeper analysis of innate and adaptive immune cells within scaffold-infiltrating tissue showed significantly (P<0.001) lower density of CD3 positive T cells in EG7 PTK-treated wounds compared to NovoSorb, suggesting resolution of the inflammatory wound healing phase along with decreased inflammation surrounding scaffold remnants (FIGS. 19A-B). Concurrently, we observed significantly lower density of CCR7 positive macrophages and FBGCs associated with PTK-UR scaffold remnants compared to NovoSorb, with CCR7 staining intensity varying as a function of distance from scaffold edge (FIGS. 19A-B). To further characterize the inflammatory response, we performed qRT-PCR on bulk wound tissue samples. Relative to NovoSorb, EG7 PTK-UR samples were associated with significantly (P<0.05) lower expression of pro-inflammatory genes such as interleukin 10 (ILIB) and TNFA. A similar decrease in expression of CXCL8 and CSF2, as well as M1 macrophage marker NOS2, M2 macrophage marker arginase-1 (FIG. 35), and T_{H}1 specific transcription factor T-bet (TBX21) were observed within EG7 PTK-treated tissue compared to NovoSorb. Decreases in pro-inflammatory markers in EG7 PTK-treated wounds were accompanied by a concomitant upregulation of anti-inflammatory markers such as T_{H}2 transcription factor GATA3 and interleukins including IL10, IL13, and IL33 that have been implicated in type 2 immunity, tissue regeneration, and wound healing (78-82).

### Enhanced Wound Closure, Blood Perfusion and Re-Epithelization of EG7 PTK-UR-Treated Porcine Ischemic Wounds

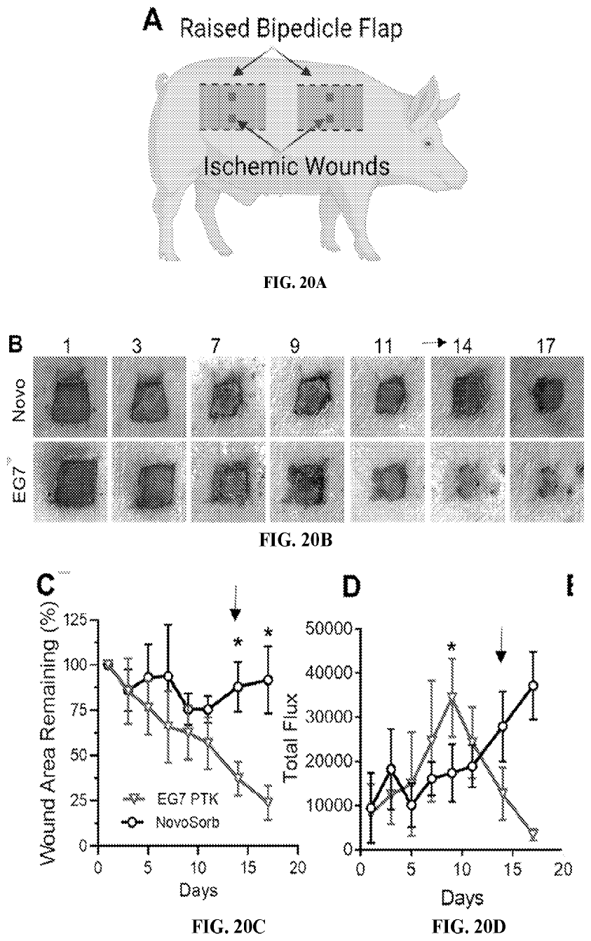

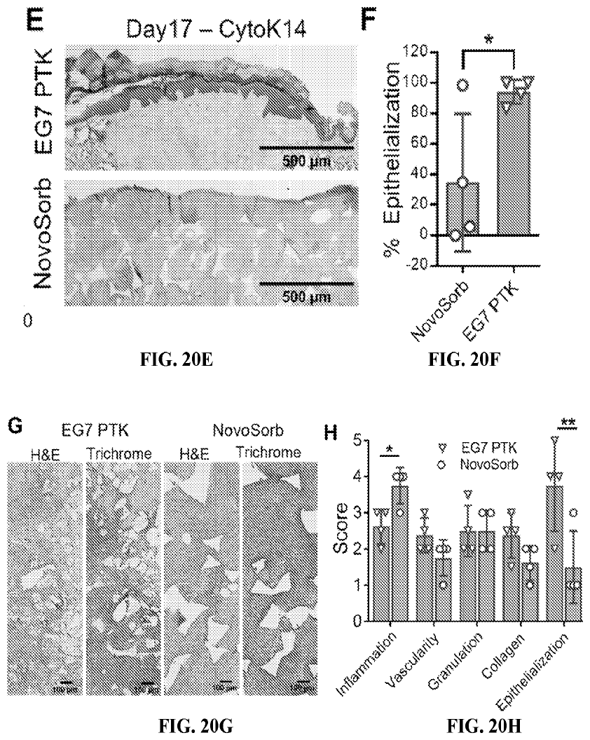

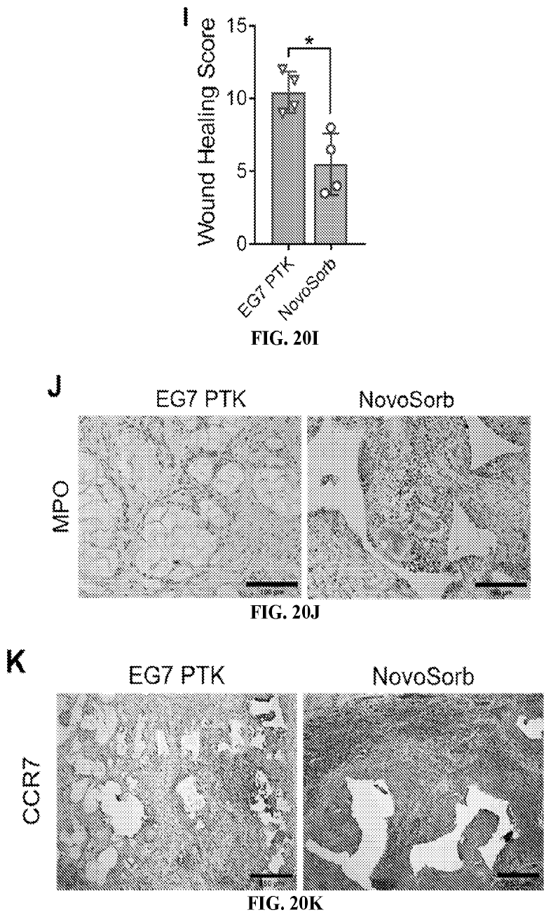

Ischemic wounds, such as those that can occur with venous leg ulcers, are associated with poor vasculature and delayed healing. To recapitulate slow-healing ischemic wounds in a porcine model, we used surgical cautery to create four raised bipedicle flaps in the dorsal region. Two 1 cm×1 cm excisional wounds were created in each of these flaps, which are separated from vascular sources from both the underlying muscle and the skin in either lateral direction (FIG. 20A) (35). We implanted these wounds with either EG7 PTK-UR or NovoSorb dermal matrices, and both wound closure (FIGS. 20 B-C) and blood reperfusion (FIG. 20D) were measured over a period of 17 days. Wounds treated with EG7 PTK-UR scaffolds healed quickly, showing 76.1% closure over 17 days, compared to 8.3% for NovoSorb. In addition to higher rate of wound closure, blood perfusion within EG7 PTK-UR-treated wounds was significantly higher (P<0.05) at day 9 post surgery with gradual return to the perfusion flux values of unwounded within the ischemic flap (FIG. 20D). Similar to non-ischemic wounds treated with NovoSorb, removal of the protective top-layer disrupted wound bed granulation tissue, causing re-injury and stimulation of a secondary vascular response. At the study endpoint (day 17), EG7 PTK-treated wounds were significantly (P<0.05) more re-epithelialized as visualized and measured using CytoK14 IHC (FIGS. 20E-F). Examination of H&E and trichrome-stained wound sections revealed significantly (P<0.05) increased cellularity and inflammation surrounding NovoSorb scaffold remnants compared to EG7 PTK-UR scaffolds (FIG. 20G). Histopathological scoring revealed that EG7 PTK-UR showed higher epithelialization, lower inflammation, and higher overall wound healing score relative to NovoSorb BTM-treated wounds (FIGS. 20H-I). Increased inflammation within the granulation tissue of NovoSorb-treated wounds was visualized through MPO (FIG. 20J) and CCR7 (FIG. 20K) IHC. NovoSorb scaffold remnants were surrounded by MPO+ neutrophils, suggesting more persistent inflammation within these wounds, whereas EG7 PTK-UR showed few residual neutrophils at day 17. Similarly, CCR7⁺macrophages and FBGCs were observed at higher density in NovoSorb-treated wounds compared to EG7 PTK-UR. These data support that EG7 PTK-UR scaffolds are promising for treatment of skin wounds hindered by lack of vascular supply and provide consistent improvement over the benchmark NovoSorb BTM in multiple wound healing scenarios.

### Discussion

Chronic skin wounds are growing in prevalence and have a 40% chance of recurrence (3), emphasizing the need for low cost and effective biodegradable dermal substitutes. Dermal substitutes provide immediate wound coverage without associated donor site morbidity, which is a major limitation for use of split thickness autologous skin grafts. Application of cell-based dermal substitutes such as Apligraf to treat venous leg ulcers decreases median time to complete wound closure compared to standard of care (83), but use of a living cellular component decreases the shelf-life and increases production costs (84, 85). Animal-derived acellular scaffolds like Integra BWM and Oasis ULTRA Tri-Layer Matrix can be stored at room temperature for long periods of time and have been successfully used to treat hard to heal wounds (14, 15). Integra BWM was initially indicated to treat burns in 2002 and has since been broadened for use in a variety of wound types. However, high cost [about $3855 per 4×5 inch graft (129 cm²)] financially burdens the healthcare system and the patient. Complications such as dehiscence, silicone detachment, hematoma, necrosis, and bacterial infection, resulting in poor integration of Integra BMW have also been reported in some instances to increase patient length of stay and total hospitalization cost (17). Synthetic scaffolds, especially PU-based foams, are a promising alternative that is more economical to manufacture at scale; the PU foam Novosorb BTM is $850 for a 100 cm² product. However, polyester-based materials are limited by an autocatalytic, hydrolytic degradation mechanism that proceeds independent of cellular activities (31). Autocatalytic degradation can cause rapid, mid- to late-stage material loss with consequent scaffold pore collapse, inhibition of cell infiltration, and poor wound stenting. More stable polyester chemistries can be used to slow the degradation (86), but this approach increases risk of developing a foreign body fibrous encapsulation response analogous to that caused by non-degradable implants (87).

To maximize the potential of synthetic scaffolds, overcome the shortcomings of PE-URs, and better elucidate structure-function relationships of PTK-URs, we synthesized a series of PTK diols with varied numbers of EG repeats between TK bonds in the polymer backbone. The goal was to tune this class of materials to harness ROS as a biological, cell-produced degradation mechanism (9) while in turn providing antioxidant properties, as oxidative stress is a characteristic of chronic wounds suffering from persistent type 1 immune response (88-90). A series of PTK diols was thus created to enable systematic evaluation of the effect of hydrophilicity on ROS-mediated scaffold degradation/ROS scavenging, immune response, vascularization, and quality of tissue repair. The EG7 formulation, created through a newly developed synthetic strategy, was more hydrophilic than any PTK-UR foams generated previously and emerged as the lead composition.

TK bond density and in vitro ROS reactivity showed an inverse correlation across the PTK-UR scaffold series. Decreased TK content with EG7, relative to the rest of the EG series, actually yielded a higher rate of degradation and mass loss, highlighting the importance of scaffold hydrophilicity on the rate of (oxidative) degradation. This is likely due to increased exposure and susceptibility of TK bonds to cleavage by strongly polar oxidative species in a hydrated environment. The differences in radical-scavenging potential between PTK-UR chemistries was diminished in organic solvents (DPPH assay performed in 80% ethanol) that more readily solvate the hydrophobic PTK-URs. It should be noted that, despite a similar overall EG content in EG2 and EG7 PTK scaffolds (20 vs 21 EG repeats per diol), significant (P<0.001) differences in swelling ratio (208.72% vs 619.72% for EG2 and EG7, respectively) were observed. This is likely due to the inherent hydrophobicity of TK bonds and highlights the importance of having strongly hydrophilic spacer groups along the polymer backbone to ensure accessibility of the TK bond within aqueous environments. These results were also consistent with our previous observation that highly hydrated PEG-maleimide PTK hydrogels subcutaneously implanted in mice underwent oxidative degradation and facilitated effective tissue infiltration (36). The better ROS reactivity of the EG7 scaffolds in aqueous environments could also be connected to the theoretical Log P values for the PTK diols; only the EG7 PTK diol chemistry showed a Log P value below zero (indicating partition preference of water over octanol), whereas the remainder of the EG series had Log P greater than 1. In vivo analysis of EG7 PTK-UR and 900t PE-UR show significantly (P<0.01) lower ROS concentration in thioketal containing scaffold treatments in mice and reduction in O-8HdG staining by IHC in histological wound samples from pigs. The reduced ROS concentration and cell damage in PTK scaffolds highlights the antioxidant properties of PTK-based foams. This likely contributes to the overall healing benefits observed with EG7 foams, as lowering oxidative stress can reduce NF-κB driven expression of inflammatory genes and cytokines that hinder nonhealing wounds (91, 92).

Differences in scaffold-tissue integration between EG0/EG1/EG2 PTK-UR scaffolds and EG7 PTK-URs were pronounced when implanted in porcine excisional skin wounds. Despite similarities is porosity, pore size, and dynamic mechanical compressive properties, all physical features that affect tissue response (45, 93, 94), EG7 PTK-UR scaffolds were more evenly distributed throughout the thickness of the wound (3.1±0.4 mm) while there was lower tissue integration (<2 mm) of more hydrophobic PTK-UR scaffolds. This is likely due to a combination of scaffold swelling, and degradation, processes which facilitate tissue infiltration into the foam pores. EG7 PTK-UR scaffolds had greater vascularization, which may be attributable to foam swelling/degradation and also to the relatively low expression of myeloperoxidase, an antiangiogenic enzyme highly expressed in chronic wounds (95). EG7 PTK-URs were also associated with higher expression of growth factors such as PDGF (96, 97) and TGFB3 (98, 99) (FIGS. 18A-E), which positively impact endothelial cell proliferation, migration, and vessel maturation. In sum, we concluded that the favorable wound healing response of the EG7 vs more hydrophobic PTK-UR scaffolds is related to mechanisms such as an improved biological response to the more swollen scaffold architecture, the composition of the protein adsorbate (63, 100), better ROS reactivity and scavenging, and faster breakdown and clearance of more hydrophilic PTK-UR material degradation products. The EG7 PTK-UR scaffolds were also shown, like previous thioketal materials (34) to provide a relatively constant rate of in vivo breakdown without signs of an autocatalytic degradation mechanism, thereby mechanically supporting cell infiltration and slowly resorbing away within slow-to-heal wounds.

Another central finding of the study was that the EG7 PTK-UR biomaterial triggered a more desirable host immune response and time course of resolution relative to more hydrophobic PTK-URs. Tissue damage and biomaterial implantation are inextricably linked with initial extravasation of circulating neutrophils, monocytes, and macrophages to the site of injury (101). These inflammatory first responders generate ROS such as ·OH, H₂O₂, ClO—, and O₂— as part of the innate immune response (102). The density of macrophages, T cells, and myofibroblasts, cell types responsible for non-degradable implant fibrous encapsulation (59, 61, 103), were all significantly (P<0.05) decreased in the EG7 scaffold formulations. Active recruitment of neutrophils, monocytes, macrophages, and dendritic cells to the site of biomaterial implantation can propagate inflammation and foreign body response resulting in tissue and implant structural damage (104). We observed that EG7 PTK scaffolds were associated with significantly (P<0.05) fewer CCR7 positive M1 macrophages and FBGCs relative to more hydrophobic PTK-URs. This agrees with previous observations that hydrophilic and anionic surfaces can cause biomaterial-adherent macrophage apoptosis and lower macrophage fusion, potentially limiting the deleterious effects of pro-inflammatory macrophages and reducing formation of FBGCs (105). Dendritic cells (DCs) serve as an important bridge between innate and adaptive immunity by interacting with biomaterials through pathogen recognition receptors (PPR), leading to downstream antigen presentation and activation of T cells. Hydrophilic surfaces can also limit DC adhesion and maturation and consequently reduce pro-inflammatory T cell response (106, 107). Indeed, our subcutaneous implant mouse studies showed that EG7 PTK scaffolds had decreased number of infiltrating DCs compared to the more hydrophobic EG2 PTK-UR. These collective data suggest that the more hydrophobic scaffolds elicit a larger magnitude and more sustained inflammatory response compared to EG7 PTK-UR foams and that both initial recruitment and temporal evolution of the immune response that have characteristic implications in the wound healing potential of synthetic biomaterials.

Wound closure by re-epithelialization is a key aspect of wound healing that is typically a primary outcome in clinical studies, as re-establishment of skin epidermal barrier is associated with reduced risk for infection and patient morbidity. A moist environment within chronic wounds has been linked with positive healing outcomes, as opposed to slower rates of wound closure and incomplete resurfacing seen in desiccated wounds (108). The ability of the EG7 PTK-UR to maintain a more hydrated wound environment may have also contributed to the significantly higher (P<0.01) epithelial resurfacing of EG7 PTK treated wounds compared to more hydrophobic foams. In addition to the physical properties of EG7 PTK-UR foams, these scaffolds also recruited significantly higher (2.7-fold, (P<0.001)) numbers of γδ T cells compared to EG2 PTK scaffolds when implanted in of the mouse subcutaneous space. This T cell subpopulation stimulates secretion of growth factors (66) that promote keratinocyte migration and re-epithelialization (53), several of which were upregulated in EG7 PTK-UR treated wounds [IGF1 (2-fold), TGFB3 (2-fold), and PDGF (3-fold)].

To establish the therapeutic potential of the EG7 PTK-UR scaffolds more thoroughly in wound healing, we benchmarked against commercially available Integra BWM and NovoSorb BTM in pig skin wounds. Though similar rates of wound closure were observed between Integra and EG7 PTK-UR treatments, significantly (P<0.0001) higher blood perfusion was seen in wounds treated with EG7 PTK-UR, indicating that neovascularization is a competitive advantage of EG7 PTK-UR scaffolds that may come more into play and yield superior performance relative to Integra for treatment of ischemic wounds. A slower degradation profile of NovoSorb was qualitatively inferred by the presence of a relatively large proportion of scaffold material in the wound after 31 days, which could potentially cause a prolonged host foreign body response. The persistence of NovoSorb in our studies was also consistent with previous observation of NovoSorb scaffold remnants in human skin biopsies taken 12 months post implantation (109). The relatively high in vivo stability of NovoSorb may at least partially explain the relatively higher expression of pro-inflammatory cytokines genes such as ILIB (3.1-fold), TNFA (2.1-fold), NOS2 (4.5-fold), CXCL8 (12-fold), and CSF2 (5.9-fold) observed in NovoSorb treated wounds compared to EG7 PTK-UR treatments. In comparison to architecturally and synthetically analogous NovoSorb foams, EG7 PTK-UR foams explanted from wounds at day 31 had significantly upregulated expression of anti-inflammatory interleukins IL13 (2.8-fold) and IL33 (3.2-fold), which are known to induce a tissue repair phenotype in macrophages (110, 111).

Surprisingly, we observed significantly lower arginase-1 (Arg-1) expression (10-fold), a marker often used to characterize the anti-inflammatory M2 macrophage phenotype, in EG7 PTK-UR wounds relative to NovoSorb. Arg-1 IHC of wound tissue revealed lower but not the absence of Arg-1 expressing macrophages in EG7 PTK-UR treated wounds. Intense Arg-1 staining was, however, observed colocalized with FBGCs surrounding NovoSorb scaffold remnants. Despite the importance of Arg-1 metabolism in macrophage polarization, elevated expression of Arg-1 has been reported in ischemic porcine wounds (112) and in human venous leg ulcers (112). In this context, high arginase activity has been linked to fibrosis (113), suggesting disparate biological ramifications of Arg-1 expressing macrophages. Related to the fibrosis phenotype, we observed higher TGFB3/TGFB1 expression ratio in EG7 PTK-UR treated wounds compared to Novosorb. It is known that TGFB3 can promote scarless healing in mice while upregulated TGFB1 is associated with excess ECM deposition and fibrosis (114).

To better recapitulate a clinical scenario with poorly healing skin wounds that would require treatment with a biomaterial, we utilized a previously optimized porcine model in which excisional wounds are created in raised flaps that ischemic due to being surgically separated from the underlying fascia and lateral regions of skin (115). The raise skin flaps have reduced blood flow relative to surrounding normal skin as measured by Doppler imaging (35). This model captures aspects of ischemic-reperfusion injury, which can cause formation of chronic wounds in scenarios where there are underlying vascular insufficiencies (8). Ischemia-reperfusion triggers formation of free oxygen radicals that cause oxidative stress and cellular damage in the tissue. Thus, we chose this model as a promising platform for demonstrating the benefits of the anti-oxidant PTK-UR materials. In this model, both EG7 PTK-UR and NovoSorb scaffolds promoted cell infiltration, but EG7 PTK-UR scaffolds significantly improved rate of wound closure and higher percent re-epithelialization at the endpoint compared to NovoSorb. Treatment effect differences on re-epithelialization between EG7 PTK-UR and Novosorb BTM were better resolved compared to the non-ischemic wound model. Similar to the wound healing profile seen in non-ischemic wounds, EG7 PTK-UR treatment improved resolution of inflammation based on IHC for MPO positive neutrophils and CCR7 positive macrophages. These data collectively suggest that the improved quality of tissue repair observed in full thickness skin wounds on healthy pigs can be extended to ischemic skin wounds, a better recapitulation of chronic non-healing phenotypes observed in patients.

There were some notable limitations of the promising studies presented herein that will be considered in future product development work. First, testing the regenerative capacity of PTK-UR in acute wounds created on healthy pigs gives important but still limited insight into performance in chronic human skin wounds. Chronic wounds can be associated with more permanent ischemia, infection, and potentially higher oxidative stress that may over-accelerate the degradation of EG7 PTK-UR scaffolds. Additional tuning of mechanical properties to compensate for accelerated degradation maybe be required to effectively treat chronic wounds. The presence of a non-degradable, temporary membrane to create a bilaminar architecture such as used in Integra and Novosorb should also be a future consideration. Integration of an upper, removable layer on EG7 PTK-UR my help to better protect against moisture loss and bacterial infections as well as improve mechanical properties to reduce contraction of the underlying dermal layer. However, based on our observation with Novosorb, the ideal timing of the upper layer removal is also expected to be wound size- and healing rate-dependent and must be carefully monitored to ensure that the upper layer does not actually delay re-epithelialization and slow closure. Another consideration is the small wound size used in the study. Reepithelization of larger wounds with EG7 PTK treatment alone may be challenging and would mostly likely be paired with the application of split-thickness skin graft or autologous keratinocyte transplant (116). The ischemic wound model utilized to study differences in polyurethane scaffold assisted wound healing of PTK-UR and NovoSorb matrices provides a robust model to study healing kinetics of wounds with compromised vascularity. While this model provides robust ischemia, it does not fully capture the background conditions that exist in many chronic wound patients (systemic vascular disease, diabetes, etc.), and it may be advantageous to carry out future studies using larger sized excisional wounds, to induce diabetes, or to feed a high fat diet in the model animals prior to skin wounding.

In summary, we have developed a novel EG7 PTK-UR foam dressing that facilitates bulk tissue-scaffold integration, robust cellular and vascular infiltration, and wound resurfacing. These implants induce a moderate inflammatory response that effectively transitions to a pro-healing phenotype, in addition to promoting ECM deposition, re-epithelialization, and remodeling. When tested against clinically approved materials in porcine excisional wounds, EG7 PTK-UR had higher vascularization and similar wound closure to the gold standard biologic material Integra BWM and significantly outperformed the synthetic polyester-based foam NovoSorb.

### Materials and Methods

### Study Design

The current studies tested a library of ROS-degradable PTK-UR scaffolds in a porcine wound model. All surgical procedures were reviewed and approved by Vanderbilt University Institution and Animal Care and Use Committee. We created 28, 2×1 cm full thickness wounds on the dorsal region of (healthy) adolescent Yorkshire female pigs (32 kg). Wounds were spaced ≥2 cm apart to avoid collateral effects while enabling screening of multiple biomaterial formulations, (3-4 wounds per treatment groups), as previously reported (35, 117, 118). Treatment placement was randomized to avoid anatomical bias. To evaluate host response and early to mid-stage immune response to the implanted scaffolds, pigs were euthanized, and wounds harvested at 10 days post scaffold implantation. Full-thickness biopsies from within the wound margins were collected from anesthetized pigs prior to euthanasia for gene expression analysis. Wounds were harvested at 24 days post-implant to further evaluate wound re-epithelialization and other histological features of within the polyurethane (PU) (PE-UR controls and PTK-UR library) scaffolds (n=6-10). In a third experiment, we treated porcine full thickness wounds (n=6-9) with our lead PTK-UR formulation, Integra BWM, and NovoSorb BTM and ended the study after 31 days. Wound closure and blood perfusion were non-invasively measured over the time course, while tissue samples for gene expression and histological evaluation were collected upon animal euthanasia. In a 4^{th} experiment, 4 ischemic flaps were raised in a pig, and two 1 cm×1 cm wounds were created per flap. Wounds were randomized for treatment with either lead PTK-UR formulation or NovoSorb. Blood perfusion was measured over time prior to study endpoint (day 17), when wound tissues were harvested for histological analysis.

For all pig studies, the wound dressings were identical across all treatments and changed three times a week. Pigs received weekly antibiotic (Excede; Zoetis) injections to address potential bacterial infections along with analgesics (Buprenex, Fentanyl) to manage pain. Pre-mature loss of scaffolds from the wounds due to mechanical disturbance by the animal prior to the pre-determined end point was a criterion for exclusion of that sample from outcome analysis. Scoring of wound histology was done by a histopathologist blinded to the treatment. To further evaluate scaffold infiltrating immune cell populations, we implanted two PU scaffolds (same PTK chemistry) in the ventral region of mice (n=6 mice per scaffold type). Animals were then euthanized, and scaffolds were retrieved from the subcutaneous space at days 7, 14 and 21 post scaffold implantation. Cells were isolated from the explanted scaffolds (cells pooled from 2 scaffolds from the same animal) and profiled based on cell surface and intracellular markers using flow cytometry. Mice were similarly implanted with 900t PE-UR and EG7 PTK-UR scaffolds to assess relative ROS levels associated with each scaffold chemistry in vivo, 4 days after implantation. All mice received analgesics (Carprofen) every 24 hours for the first 72 hours post-surgery to manage pain. None of the treatments resulted in animal weight loss or mortality during the course of the experiment.

### Synthesis of EG0 (BDT), EG1 (MEE), and EG2 (EDDT) PTK Diols

All chemical reagents were purchased form Sigma Aldrich. EG0, EG1 and EG2 PTK diols were synthesized as previously published by Martin et al. (34, 36). 1,4 butanedithiol (BDT, EG0), mercaptoethyl ether (MEE, EG1), and 2,2′-(ethylenedioxy) diethanethiol (EDDT, EG2) were polymerized with dimethoxy propane (DMP) through a condensation reaction using p-toluene sulphonic acid (pTSA) as a catalyst (FIG. 21). First, pTSA was dissolved in concentrated hydrochloric acid (HCl) and heated to 50° C. The acid was allowed to cool, and pTSA crystals were collected, rinsed with cold HCl, and vacuum dried for 2 hours. A tri-neck flask attached with an addition funnel was charged with anhydrous acetonitrile (ACN) and dithiol monomers (1 mol eq, 0.400 mol) stirred continuously and heated to 80° C. followed by the addition of re-crystallized pTSA (0.005 mol eq, 0.002 mol). DMP (0.83 mol eq, 0.334 mol) dissolved in acetonitrile was added dropwise to the monomer mixture and allowed to stir overnight. The crude polymer solution was concentrated under rotary evaporation, precipitated in cold ethanol, and vacuum dried. To further carry out hydroxyl functionalization, PTK dithiol polymers (1 mol eq, 0.060 mol) were dissolved in anhydrous tetrahydrofuran (THF) followed by the addition of CsCO₃ (5 mol eq, 0.15 mol) and 2-bromoethanol (5 mol eq, 0.15 mol) and allowed to react for 4 hours. The resulting PTK diol polymers was filtered, concentrated, precipitated in ethanol, and vacuum dried. Respective PTK diol polymers were dissolved in deuterated chloroform (CDCl₃) and their compositions were evaluated using 400 Hz ¹H nuclear magnetic resonance (NMR) spectroscopy (400 MHz Bruker); chemical shifts were reported as δ values in ppm relative to deuterated CDCl₃ (δ=7.26).
EG0 (BDT) PTK diol; δ=1.60 (s, 6H, —S—C(CH₃)₂—S—), δ=1.65-1.69 (m, 4H, —S—CH₂—(CH₂)₂—CH₂—S—), δ=2.58-2.61 (t, 4H, —S—CH₂—(CH₂)₂—CH₂—S—), δ=2.69-2.73 (t, 2H, —S—CH₂—CH₂—OH), δ=3.68-3.72 (t, 2H, —S—CH₂—CH₂—OH).
EG1 (MEE) PTK diol; δ=1.60 (s, 6H, —S—C(CH₃)₂—S—), δ=2.72-2.76 (t, 2H, —S—CH₂—CH₂—OH), δ=2.78-2.82 (t, 4H, —S—CH₂—CH₂—O—CH₂—CH₂—S—), δ=3.58-3.64 (m, 4H, —S—CH₂—CH₂—O—CH₂—CH₂—S—), δ=3.68-3.74 (t, 2H, —S—CH₂—CH₂—OH).
EG2 (EDDT) PTK diol; δ=1.60 (s, 6H, —S—C(CH₃)₂—S—), δ=2.72-2.74 (t, 2H, —S—CH₂—CH₂—OH), δ=2.77-2.80 (t, 4H, —S—CH₂—[CH₂—O—CH₂]2—CH₂—S—), δ=3.56-3.60 (m, 8H, —S—CH₂—[CH₂—O—CH₂]₂—CH₂—S—), δ=3.68-3.74 (t, 2H, —S—CH₂—CH₂—OH)

### Synthesis of EG7 PTK Diols

Synthesis of thioketal diethylamine was adapted from previously published work as shown in FIG. 22 (41, 119).

Mercaptoethyl trifluoroacetamide [1]. Cysteamine (1 mol eq, 0.47 mol) was dissolved in anhydrous methanol under nitrogen flow, followed by the addition of triethylamine (1.5 mol eq, 0.70 mol). Ethyl trifluoroacetate (1.2 mol eq, 0.56 mol) was added to the solution dropwise and allowed to stir on ice for 1 hour. The reaction mixture was brought to room temperature and stirred overnight. The concentrated reaction mixture was dissolved in water, extracted into ethyl acetate, dried over anhydrous magnesium sulfate, and concentrated in vacuo. The crude product was further purified by silica gel chromatography using a gradient of 100 to 70:30 ethyl acetate:hexane. The product (Compound [1], (FIG. 22), a colorless liquid, was analyzed using 400 Hz ¹H NMR; chemical shifts were reported as 6 values in ppm relative to deuterated CDCl₃ (δ=7.26) as follows; δ=2.7-2.76 (q, 2H, —NH—CH₂—CH₂—SH), 3.52-3.56 (t, 2H, —NH—CH₂—CH₂—SH), 7.01 (s, 1H, —NH—)

Thioketal diethyltrifloroacetamide [2]. Compound [1] (2.2 mol eq, 0.42 mol) was dissolved in acetonitrile and charged with N₂ gas followed by the addition of catalytic amounts of bismuth chloride (BiCl₃) (0.025 mol eq., 0.005 mol). DMP (1 mol eq., 0.192 mol) dissolved in acetonitrile was added to the flask dropwise and reaction was allowed to proceed at room temperature for 24 hours. Acetonitrile was removed from the mixture via rotary evaporation, and the crude product was filtered through silica in a mixture of 70:30 hexane:ethyl acetate for additional purification. The product (compound [2], (FIG. 22), a white solid, was analyzed through 400 Hz ¹H NMR in CDCl₃. Chemical shifts were reported as 6 values in ppm relative to deuterated CDCl₃ (δ=7.26) as follows; δ=1.60 (s, 6H, —S—C(CH₃)₂—S—), 2.8-2.85 (t, 4H, —NH—CH₂—CH₂—S—), 3.56-3.61 (m, 4H, —NH—CH₂—CH₂—S—), 6.8 (s, 2H, —NH—)

Thioketal diethylamine [3]. Compound [2] (1 mol eq, 129 mol) was dissolved in minimal volume of methanol, and saponified in an aqueous 6M NaOH solution at room temperature for 4 hours. The resulting product was extracted into dichloromethane, dried over anhydrous magnesium sulfate, and concentrated under vacuum to obtain, a pale-yellow colored oil. Thioketal diamine crosslinker (Compound [3]) was then analyzed through 400 Hz ¹H NMR in CDCl₃ (δ=7.26) as follows; δ=1.53 (s, 6H), —S—C(CH₃)₂—S—), 2.58-2.62 (t, 4H H₂N—CH₂—CH₂—S—), 2.66-2,69 (m, 4H, H₂N—CH₂—CH₂—S—).

PEG-NPC: Activated PEG 400 was synthesized as previously described (120). Briefly, PEG 400 (Mₙ=400 Da) was dried at 80° C. under vacuum for 24 hours. Dried PEG 400 (1 mol eq, 0.10 mol) was dissolved in anhydrous dichloromethane with triethylamine (1.2 mol eq., 0.12 mol) on ice. In a separate round bottom flask, p-nitrophenyl chloroformate (1.1 mol eq., 0.11 mol) was dissolved in anhydrous dichloromethane and added dropwise to the PEG400/triethylamine mixture. The reaction was mixed 30 minutes on ice and allowed to react for an additional 24 hours at room temperature. Triethylamine hydrochloride salts that were formed during the reaction were removed via filtration and the product was concentrated on a rotary evaporator. This step yielded a mixture of monofunctionalized PEG nitrophenylcarbonate (OH-PEG-NPC) and homobifunctional PEG 400 dinitrophenylcarbonate (NPC-PEG-NPC).

The product was analyzed through 400 Hz ¹H NMR in DMSO (δ=2.5) as follows; δ=3.5-3.6 (m, 28H, —[O—CH₂—CH₂]—), 3.71 (m, 2H, —O—C(═O)—O—CH₂—CH₂—O—), 4.4 (m, 2H, —O—C(═O)—O—CH₂—CH₂—O—), 7.6 (d, 2H, phenyl protons meta to nitro), 8.3 (d, 2H, phenyl protons ortho to nitro).

EG7 PTK diol. Activated PEG nitrophenylcarbonate mixture (OH-PEG-NPC/NPC-PEG-NPC, 2.4 mol eq, 0.034 mol) dissolved in anhydrous dichloromethane was added dropwise to thioketal diamine crosslinker (Compound [3], 1 mol eq, 0.014 mol) and triethylamine (2.4 mol eq, 0.034 mol) which was allowed to react for 4 hours. The crude mixture was then precipitated in cold diethyl ether to remove p-nitrophenol impurities, and further purified through silica column chromatography. Crude product was loaded onto a silica packed column, and fractions of clean product were eluted using a mixture of dichloromethane and methanol at gradients of 0 to 10% methanol.

The fractions were then dried in vacuo and the product (50% yield) was analyzed through 400 Hz ¹H NMR in DMSO (δ=2.5) as follows; δ=1.54 (s, 12H, —S—C(CH₃)₂—S—), 2.58-2.62 (t, 8H, CH₂—S—C(CH₃)₂—S—CH₂—), 3.11-3.16 (m, 8H, —S—CH₂—CH₂—NH—), 3.49-3.53 (m, 94H, —[O—CH₂—CH₂]—), 3.61 (t, 8H, O—C(═O)—O—CH₂—CH₂—O), 4.17 (t, 4H, O—C(═O)—O—CH₂—CH₂—O), 7.34 (s, 3H, —NH—).

### Polyester Polyol Synthesis

Trifunctional polyester polyols (900t and 1500t) were synthesized as previously published (121). Briefly, glycerol was dried for 48 hours at 80° C. under vacuum. Dried glycerol was added to a ti-neck flask, charged with nitrogen followed by the addition of 60% E-caprolactone (CL), 30% glycolide (GA), and 10% D, L-lactide (LA) along with a stannous octoate catalyst to yield a 900 Da triol and 1500 Da triol.

### Determination of Molecular Weight and Hydroxyl Number

Polymer molecular weights (M.) and dispersities (D) were determined through dimethylformamide gel permeation chromatography (Agilent 1260 Infinity) with inline refractive index and light scattering detectors (Wyatt miniDAWN Treos), using Astra software and dn/dc values obtained from a refractometer (Anton Paar Abbemat 300). The hydroxyl (OH) number of the resultant polymeric diols was quantitatively determined via ¹⁹F NMR spectroscopy using a method was adapted from Moghimi et al. (122). Briefly, 0.03 mmol of PTK diol was dissolved in deuterated acetone and reacted with 0.3 mmol of reagent 4-fluorophenyl isocyanate (4-F-Ph-NCO) for 15 mins at room temperature in the presence of 0.1 wt. % dibutyltin dilaurate (DBTDL) used as a catalyst and 0.081 mmol of u, u, u-trifluorotoluene (CF₃-Ph) as internal standard. The ¹⁹F NMR spectrum in deuterated acetone was recorded as follows; δ=−63.23 (s, 3F, CF₃-Ph), δ=−117.6 (m, 1F, 4-F-Ph-NCO), δ=−122.2 (s, 2F, 4-F-Ph-NH—CO—O-PTK). Theoretical hydroxyl number of each PTK diol was calculated by:

$$Mn = \frac{56 , 100 * f}{OH \#}$$

where 56,100 represents the molecular weight of KOH in mg/mol, ƒ represents the hydroxyl functionality of the PTK (two for a linear homobifunctional PTK polymer) and Mₙ represents the number-average of the PTK polymers.

The logarithmic partition coefficient (Log P) of respective PTK diols were obtained computationally using the log P plugin for Marvin (ChemAxon). The octanol-water partition coefficient is commonly used to measure relative solubilities of a polymer.

### Fourier Transform Infrared Spectroscopy (FTIR) and Differential Scanning Calorimetry (DSC)

10 uL of each PTK diol was applied in a thin layer between two 25×4 mm potassium bromide (KBr) glass windows and loaded into a Bruker Tensor 27 FTIR Spectrometer. Absorbance at 3400 cm⁻¹ was used to identify OH groups for each PTK diol. In addition, absorbance peaks at 3330 cm¹ (N—H stretching) and 1630 cm¹ (carbonyl groups in urea bonds C═O) were identified to confirm the presence of NHCOO (urethane) bonds in EG7 PTK diols.

For glass transition temperature (T_{g}) analysis using differential scanning calorimetry (Q200, TA Instruments), samples ranging from 5 to 9 mg were heated from −80.0° C. to 20.0° C. at a rate of 10° C. min⁻¹. Samples were then cooled to −80.0° C. at a rate of 10° C. min⁻¹, and heated a second time to 30° C. at a rate of 10° C. min⁻¹. All transitions were obtained from the second heating run.

### Urethane Foam Fabrication

PTK and PE urethane (UR) scaffolds were fabricated using reactive liquid molding. Briefly, PTK-diols were mixed with calcium stearate (pore opener), TEGOAMIN33 (catalyst, Evonik), water (blowing agent), and sulfated castor oil (pore stabilizer) for 30 seconds at 3300 rpm in a Speed Mixer (Hauschild DAC 150 FVZ-L Speed Mixer (FlackTek, Inc., Landrum, SC). Lysine triisocyanate (LTI) was added and mixed for an additional 30 s at 3300 rpm. The mixture was allowed to rise, set, and harden overnight before the scaffolds were demolded and trimmed to shape. The target isocyanate: hydroxyl ratio (NCO OH) was set to 115:100 where the OH equivalents were obtained from polyol's molecular weight and OH number obtained from ¹⁹F NMR analysis. The mass of each component for each scaffold formulation denoted as parts per hundred-part polyol (PPHP) was individually optimized and are outlined in Table 1.

### Urethane Film Synthesis and Contact Angle Measurement

PE-UR and PTK-UR films (1 mm thick) were synthesized by combining polymeric diols and bismuth neodecanoate (a gelling catalyst) in a 22 mm polyethylene mold for 30 seconds at 3300 rpm. LTI was then added to this mixture and mixed for additional 60 seconds. Urethane films were allowed to cure for 24 hours at room temperature before being demolded and punched into 4 mm circles using a biopsy punch.

Contact angle measurements were carried out using a goniometer (Rame-Hart, Model A-100). A 4 μl water droplet was dispensed onto the surface of the urethane film and allowed to equilibrate for 10 mins. Bilateral contact angle measurements were obtained for each film, and averaged values across three different batches of urethane films were recorded for each formulation.

### Sol Fraction

Fabricated scaffolds were weighed to obtain the initial dry mass (Mₒ) and incubated in dichloromethane for 24 hours. Scaffolds were then removed from solvent, air dried and weighed to obtain the final mass (M_{ƒ}). Soluble fraction (ƒₛ) was calculated using the following equation:

$${f}_{s} = \frac{{M}_{o} - {M}_{f}}{{M}_{o}} * 100$$

### Swell Ratio

To determine the swelling ratio of PTK-UR and PE-UR scaffolds, 10 mg (dry mass, M_{d}) scaffolds were incubated in phosphate-buffered saline (PBS) overnight and weighed to obtain swollen mass (Mₛ). The swell ratio was calculated using the following equation:

$$\% Swelling = \frac{{M}_{s} - {M}_{d}}{{M}_{d}} * 100$$

### Dynamic Mechanical Analysis

Mechanical properties of PTK-UR and PE-UR scaffolds were measured in compression using the TA Q800 Dynamic Mechanical Analyzer. 6×6 mm cylindrical scaffold punches were hydrated in PBS for 24 hours prior to measurement. Scaffolds were longitudinally compressed under a preload force of 0.1N at 10% strain min⁻¹ until 60% strain was reached. The longitudinal compression was repeated three times and values averaged per sample. The scaffolds were not rehydrated between these compression cycles. The Young's modulus for each sample was calculated from the linear region of the stress-strain curve after initial toe-in. A total of 3 scaffold pieces across three separate batches were averaged to obtain the Young's modulus of PU foams.

### In Vitro Degradation of PTK-UR Scaffolds

Pre-washed and dried 10 mg scaffold pieces (Mₒ) fabricated from PE or PTK diols were incubated in PBS and increasing concentrations of hydrogen peroxide/cobalt chloride (H₂O₂/CoCl₂) solutions to generate hydroxyl radical; 0.2% H₂O₂/0.001 M CoCl₂, 2% H₂O₂/0.01 M CoCl₂, or 20% H₂O₂/0.1 M CoCl₂. Scaffolds were incubated in 2 mL of treatment media for up to 30 days with media replaced every other day. At pre-determined time points, scaffolds were removed from the treatment media, rinsed in water, dried, and weighed to obtain the final remaining mass (Mₜ) of the scaffold. Three independent scaffold pieces were tested at each time point. Degradation was recorded as percent mass loss for each time point using the following equation:

$$\% Mass Remaining = \frac{{M}_{t}}{{M}_{o}} * 100$$

### Mathematical Modeling of PTK-UR Scaffold Degradation (FIG. 27)

MATLAB (MathWorks) was used to fit the oxidative degradation kinetics of PTK-UR scaffolds to a first order mathematical model based on H₂O₂ concentration using the following equation:

$$\frac{{M}_{t}}{{M}_{o}} = {e}^{- kt}$$

where Mₜ represents the final mass of scaffold time at time t, Mₒ represents the initial scaffold mass, and k represents the degradation rate constant. Non-linear regression was used to fit the experimentally obtained scaffold degradation data to the first order mathematical model to determine the degradation rate constant for each scaffold with respect to the degradation media concentration.

### Scanning Electron Microscopy of Urethane Scaffolds

Scaffold pieces (60-80 mg) fabricated using PE and PTK diols were incubated in PBS or 2% H₂O₂ with 0.01M CoCl₂ for up to 15 days. At pre-determined intervals, scaffolds were removed from their respective treatments and washed in a series of graded ethanol solutions (30%, 50%, 70%, 80%, 90%, 95% and 100%) for 10 minutes to remove the aqueous content of the degradation media. Scaffolds were then dried via critical point drying and weighed to obtain final scaffold mass. Dry scaffold pieces were then sputter coated (Cressington Scientific Instruments) with gold and imaged on a scanning electron microscope (Merlin, Carl Zeiss) using a Everhart-Thomley secondary electron detector at a beam voltage of 3.00 keV or 10 keV. Pore sizes from SEM images were quantified using ImageJ software.

### DPPH Radical Scavenging Assay

The radical scavenging potential of PE-UR and PTK-UR scaffolds were assessed using a 1,1-diphenyl-2-picrylhydrazyl (DPPH, Sigma Aldrich) assay (123). DPPH was dissolved in a mixture of 80:20 ethanol and H₂O (v/v %) at a concentration of 200 μM. Pre-fabricated PE and PTK urethane scaffolds (10 mg) were treated with 2 mL of DPPH solution, and incubated at 37° C. on an orbital shaker for 36 hours. 100 μL of incubation solution was sampled at 1, 2, 3, 6, 12, 24, and 36 hour time points and the absorbance (Aₛₐₘₚₗₑ) was measured at 517 nm and compared to control (no scaffold) DPPH solution absorbance (A_{control}). The scavenging potential is reported as following:

$$\% Inhibition = \frac{\begin{matrix}{A}_{Control} & {A}_{Sample}\end{matrix}}{{A}_{Control}} * 100$$

### ROS Cell Protection Assay

Cell protection against cytotoxic levels of hydrogen peroxide was tested in NIH 3T3 fibroblasts. Briefly 10,000 cells were seeded in a 96 well plate in complete media (DMEM supplemented with 10% fetal bovine serum) and allowed to adhere overnight. Growth media was removed, and cells were sequentially treated with 100 μL of oxidative media (25 μM H₂O₂ or 50 μM H₂O₂) and 100 μL of test polyol solution and allowed to grow for an additional 24 hours. Post treatment, cell viability was assessed using CellTitre-Glo Luminescent Cell Viability Assay (Promega) and quantified by measuring bioluminescence on an IVIS 200 (PerkinElmer). Signal from treatment groups was normalized to untreated control cells and expressed as relative cell viability. The overall thioketal molar content (0.5 μmol) for each PTK diol test group was kept constant and control PE triol controls were formulated at 0.5 mg/mL to match the highest PTK diol mass.

### Porcine Excisional Wound Model and PTK-UR Scaffold Screening

All surgical procedures were reviewed and approved by Vanderbilt University Institutional Animal Care and Use Committee. Adolescent female Yorkshire pigs (n=3) were anesthetized with a cocktail of telazol (4.4 mg/kg), ketamine (2.2 mg/kg), and xylazine (2.2 mg/kg) administered intramuscularly and maintained under isoflurane for the duration of the surgery. Dorsal skin was shaved and disinfected using 70% ethanol, chlorohexidine, and betadine washes. Twenty-eight, full thickness 2×1 cm excisional wounds were created on the dorsum of the pig with two parallel rows of wounds produced on each side of the spine. Ethylene oxide-sterilized scaffold pieces were trimmed to 0.2×2×1 cm and implanted into the wounds after hemostasis was achieved. Each pig received all 5 treatments, placement was randomized to avoid anatomical bias. Following implantation of the test materials, wounds were covered with layerings of Mepilex Transfer (Molnlycke Healthcare), Mextra absorbent pad (Molnlycke), OpSite adhesive (Smith&Nephew), MediChoice Tubular Net Bandage (Owens & Minor), and Vetwrap (3M) bandaging. Following surgery, dressings were changed every 2-3 days under anesthesia. Antibiotics (Excede; Zoetis) were administered weekly during the study period. Buprenex was given immediately post-surgery, and analgesia was maintained for 7 days post-op using a transdermal Fentanyl patch (50 mcg/h) applied at the time of surgery and changed every 2-3 days. In independent studies, animals were euthanized at either 10 or 24 days post-surgery; wound samples were collected in anesthetized pigs using 2 mm biopsy punches for gene expression analysis. Post euthanasia, the remaining wound site was excised with in the wide margins for histological analyses.

### Porcine Testing of PTK-UR Against Clinical Controls

Integra Bilayer Wound Matrix (BWM) (Integra Life Sciences) was rinsed in sterile saline, trimmed to 2×1 cm pieces, and placed matrix side down into the excisional bed. The protective silicone layer was removed 14 days post application, according to manufacturer's guidance. NovoSorb Biodegradable Temporizing Matrix (BTM) (PolyNovo) scaffolds were trimmed to 2×1 cm pieces and applied matrix side down into the full thickness skin wound. Sealing polyurethane membrane was removed 21 days post application according to manufacturer's guidance. EG7 PTK-UR scaffolds were similarly applied. Treatments were randomized with regards to wound location to avoid any anatomical bias. The wounds were dressed and secured with additional bandaging as described above. Pigs were euthanized 31 days post-surgery and wound samples were collected for histology and gene expression analysis as previously described.

### Testing PTK-UR and NovoSorb Dermal Matrices in a Porcine Ischemic Wound Model

Under sterile conditions, four pairs of full thickness incisions 15 cm in length and 10 cm wide were created parallel to the spine (two cephalic and two caudal), as previously described (125). Using a cautery, the skin was lifted from the underlying fascia to create bipedicle flaps. The resulting flaps of skin were re-approximated with staples creating four distinct ischemic regions on the dorsal region of the pig (n=1). Two laterally symmetrical 1 cm² full-thickness excisional wounds were then created within each of the four ischemic flaps. 1×1 cm² sized EG7 PTK-UR and NovoSorb BTM scaffolds were implanted into the skin wounds. Each flap received one of each scaffold type, and treatments locations were otherwise randomized to prevent anatomical bias. The wounds were dressed as described above for non-ischemic wounds. The dressings were changed every 3 days. The protective membrane overlayer present on NovoSorb BTM was removed 14 days post application. The pig was euthanized 17 days post-surgery, and wound samples were collected for histological analysis.

### Laser Doppler Perfusion Imaging

A laser doppler imager (moorLDI2-HIR; Moor Instruments) was used to track blood perfusion within the wounds over the duration of the study. Flux images of wounds were obtained after scaffold implantation and subsequently during dressing changes at days 1, 4, 10, 17, 24, and 31 post-surgery. Blood perfusion within a 2×1 cm region of interest as well as adjacent uninjured skin was quantified using Moor analysis software. Data are presented as total flux within the wounds, normalized to the total flux recorded in normal skin.

Blood perfusion within ischemic wounds was also measured using doppler imaging, with data acquired at days 1, 3, 5, 7, 9, 11, 14 and 17 post-surgery. Blood perfusion was quantified within the wound area and presented as total flux.

### Histology and Quantification

Tissue samples were fixed in 10% neutral buffered formalin for 48 hours and were then dehydrated in a graded ethanol series, exposed to xylene, and embedded in paraffin. Tissue sections (5 μm thick) were deparaffinized in gradients of xylene and ethanol and rehydrated in Tris-Buffered Saline/0.1% Tween 20 (TBST) buffer. Masson's trichrome staining and hematoxylin and eosin (H&E) staining were performed according to the manufacturer's recommendation. Antigen retrieval was performed using citrate-based pH 6 antigen retrieval solution (Dako) for 1 minute at 120° C. and allowed to cool to 90° C. Sections were then incubated for 40 minutes in 3% H₂O₂ TBST solution and blocked with protein block (Dako) for 20 minutes. Sections were further incubated in either rabbit anti-human von Willebrand factor (GA52761-2, Dako), mouse anti-human cytokeratin14 (MCA890, Biorad), mouse anti-αSMA (A2547, Sigma Aldrich), rabbit anti-CCR7 (ab32527, abcam), rabbit anti-arginase-1 (ab91279, abcam), rabbit anti-CD3 (ab16669, abcam), rabbit anti-human myeloperoxidase (A039829-2, Dako), or rabbit anti-CD206 (18704.1. AP, Proteintech), mouse anti-9-hydroxy-2′-deoxyguanosine (ab48508) for 60 minutes at room temperature. Secondary antibodies donkey anti-mouse HRP (Envision+/HRP, Dako) or donkey anti-rabbit HRP (Envision+/HRP, Dako) were applied for 30 minutes at room temperature followed by 3,3′-diaminobenzidine (DAB) substrate (Dako) for 5 minutes. Slides were then rinsed in TBST buffer, dehydrated in graded ethanol and xylene solutions, and mounted with Acrytol (manufacturer) mounting media.

Histological sections were imaged and evaluated using Metamorph Imaging Software (Molecular Devices) and ImageJ to assess wound thickness occupied by scaffold and tissue infiltration within the scaffold pores. Wound thickness occupied by scaffold was defined as the longitudinal thickness of the granulation tissue occupied by the bulk scaffold/scaffold remnants. Tissue infiltration was defined as percent area occupied by new granulation tissue within the scaffold pores, averaged across 3 fields of view per sample. Using Metamorph imaging software, Brown pixels (positive immunoreactivity for DAB) were quantified for each IHC marker (CytoK14, CD206, MPO, CD3 and vWF). Epithelialization was quantified as percent wound length covered by K14-positive epidermal layer. vWF, MPO, CD206, and CD3 were quantified and expressed as percent region of interest (ROI) area occupied by positive brown pixels. A minimum of three fields of view per wound sample were analyzed and quantified. Using ImageJ, analysis of CCR7 IHC was performed on color deconvolved (Image J Plugin) DAB-hematoxylin images. Plot intensity measurement on DAB isolated images were performed up to 150 μm from the edge of the scaffold to obtain measurements for CCR7 staining intensity.

### Wound Scoring

Treatment-blinded histomorphometric analysis of trichrome- and H&E-stained tissue sections was used for histological scoring of wounds based on a composite evaluation of five parameters: extent of granulation tissue formation (G), degree of vascularization (V), re-epithelialization (E), collagen deposition (amount and fiber orientation) (C), and inflammation (I) as shown in Table 2. A serial section stained with trichrome stain was scored to estimate the extent of and organization of new connective tissue. Each parameter was rated on a 1-5 scale, ranging from little or no activity to near-complete reorganization of tissue. The overall score comprised of G+V+E+C−(½) was assigned to each wound with a higher score indicating a more favorable wound healing response. Semi-quantitative analysis for the formation of foreign body giant cells (FBGC) was performed on the group of wounds treated with EG7 PTK-UR, NovoSorb, and Integra. The foreign body response was scored by evaluation of hematoxylin and eosin-stained tissue sections. A score of 0 indicated little or no association of FBGC with residual scaffold fragments, and a score of 5 represented intense association of both mononuclear cells and FBGC with persistent scaffold material. In the current series, the maximum FBGC reaction score was 3. In this experiment, an additional FBGC score between 1-5 was assigned to each wound and subtracted from the total wound healing score to account for foreign body response to the implanted materials.

### Gene Expression Analysis of Granulation Tissue

Wound samples were excised at predetermined time points and stored in RNAlater Stabilizing Solution (Invitrogen) at −20° C. Total RNA was extracted from tissue using QIAzol Lysis Reagent (Qiagen). Briefly, tissue samples were minced and homogenized in QIAzol using a Tissue Lyser (Qiagen). Tissue homogenate was phase separated using chloroform, and the aqueous RNA layer was collected and processed using a RNeasy Mini kit (Qiagen) to eliminate phenolic impurities carried over from QIAzol extraction. During the column-based cleanup process, on-column DNase treatment was performed to eliminate genomic DNA from the samples. RNA samples were reverse transcribed to cDNA and quantified using a custom porcine Qiagen PCR Array Kit. Genes involved in various wound healing phases including inflammatory cytokines and interleukins, transcription factors, cell surface markers, ECM components, growth factors and remodeling enzymes were included in the panel.

### Mouse Subcutaneous Scaffold Implantation

All surgical procedures were approved by the Vanderbilt University Institutional Animal Care and Use Committee. The ventral region of C57BL/6 mice was shaved and prepped with betadine and ethanol, followed by the creation of small incisions, one on either side of the midline. The incisions allowed for the dissection of subcutaneous pockets for scaffold implantation. EG2 PTK-UR and EG7 PTK-UR scaffolds were trimmed to 6×1 mm discs, UV sterilized, and implanted in the subcutaneous pockets that were then sutured close to secure the scaffolds in place. Each mouse received 2 scaffolds of the same formulation. Mice were similarly implanted with 900t PE-UR and EG7 PTK-UR scaffolds to assess relative ROS expression associated with each scaffold chemistry in vivo.

Assessment of H₂O₂ Levels in Mice Using Amplex Red

Mice were euthanized 4 days post-implant to retrieve scaffolds from the subcutaneous space. The explanted scaffolds were weighed, minced, and incubated in 0.5 mL of the Amplex Red reaction mixture (prepared according to kit instructions) for 1 hour at room temperature. Following incubation, the fluorescence intensity of the sample supernatants were measured, and the average fluorescence measures from each sample was normalized to the scaffold mass.

### Mouse Scaffold Retrieval and Cell Isolation

Mice were euthanized 7, 14, and 21 days post-surgery to retrieve scaffolds from the subcutaneous sites. Cells from two scaffolds of the same formulation, implanted in the same mice were pooled for analysis. Scaffolds from each mouse were finely minced in 1 mL PBS and the resultant tissue slurry was digested in a solution of Collagenase D (2 mg/mL, Sigma Aldrich), DNase1 (0.1 mg/mL, Sigma Aldrich), and Liberase TL (0.7 mg/mL, Sigma Aldrich) for 30 minutes at 37° C. on an orbital shaker. The digested cell solution was filtered through a 100 μm cell strainer to removed undigested tissue, treated with ACK lysis buffer (ThermoFisher Scientific) to remove red blood cells, and washed with cold PBS supplemented with 2 mM EDTA and 1% FBS. The cell suspension was pelleted and resuspended at 2×10\hat{ }⁶ cells/mL. 250 μL of the cell suspension was transferred into polystyrene tubes for antibody staining for each of the two panels.

### Flow Cytometry

The suspended cells from each treatment and time point were blocked with Fc block (Invitrogen) for 5 mins and stained for myeloid cell surface markers using the following antibody panel: DAPI, CD45 BriliantViolet 510, F4/80 FITC, CD206 PE, CD11c PerCP-Cy5.5, CD86 PE-Cy7, CD301b eFluor660, and CD11b APC-Cy7 (FIG. 30B) for 30 minutes at 4° C. Cells were then washed with 1% FBS in PBS before analysis. All cell populations were gated on live immune cells (DAPI/CD45) prior to phenotype specific gating.

For the lymphoid analysis, cells were stained with Live/DEAD Fixable dye (Invitrogen) followed by a solution of surface markers for 30 minutes at 4° C. including CD45 BriliantViolet 510, CD8a FITC, TCRγδ PE-Cy7, CD4 APC, and TCRβ APC-Cy7 (FIG. 30B). After staining for surface markers, cells were fixed with True Nuclear Transcription Factor Staining Kit (Biolegend) for 60 minutes at room temperature and stained for FOXP3 PE and GATA3 PerCP-Cy5.5 intracellular markers for an additional 30 minutes. Cells were washed with 1% FBS in PBS before being analyzed. All cell populations were gated on live immune cells (Viability dye/CD45) prior to phenotypic specific gating.

Gates (FIG. 31A) were set using fluorescence minus one (FMO) and positive controls. Leukocytes were defined as live CD45⁺cells; monocytes and neutrophils were defined as CD45⁺CD11b⁺F4/80⁻population. The CD45⁺CD11b⁺F4/80⁺population (pan macrophage population) was further analyzed for the presence of CD206 surface marker and the CD45⁺CD11b⁺F4/80⁺CD206⁺population was isolated. Presence of CD301b surface marker within the CD45⁺CD11b⁺F4/80⁺CD206+ macrophage population was quantified and presented as mean fluorescence intensity (MFI) adapted from Shook et al. (67). Within the CD45⁺CD11b⁺F4/80+ macrophage population, we further analyzed the expression of CD11c surface markers and isolated two population, CD45⁺CD11b⁺CD11c⁺F4/80+(CD11c⁺macrophage) and CD45⁺CD11b⁺CD11c⁻F4/80+(CD11c⁻macrophage population); dendritic (DCs) cells were characterized as CD45⁺CD11c⁺F4/80−. Lymphocytes were separated into TCRβ⁺TCRγδ⁻ and TCRβ⁻TCRγδ⁺to define two lymphoid populations. TCRβ⁺TCRγδ⁻ were further divided into CD8a⁺CD4⁻ and CD8a⁻CD4⁺populations to identify cytotoxic T lymphocytes (CTLs) and helper T (T_{H}) cells respectively. The CD8a⁻CD4⁺T helper cell population was used to identify FoxP3+ and GATA3⁺regulatory T cells and type 2 helper T cells (T_{H}2). All analyses were performed in FlowJo Flowcytometry Analysis Software (Treetar)

### Statistical Analysis

All analyses of qRT PCR data were performed using dCt values calculated from the geometric means of three housekeeping genes, B2M, HPRT1, and RPL13A. PTK treatment fold change (FC) and log₂(FC) were calculated using 2\hat{ }^{-(dCt)} relative to 900t PE for each time point and presented as geometric mean and arithmetic mean respectively. Heatmaps are presented as row-normalized dCt values or log₂(FC) as indicated on each plot. For day 31 gene expression comparison, log₂(FC) of EG7 PTK-UR is given relative to NovoSorb. One-way analyses of variance (ANOVA) followed by Tukey's pairwise comparison was performed using GraphPad Prism to define statistical differences between treatments at a given time point. Kinetic studies were analyzed using two-way analysis of variance (ANOVA) between groups. Welch's t-test was used to compare differences between two groups a given time point. Linear regression was used to analyze correlations between EG content and functional readouts including contact angle in vitro, tissue infiltration in vivo, and blood vessel density in vivo.

All patents, patent applications, published applications and publications, GenBank sequences, databases, websites, and other published materials referred to throughout this specification are, unless noted otherwise, incorporated by reference to the same extent as if each was specifically and individually indicated to be incorporated by reference, including the references set forth in the following list:
1. C. K. Sen, Human Wounds and Its Burden: An Updated Compendium of Estimates. Adv Wound Care (New Rochelle) 8, 39-48 (2019).
2. S. R. Nussbaum, M. J. Carter, C. E. Fife, J. DaVanzo, R. Haught, M. Nusgart, D. Cartwright, An Economic Evaluation of the Impact, Cost, and Medicare Policy Implications of Chronic Nonhealing Wounds. Value in Health 21, 27-32 (2018).
3. R. G. Frykberg, J. Banks, Challenges in the Treatment of Chronic Wounds. Adv Wound Care (New Rochelle) 4, 560-582 (2015).
4. S. Matoori, A. Veves, D. J. Mooney, Advanced bandages for diabetic wound healing. Sci Transl Med 13, (2021).
5. N. A. Richmond, A. D. Maderal, A. C. Vivas, Evidence-based management of common chronic lower extremity ulcers. Dermatol Ther 26, 187-196 (2013).
6. D. C. Jupiter, J. C. Thorud, C. J. Buckley, N. Shibuya, The impact of foot ulceration and amputation on mortality in diabetic patients. I: From ulceration to death, a systematic review. Int Wound J 13, 892-903 (2016).
7. T. N. Demidova-Rice, M. R. Hamblin, I. M. Herman, Acute and impaired wound healing: pathophysiology and current methods for drug delivery, part 1: normal and chronic wounds: biology, causes, and approaches to care. Adv Skin Wound Care 25, 304-314 (2012).
8. S. M. Peirce, T. C. Skalak, G. T. Rodeheaver, Ischemia-reperfusion injury in chronic pressure ulcer formation: a skin model in the rat. Wound Repair Regen 8, 68-76 (2000).
9. M. Mittal, M. R. Siddiqui, K. Tran, S. P. Reddy, A. B. Malik, Reactive oxygen species in inflammation and tissue injury. Antioxid Redox Signal 20, 1126-1167 (2014).
10. K. Kirketerp-Moller, P. O. Jensen, M. Fazli, K. G. Madsen, J. Pedersen, C. Moser, T. Tolker-Nielsen, N. Hoiby, M. Givskov, T. Bjarnsholt, Distribution, organization, and ecology of bacteria in chronic wounds. J Clin Microbiol 46, 2717-2722 (2008).
11. R. E. Jones, D. S. Foster, M. T. Longaker, Management of Chronic Wounds-2018. JAMA 320, 1481-1482 (2018).
12. M. Sheikholeslam, M. E. E. Wright, M. G. Jeschke, S. Amini-Nik, Biomaterials for Skin Substitutes. Adv Healthc Mater 7, (2018).
13. I. V. Yannas, J. F. Burke, D. P. Orgill, E. M. Skrabut, Wound Tissue Can Utilize a Polymeric Template to Synthesize a Functional Extension of Skin. Science 215, 174-176 (1982).
14. V. R. Driver, L. A. Lavery, A. M. Reyzelman, T. G. Dutra, C. R. Dove, S. V. Kotsis, H. M. Kim, K. C. Chung, A clinical trial of Integra Template for diabetic foot ulcer treatment. Wound Repair Regen 23, 891-900 (2015).
15. E. N. Mostow, G. D. Haraway, M. Dalsing, J. P. Hodde, D. King, O. V. U. S. Group, Effectiveness of an extracellular matrix graft (OASIS Wound Matrix) in the treatment of chronic leg ulcers: a randomized clinical trial. J Vasc Surg 41, 837-843 (2005).
16. E. Davison-Kotler, V. Sharma, N. V. Kang, E. Garcia-Gareta, A Universal Classification System of Skin Substitutes Inspired by Factorial Design. Tissue Eng Part B Rev 24, 279-288 (2018).
17. S. Shakir, C. A. t. Messa, R. B. Broach, I. A. Rhemtulla, B. Chatman, A. D′Angelantonio, L. S. Levin, S. J. Kovach, 3rd, J. M. Serletti, J. P. Fischer, Indications and Limitations of Bilayer Wound Matrix-Based Lower Extremity Reconstruction: A Multidisciplinary Case-Control Study of 191 Wounds. Plast Reconstr Surg 145, 813-822 (2020).
18. X. Li, B. Cho, R. Martin, M. Seu, C. Zhang, Z. Zhou, J. S. Choi, X. Jiang, L. Chen, G. Walia, J. Yan, M. Callanan, H. Liu, K. Colbert, J. Morrissette-McAlmon, W. Grayson, S. Reddy, J. M. Sacks, H. Q. Mao, Nanofiber-hydrogel composite-mediated angiogenesis for soft tissue reconstruction. Sci Transl Med 11, (2019).
19. A. Y. Clark, K. E. Martin, J. R. Garcia, C. T. Johnson, H. S. Theriault, W. M. Han, D. W. Zhou, E. A. Botchwey, A. J. Garcia, Integrin-specific hydrogels modulate transplanted human bone marrow-derived mesenchymal stem cell survival, engraftment, and reparative activities. Nature Communications 11, (2020).
20. S. Zhu, S. Li, H. Escuin-Ordinas, R. Dimatteo, W. Xi, A. Ribas, T. Segura, Accelerated wound healing by injectable star poly(ethylene glycol)-b-poly(propylene sulfide) scaffolds loaded with poorly water-soluble drugs. J Control Release 282, 156-165 (2018).
21. M. Mir, M. N. Ali, A. Barakullah, A. Gulzar, M. Arshad, S. Fatima, M. Asad, Synthetic polymeric biomaterials for wound healing: a review. Prog Biomater 7, 1-21 (2018).
22. A. Li, B. L. Dearman, K. E. Crompton, T. G. Moore, J. E. Greenwood, Evaluation of a novel biodegradable polymer for the generation of a dermal matrix. J Burn Care Res 30, 717-728 (2009).
23. M. R. MacEwan, S. MacEwan, A. P. Wright, T. R. Kovacs, J. Batts, L. Zhang, Comparison of a Fully Synthetic Electrospun Matrix to a Bi-Layered Xenograft in Healing Full Thickness Cutaneous Wounds in a Porcine Model. Cureus 9, e1614 (2017).
24. K. Fogh, J. Nielsen, Clinical utility of foam dressings in wound management: a review. Chronic Wound Care Management and Research, 31 (2015).
25. L. Li, X. Liu, Y. Niu, J. Ye, S. Huang, C. Liu, K. Xu, Synthesis and wound healing of alternating block polyurethanes based on poly(lactic acid) (PLA) and poly(ethylene glycol) (PEG). J Biomed Mater Res B Appl Biomater 105, 1200-1209 (2017).
26. T. Laube, J. Weisser, S. Berger, S. Borner, S. Bischoff, H. Schubert, M. Gajda, R. Brauer, M. Schnabelrauch, In situ foamable, degradable polyurethane as biomaterial for soft tissue repair. Mater Sci Eng C Mater Biol Appl 78, 163-174 (2017).
27. I. C. Bonzani, R. Adhikari, S. Houshyar, R. Mayadunne, P. Gunatillake, M. M. Stevens, Synthesis of two-component injectable polyurethanes for bone tissue engineering. Biomaterials 28, 423-433 (2007).
28. E. J. Adolph, A. C. Pollins, N. L. Cardwell, J. M. Davidson, S. A. Guelcher, L. B. Nanney, Biodegradable lysine-derived polyurethane scaffolds promote healing in a porcine full-thickness excisional wound model. J Biomater Sci Polym Ed 25, 1973-1985 (2014).
29. C. E. Nelson, A. J. Kim, E. J. Adolph, M. K. Gupta, F. Yu, K. M. Hocking, J. M. Davidson, S. A. Guelcher, C. L. Duvall, Tunable delivery of siRNA from a biodegradable scaffold to promote angiogenesis in vivo. Adv Mater 26, 607-614, 506 (2014).
30. A. E. Hafeman, B. Li, T. Yoshii, K. Zienkiewicz, J. M. Davidson, S. A. Guelcher, Injectable biodegradable polyurethane scaffolds with release of platelet-derived growth factor for tissue repair and regeneration. Pharm Res 25, 2387-2399 (2008).
31. L. N. Woodard, M. A. Grunlan, Hydrolytic Degradation and Erosion of Polyester Biomaterials. ACS Macro Lett 7, 976-982 (2018).
32. K. Ceonzo, A. Gaynor, L. Shaffer, K. Kojima, C. A. Vacanti, G. L. Stahl, Polyglycolic acid-induced inflammation: role of hydrolysis and resulting complement activation. Tissue Eng 12, 301-308 (2006).
33. F. El-Mohtadi, R. d'Arcy, N. Tirelli, Oxidation-Responsive Materials: Biological Rationale, State of the Art, Multiple Responsiveness, and Open Issues. Macromol Rapid Commun 40, e1800699 (2019).
34. J. R. Martin, M. K. Gupta, J. M. Page, F. Yu, J. M. Davidson, S. A. Guelcher, C. L. Duvall, A porous tissue engineering scaffold selectively degraded by cell-generated reactive oxygen species. Biomaterials 35, 3766-3776 (2014).
35. P. Patil, J. R. Martin, S. M. Sarett, A. C. Pollins, N. L. Cardwell, J. M. Davidson, S. A. Guelcher, L. B. Nanney, C. L. Duvall, Porcine Ischemic Wound-Healing Model for Preclinical Testing of Degradable Biomaterials. Tissue Eng Part C Methods 23, 754-762 (2017).
36. J. R. Martin, P. Patil, F. Yu, M. K. Gupta, C. L. Duvall, Enhanced stem cell retention and antioxidative protection with injectable, ROS-degradable PEG hydrogels. Biomaterials 263, 120377 (2020).
37. A. E. Hafeman, K. J. Zienkiewicz, A. L. Zachman, H. J. Sung, L. B. Nanney, J. M. Davidson, S. A. Guelcher, Characterization of the degradation mechanisms of lysine-derived aliphatic poly(ester urethane) scaffolds. Biomaterials 32, 419-429 (2011).
38. L. Zhang, Z. Cao, T. Bai, L. Carr, J. R. Ella-Menye, C. Irvin, B. D. Ratner, S. Jiang, Zwitterionic hydrogels implanted in mice resist the foreign-body reaction. Nat Biotechnol 31, 553-556 (2013).
39. D. S. Wilson, G. Dalmasso, L. Wang, S. V. Sitaraman, D. Merlin, N. Murthy, Orally delivered thioketal nanoparticles loaded with TNF-alpha-siRNA target inflammation and inhibit gene expression in the intestines. Nat Mater 9, 923-928 (2010).
40. J. R. Martin, C. E. Nelson, M. K. Gupta, F. Yu, S. M. Sarett, K. M. Hocking, A. C. Pollins, L. B. Nanney, J. M. Davidson, S. A. Guelcher, C. L. Duvall, Local Delivery of PHD2 siRNA from ROS-Degradable Scaffolds to Promote Diabetic Wound Healing. Adv Healthc Mater 5, 2751-2757 (2016).
41. M. S. Shim, Y. Xia, R oxygen species (ROS)-responsive polymer for safe, efficient, and targeted gene delivery in cancer cells. Angew Chem Int Ed Engl 52, 6926-6929 (2013).
42. C. F. Guimaraes, L. Gasperini, A. P. Marques, R. L. Reis, The stiffness of living tissues and its implications for tissue engineering. Nature Reviews Materials 5, 351-370 (2020).
43. L. Penuela, C. Negro, M. Massa, E. Repaci, E. Cozzani, A. Parodi, S. Scaglione, R. Quarto, R. Raiteri, Atomic force microscopy for biomechanical and structural analysis of human dermis: A complementary tool for medical diagnosis and therapy monitoring. Exp Dermatol 27, 150-155 (2018).
44. B. Liu, S. Thayumanavan, Mechanistic Investigation on Oxidative Degradation of ROS-Responsive Thioacetal/Thioketal Moieties and Their Implications. Cell Reports Physical Science 1, (2020).
45. E. M. Sussman, M. C. Halpin, J. Muster, R. T. Moon, B. D. Ratner, Porous implants modulate healing and induce shifts in local macrophage polarization in the foreign body reaction. Ann Biomed Eng 42, 1508-1516 (2014).
46. J. M. Anderson, A. Rodriguez, D. T. Chang, Foreign body reaction to biomaterials. Semin Immunol 20, 86-100 (2008).
47. W. K. Ward, A Review of the Foreign-body Response to Subcutaneously-implanted Devices: The Role of Macrophages and Cytokines in Biofouling and Fibrosis. Journal of Diabetes Science and Technology 2, (2008).
48. S. Jhunjhunwala, Neutrophils at the Biological-Material Interface. ACS Biomater Sci Eng 4, 1128-1136 (2018).
49. G. S. Selders, A. E. Fetz, M. Z. Radic, G. L. Bowlin, An overview of the role of neutrophils in innate immunity, inflammation and host-biomaterial integration. Regen Biomater 4, 55-68 (2017).
50. A. Valavanidis, T. Vlachogianni, C. Fiotakis, 8-hydroxy-2′-deoxyguanosine (8-OHdG): A critical biomarker of oxidative stress and carcinogenesis. J Environ Sci Health C Environ Carcinog Ecotoxicol Rev 27, 120-139 (2009).
51. B. N. Brown, B. D. Ratner, S. B. Goodman, S. Amar, S. F. Badylak, Macrophage polarization: an opportunity for improved outcomes in biomaterials and regenerative medicine. Biomaterials 33, 3792-3802 (2012).
52. S. F. Badylak, J. E. Valentin, A. K. Ravindra, G. P. McCabe, A. M. Stewart-Akers, Macrophage phenotype as a determinant of biologic scaffold remodeling. Tissue Eng Part A 14, 1835-1842 (2008).
53. P. Rousselle, F. Braye, G. Dayan, Re-epithelialization of adult skin wounds: Cellular mechanisms and therapeutic strategies. Adv Drug Deliv Rev, (2018).
54. I. Pastar, O. Stojadinovic, N. C. Yin, H. Ramirez, A. G. Nusbaum, A. Sawaya, S. B. Patel, L. Khalid, R. R. Isseroff, M. Tomic-Canic, Epithelialization in Wound Healing: A Comprehensive Review. Adv Wound Care (New Rochelle) 3, 445-464 (2014).
55. M. L. Usui, J. N. Mansbridge, W. G. Carter, M. Fujita, J. E. Olerud, Keratinocyte migration, proliferation, and differentiation in chronic ulcers from patients with diabetes and normal wounds. J Histochem Cytochem 56, 687-696 (2008).
56. K. Sadtler, K. Estrellas, B. W. Allen, M. T. Wolf, H. Fan, A. J. Tam, C. H. Patel, B. S. Luber, H. Wang, K. R. Wagner, J. D. Powell, F. Housseau, D. M. Pardoll, J. H. Elisseeff, Developing a pro-regenerative biomaterial scaffold microenvironment requires T helper 2 cells. Science 352, 366-370 (2016).
57. N. Strbo, N. Yin, O. Stojadinovic, Innate and Adaptive Immune Responses in Wound Epithelialization. Adv Wound Care (New Rochelle) 3, 492-501 (2014).
58. A. S. MacLeod, J. N. Mansbridge, The Innate Immune System in Acute and Chronic Wounds. Adv Wound Care (New Rochelle) 5, 65-78 (2016).
59. L. Chung, D. R. Maestas, Jr., A. Lebid, A. Mageau, G. D. Rosson, X. Wu, M. T. Wolf, A. J. Tam, I. Vanderzee, X. Wang, J. I. Andorko, H. Zhang, R. Narain, K. Sadtler, H. Fan, D. Cihakova, C. J. Le Saux, F. Housseau, D. M. Pardoll, J. H. Elisseeff, Interleukin 17 and senescent cells regulate the foreign body response to synthetic material implants in mice and humans. Sci Transl Med 12, (2020).
60. O. J. Harrison, J. L. Linehan, H. Y. Shih, N. Bouladoux, S. J. Han, M. Smelkinson, S. K. Sen, A. L. Byrd, M. Enamorado, C. Yao, S. Tamoutounour, F. Van Laethem, C. Hurabielle, N. Collins, A. Paun, R. Salcedo, J. J. O'Shea, Y. Belkaid, Commensal-specific T cell plasticity promotes rapid tissue adaptation to injury. Science 363, (2019).
61. J. C. Doloff, O. Veiseh, A. J. Vegas, H. H. Tam, S. Farah, M. Ma, J. Li, A. Bader, A. Chiu, A. Sadraei, S. Aresta-Dasilva, M. Griffin, S. Jhunjhunwala, M. Webber, S. Siebert, K. Tang, M. Chen, E. Langan, N. Dholokia, R. Thakrar, M. Qi, J. Oberholzer, D. L. Greiner, R. Langer, D. G. Anderson, Colony stimulating factor-1 receptor is a central component of the foreign body response to biomaterial implants in rodents and non-human primates. Nat Mater 16, 671-680 (2017).
62. J. R. Martin, (2017).
63. R. M. Visalakshan, M. N. MacGregor, S. Sasidharan, A. Ghazaryan, A. M. Mierczynska-Vasilev, S. Morsbach, V. Mailander, K. Landfester, J. D. Hayball, K. Vasilev, Biomaterial Surface Hydrophobicity-Mediated Serum Protein Adsorption and Immune Responses. ACS Appl Mater Interfaces 11, 27615-27623 (2019).
64. J. Y. Hsieh, T. D. Smith, V. S. Meli, T. N. Tran, E. L. Botvinick, W. F. Liu, Differential regulation of macrophage inflammatory activation by fibrin and fibrinogen. Acta Biomater 47, 14-24 (2017).
65. D. Gay, O. Kwon, Z. Zhang, M. Spata, M. V. Plikus, P. D. Holler, M. Ito, Z. Yang, E. Treffeisen, C. D. Kim, A. Nace, X. Zhang, S. Baratono, F. Wang, D. M. Ornitz, S. E. Millar, G. Cotsarelis, Fgf9 from dermal gammadelta T cells induces hair follicle neogenesis after wounding. Nat Med 19, 916-923 (2013).
66. J. Jameson, K. Ugarte, N. Chen, P. Yachi, E. Fuchs, R. Boismenu, W. L. Havran, A role for skin gammadelta T cells in wound repair. Science 296, 747-749 (2002).
67. B. Shook, E. Xiao, Y. Kumamoto, A. Iwasaki, V. Horsley, CD301b+ Macrophages Are Essential for Effective Skin Wound Healing. J Invest Dermatol 136, 1885-1891 (2016).
68. S. D. Sommerfeld, C. Cherry, R. M. Schwab, L. Chung, D. R. Maestas, P. Laffont, J. E. Stein, A. Tam, S. Ganguly, F. Housseau, J. M. Taube, D. M. Pardoll, P. Cahan, J. H. Elisseeff, Interleukin-36 gamma-producing macrophages drive IL-17-mediated fibrosis. Sci Immunol 4, (2019).
69. B. A. Shook, R. R. Wasko, G. C. Rivera-Gonzalez, E. Salazar-Gatzimas, F. Lopez-Giraldez, B. C. Dash, A. R. Munoz-Rojas, K. D. Aultman, R. K. Zwick, V. Lei, J. L. Arbiser, K. Miller-Jensen, D. A. Clark, H. C. Hsia, V. Horsley, Myofibroblast proliferation and heterogeneity are supported by macrophages during skin repair. Science 362, (2018).
70. L. Koivisto, J. Heino, L. Hakkinen, H. Larjava, Integrins in Wound Healing. Adv Wound Care (New Rochelle) 3, 762-783 (2014).
71. K. Switala-Jelen, K. Dabrowska, A. Opolski, L. Lipinska, M. Nowaczyk, A. Gorski, The biological functions of beta3 integrins. Folia Biol (Praha) 50, 143-152 (2004).
72. R. J. Akhurst, A. Hata, Targeting the TGFbeta signalling pathway in disease. Nat Rev Drug Discov 11, 790-811 (2012).
73. H. Nagase, R. Visse, G. Murphy, Structure and function of matrix metalloproteinases and TIMPs. Cardiovasc Res 69, 562-573 (2006).
74. M. P. Caley, V. L. Martins, E. A. O'Toole, Metalloproteinases and Wound Healing. Adv Wound Care (New Rochelle) 4, 225-234 (2015).
75. M. Vaalamo, L. Mattila, N. Johansson, A. L. Kariniemi, M. L. Karjalainen-Lindsberg, V. M. Kahari, U. Saarialho-Kere, Distinct populations of stromal cells express collagenase-3 (MMP-13) and collagenase-1 (MMP-1) in chronic ulcers but not in normally healing wounds. J Invest Dermatol 109, 96-101 (1997).
76. M. Vaalamo, T. Leivo, U. Saarialho-Kere, Differential expression of tissue inhibitors of metalloproteinases (TIMP-1, -2, -3, and -4) in normal and aberrant wound healing. Hum Pathol 30, 795-802 (1999).
77. S. Barrientos, O. Stojadinovic, M. S. Golinko, H. Brem, M. Tomic-Canic, Growth factors and cytokines in wound healing. Wound Repair Regen 16, 585-601 (2008).
78. A. B. Molofsky, A. K. Savage, R. M. Locksley, Interleukin-33 in Tissue Homeostasis, Injury, and Inflammation. Immunity 42, 1005-1019 (2015).
79. T. Li, Z. Zhang, J. G. Bartolacci, G. K. Dwyer, Q. Liu, L. R. Mathews, M. Velayutham, A. S. Roessing, Y. C. Lee, H. Dai, S. Shiva, M. H. Oberbarnscheidt, J. L. Dziki, S. J. Mullet, S. G. Wendell, J. D. Wilkinson, S. A. Webber, M. Wood-Trageser, S. C. Watkins, A. J. Demetris, G. S. Hussey, S. F. Badylak, H. R. Turnquist, Graft IL-33 regulates infiltrating macrophages to protect against chronic rejection. J Clin Invest 130, 5397-5412 (2020).
80. L. Bosurgi, Y. G. Cao, M. Cabeza-Cabrerizo, A. Tucci, L. D. Hughes, Y. Kong, J. S. Weinstein, P. Licona-Limon, E. T. Schmid, F. Pelorosso, N. Gagliani, J. E. Craft, R. A. Flavell, S. Ghosh, C. V. Rothlin, Macrophage function in tissue repair and remodeling requires IL-4 or IL-13 with apoptotic cells. Science 356, 1072-1076 (2017).
81. R. L. Gieseck, 3rd, M. S. Wilson, T. A. Wynn, Type 2 immunity in tissue repair and fibrosis. Nat Rev Immunol 18, 62-76 (2018).
82. D. R. Griffin, M. M. Archang, C. H. Kuan, W. M. Weaver, J. S. Weinstein, A. C. Feng, A. Ruccia, E. Sideris, V. Ragkousis, J. Koh, M. V. Plikus, D. Di Carlo, T. Segura, P. O. Scumpia, Activating an adaptive immune response from a hydrogel scaffold imparts regenerative wound healing. Nat Mater, (2020).
83. M. L. Sabolinski, G. Gibbons, Comparative effectiveness of a bilayered living cellular construct and an acellular fetal bovine collagen dressing in the treatment of venous leg ulcers. J Comp Eff Res 7, 797-805 (2018).
84. L. Zaulyanov, R. S. Kirsner, A review of a bi-layered living cell treatment (Apligraf) in the treatment of venous leg ulcers and diabetic foot ulcers. Clin Interv Aging 2, 93-98 (2007).
85. B. Samsell, J. McLean, S. Cazzell, K. Dorsch, P. M. Moyer, M. Moore, Health economics for treatment of diabetic foot ulcers: a cost-effectiveness analysis of eight skin substitutes. J Wound Care 28, S14-S26 (2019).
86. H. Zhang, L. Zhou, W. Zhang, Control of scaffold degradation in tissue engineering: a review. Tissue Eng Part B Rev 20, 492-502 (2014).
87. J. Alijotas-Reig, M. T. Fernindez-Figueras, L. Puig, Late-Onset Inflammatory Adverse Reactions Related to Soft Tissue Filler Injections. Clinical Reviews in Allergy & Immunology 45, 97-108 (2013).
88. R. Moseley, J. R. Hilton, R. J. Waddington, K. G. Harding, P. Stephens, D. W. Thomas, Comparison of oxidative stress biomarker profiles between acute and chronic wound environments. Wound Repair Regen 12, 419-429 (2004).
89. T. J. James, M. A. Hughes, G. W. Cherry, R. P. Taylor, Evidence of oxidative stress in chronic venous ulcers. Wound Repair Regen 11, 172-176 (2003).
90. M. Schafer, S. Werner, Oxidative stress in normal and impaired wound repair. Pharmacol Res 58, 165-171 (2008).
91. S. J. Forrester, D. S. Kikuchi, M. S. Hernandes, Q. Xu, K. K. Griendling, Reactive Oxygen Species in Metabolic and Inflammatory Signaling. Circ Res 122, 877-902 (2018).
92. C. Dunnill, T. Patton, J. Brennan, J. Barrett, M. Dryden, J. Cooke, D. Leaper, N. T. Georgopoulos, Reactive oxygen species (ROS) and wound healing: the functional role of ROS and emerging ROS-modulating technologies for augmentation of the healing process. Int Wound J 14, 89-96 (2017).
93. A. K. Blakney, M. D. Swartzlander, S. J. Bryant, The effects of substrate stiffness on the in vitro activation of macrophages and in vivo host response to poly(ethylene glycol)-based hydrogels. J Biomed Mater Res A 100, 1375-1386 (2012).
94. C. M. Walthers, A. K. Nazemi, S. L. Patel, B. M. Wu, J. C. Dunn, The effect of scaffold macroporosity on angiogenesis and cell survival in tissue-engineered smooth muscle. Biomaterials 35, 5129-5137 (2014).
95. C. Krisp, F. Jacobsen, M. J. McKay, M. P. Molloy, L. Steinstraesser, D. A. Wolters, Proteome analysis reveals antiangiogenic environments in chronic wounds of diabetes mellitus type 2 patients. Proteomics 13, 2670-2681 (2013).
96. A. N. Stratman, A. E. Schwindt, K. M. Malotte, G. E. Davis, Endothelial-derived PDGF-BB and HB-EGF coordinately regulate pericyte recruitment during vasculogenic tube assembly and stabilization. Blood 116, 4720-4730 (2010).
97. R. Cao, E. Brakenhielm, R. Pawliuk, D. Wariaro, M. J. Post, E. Wahlberg, P. Leboulch, Y. Cao, Angiogenic synergism, vascular stability and improvement of hind-limb ischemia by a combination of PDGF-BB and FGF-2. Nat Med 9, 604-613 (2003).
98. P. Carmeliet, Angiogenesis in health and disease. Nat Med 9, 653-660 (2003).
99. N. Muraoka, L. Shum, S. Fukumoto, T. Nomura, M. Ohishi, K. Nonaka, Transforming growth factor-β promotes mesenchymal cell proliferation and angiogenesis mediated by the enhancement of cyclin D1, Flk-1, and CD31 gene expression during CL/Fr mouse lip fusion. Birth Defects Research Part A: Clinical and Molecular Teratology 73, 956-965 (2005).
100. L. Lv, Y. Xie, K. Li, T. Hu, X. Lu, Y. Cao, X. Zheng, Unveiling the Mechanism of Surface Hydrophilicity-Modulated Macrophage Polarization. Adv Healthc Mater 7, e1800675 (2018).
101. S. A. Eming, M. Hammerschmidt, T. Krieg, A. Roers, Interrelation of immunity and tissue repair or regeneration. Semin Cell Dev Biol 20, 517-527 (2009).
102. S. Duprd-Crochet, M. Erard, O. Nüβe, ROS production in phagocytes: why, when, and where? Journal of Leukocyte Biolog 94, 657-670 (2013).
103. O. Veiseh, J. C. Doloff, M. Ma, A. J. Vegas, H. H. Tam, A. R. Bader, J. Li, E. Langan, J. Wyckoff, W. S. Loo, S. Jhunjhunwala, A. Chiu, S. Siebert, K. Tang, J. Hollister-Lock, S. Aresta-Dasilva, M. Bochenek, J. Mendoza-Elias, Y. Wang, M. Qi, D. M. Lavin, M. Chen, N. Dholakia, R. Thakrar, I. Lacik, G. C. Weir, J. Oberholzer, D. L. Greiner, R. Langer, D. G. Anderson, Size- and shape-dependent foreign body immune response to materials implanted in rodents and non-human primates. Nat Mater 14, 643-651 (2015).
104. O. Veiseh, A. J. Vegas, Domesticating the foreign body response: Recent advances and applications. Adv Drug Deliv Rev 144, 148-161 (2019).
105. W. G. Brodbeck, J. Patel, G. Voskerician, E. Christenson, M. S. Shive, Y. Nakayama, T. Matsuda, N. P. Ziats, J. M. Anderson, Biomaterial adherent macrophage apoptosis is increased by hydrophilic and anionic substrates in vivo. Proc Natl Acad Sci USA 99, 10287-10292 (2002).
106. B. G. Keselowsky, J. S. Lewis, Dendritic cells in the host response to implanted materials. Semin Immunol 29, 33-40 (2017).
107. J. Park, J. E. Babensee, Differential functional effects of biomaterials on dendritic cell maturation. Acta Biomater 8, 3606-3617 (2012).
108. G. D. Winter, Formation of the scab and the rate of epithelization of superficial wounds in the skin of the young domestic pig. Nature 193, 293-294 (1962).
109. B. L. Dearman, A. Li, J. E. Greenwood, Optimization of a polyurethane dermal matrix and experience with a polymer-based cultured composite skin. J Burn Care Res 35, 437-448 (2014).
110. S. Gordon, Alternative activation of macrophages. Nat Rev Immunol 3, 23-35 (2003).
111. S. J. Van Dyken, J. C. Nussbaum, J. Lee, A. B. Molofsky, H. E. Liang, J. L. Pollack, R. E. Gate, G. E. Haliburton, C. J. Ye, A. Marson, D. J. Erle, R. M. Locksley, A tissue checkpoint regulates type 2 immunity. Nat Immunol 17, 1381-1387 (2016).
112. S. Roy, S. Biswas, S. Khanna, G. Gordillo, V. Bergdall, J. Green, C. B. Marsh, L. J. Gould, C. K. Sen, Characterization of a preclinical model of chronic ischemic wound. Physiol Genomics 37, 211-224 (2009).
113. S. A. Abd-El-Aleem, M. W. Ferguson, I. Appleton, S. Kairsingh, E. B. Jude, K. Jones, C. N. McCollum, G. W. Ireland, Expression of nitric oxide synthase isoforms and arginase in normal human skin and chronic venous leg ulcers. J Pathol 191, 434-442 (2000).
114. M. Shah, D. M. Foreman, M. W. Ferguson, Neutralisation of TGF-beta 1 and TGF-beta 2 or exogenous addition of TGF-beta 3 to cutaneous rat wounds reduces scarring. J Cell Sci 108 (Pt 3), 985-1002 (1995).
115. M. Kalani, K. Brismar, B. Fagrell, J. Ostergren, G. Jorneskog, Transcutaneous oxygen tension and toe blood pressure as predictors for outcome of diabetic foot ulcers. Diabetes Care 22, 147-151 (1999).
116. K. W. Larson, C. L. Austin, S. J. Thompson, Treatment of a Full-Thickness Burn Injury With NovoSorb Biodegradable Temporizing Matrix and RECELL Autologous Skin Cell Suspension: A Case Series. J Burn Care Res 41, 215-219 (2020).
117. Y. I. Shen, H. G. Song, A. Papa, J. Burke, S. W. Volk, S. Gerecht, Acellular Hydrogels for Regenerative Burn Wound Healing: Translation from a Porcine Model. J Invest Dermatol 135, 2519-2529 (2015).
118. M. Puthia, M. Butrym, J. Petrlova, A. C. Stromdahl, M. A. Andersson, S. Kjellstrom, A. Schmidtchen, A dual-action peptide-containing hydrogel targets wound infection and inflammation. Sci Transl Med 12, (2020).
119. J. R. Martin, M. T. Howard, S. Wang, A. G. Berger, P. T. Hammond, Oxidation-Responsive, Tunable Growth Factor Delivery from Polyelectrolyte-Coated Implants. Adv Healthc Mater, e2001941 (2021).
120. H. Yang, J. J. Morris, S. T. Lopina, Polyethylene glycol-polyamidoamine dendritic micelle as solubility enhancer and the effect of the length of polyethylene glycol arms on the solubility of pyrene in water. J Colloid Interface Sci 273, 148-154 (2004).
121. B. Li, J. M. Davidson, S. A. Guelcher, The effect of the local delivery of platelet-derived growth factor from reactive two-component polyurethane scaffolds on the healing in rat skin excisional wounds. Biomaterials 30, 3486-3494 (2009).
122. A. Moghimi, I. Omrani, M. R. Nabid, M. Mahmoodi, Quantification of hydroxyl group in polymers containing trace water by 19F NMR spectroscopy. European Polymer Journal 49, 228-234 (2013).
123. Y. Yao, J. Ding, Z. Wang, H. Zhang, J. Xie, Y. Wang, L. Hong, Z. Mao, J. Gao, C. Gao, ROS-responsive polyurethane fibrous patches loaded with methylprednisolone (MP) for restoring structures and functions of infarcted myocardium in vivo. Biomaterials 232, 119726 (2020).

It will be understood that various details of the presently disclosed subject matter can be changed without departing from the scope of the subject matter disclosed herein. Furthermore, the foregoing description is for the purpose of illustration only, and not for the purpose of limitation.

1. A hydrophilic thioketal polymer comprising a structure according to Formula I: 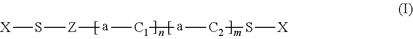
wherein a comprises a thioketal;
wherein C₁ comprises a zwitterionic monomer;
wherein C₂ comprises poly(ethylene glycol) (PEG);
wherein x comprises OH, NH₂, SH, hydroxyalkyl, aminoalkyl, or thioalkyl;
wherein Z comprises —C₁—, —C₂—, or —C₁-a-C₂—;
wherein n is between 0 and 15;
wherein m is between 0 and 15; and
wherein if one of either n or m is 0 the other is at least 1.

2. The polymer of claim 1, wherein the thioketal comprises the following structure:

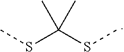

3. The polymer of claim 1, wherein the zwitterionic monomer comprises: 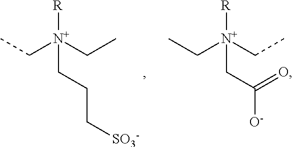
multiples thereof, or combinations thereof.

4. The polymer of claim 1, wherein the PEG comprises 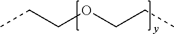
and y is between 0 and 2.

5. The polymer of claim 1, wherein X comprises a hydroxyalkyl.

6. The polymer of claim 5, wherein the hydroxyalkyl comprises a hydroxy(C₁-C₆)alkyl.

7. The polymer of claim 6, wherein the hydroxyalkyl comprises hydroxyethyl.

8. The polymer of claim 1, wherein n is 0.

9. The polymer of claim 1, wherein Z is —C₂—.

10. The polymer of claim 9, wherein the polymer is selected from the group consisting of:

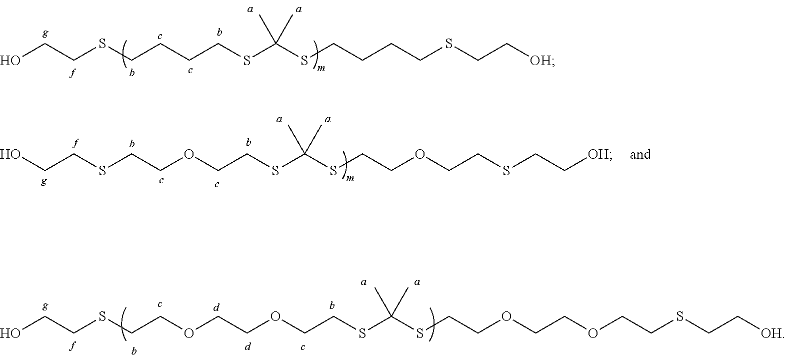

11. The polymer of claim 1, wherein both n and m are greater than 0.

12. The polymer of claim 11, wherein Z is —C₂—.

13. The polymer of claim 12, wherein the polymer comprises:

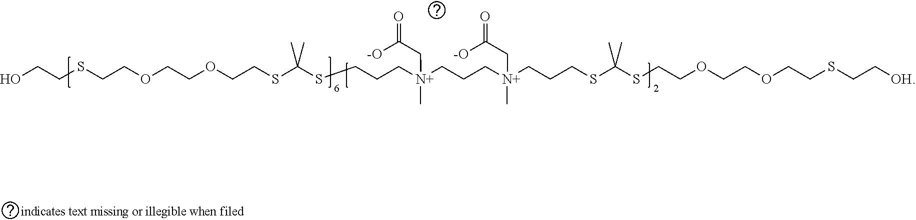

14. The polymer of claim 11, wherein Z is —C₁-a-C₂—.

15. The polymer of claim 14, wherein the polymer comprises:

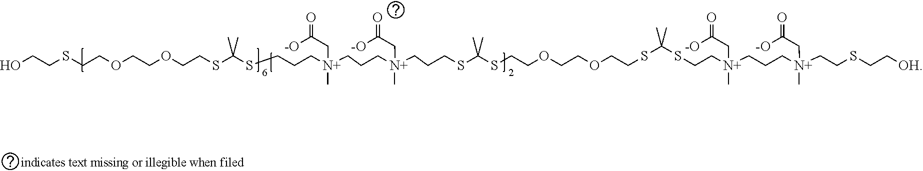

16. A hydrophilic thioketal polymer comprising a structure according to Formula II: 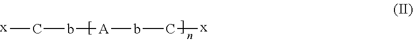
wherein A comprises a thioketal crosslinker;
wherein b comprises carbamate or carbonate;
wherein C comprises poly(ethylene glycol) (PEG);
wherein x comprises OH, NH₂, SH, hydroxyalkyl, aminoalkyl, or thioalkyl; and
wherein n is between 1 and 10.

17. The polymer of claim 16, wherein the PEG comprises 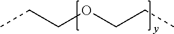
and y is between 3 and 15.

18. The polymer of claim 17, wherein y is at least 7.

19. A hydrophilic thioketal polymer comprising a structure according to Formula III: 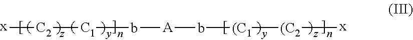
wherein A comprises a thioketal crosslinker;
wherein b comprises a 2° or 3° amide;
wherein C₁ comprises a poly(peptide);
wherein C₂ comprises a poly(peptoid);
wherein x comprises OH, NH₂, SH, hydroxyalkyl, aminoalkyl, or thioalkyl;
wherein y is from about 0 to about 1;
wherein z is from about 1 to about 0; and
wherein n is between 1 and 15.

20. The polymer of claim 19, wherein the polymer comprises: [chemical structure]
